(19) United States
(12) Patent Application Publication
(10) Pub. No.: US 2025/0236673 A1
(43) Pub. Date: Jul. 24, 2025

(54) PROTEIN-BASED CARRIERS FOR SITE-SPECIFIC AMINE CONJUGATION

(71) Applicant: Ablynx N.V., Zwijnaarde (BE)

(72) Inventor: Francis Descamps, Gent (BE)

(73) Assignee: Ablynx N.V., Zwijnaarde (BE)

(21) Appl. No.: 18/989,623

(22) Filed: Dec. 20, 2024

(30) Foreign Application Priority Data
Dec. 22, 2023 (EP) EP 23 219 924.0

(51) Int. Cl. C07K 16/28; A61K 47/68

(52) U.S. Cl. CPC C07K 16/2815; A61K 47/6803; C07K 2317/569; C07K 2317/92; C07K 2317/94

(57) ABSTRACT

The present technology relates to the field of drug delivery and provides molecules comprising or consisting of at least one ISVD suitable for site-specific conjugation, wherein the ISVD comprises at least one, preferably at least two, attachment point(s) or conjugation site(s). In particular, the technology relates to molecule comprising or consisting of at least one ISVD, wherein the at least one ISVD:
comprises at least one (engineered) lysine at at least one solvent accessible position, wherein the solvent accessible position is not located at positions 43, 64, 75 and/or 83 (according to Kabat), preferably wherein the solvent accessible position is not located at positions 43, 64, 75, 83 and/or 97 (according to Kabat), even more preferably wherein the solvent accessible position is not located at positions 19, 43, 64, 75, 83, 97 and/or 105 (according to Kabat); or
comprises only two lysines in its amino acid sequence, wherein the two lysines are located at positions 43, 64, 75 and/or 83 (according to Kabat);
comprises a single lysine, wherein the single lysine is located at position 43, 64, 75 or 83 (according to Kabat); or
does not comprise any lysine in its sequence, preferably does not comprise any primary amine in its sequence.

### REFERENCE TO AN ELECTRONIC SEQUENCE LISTING

The contents of the electronic sequence listing (A084870240US00-SEQ-JRV.xml; Size:

291,718 bytes; and Date of Creation: Dec. 20, 2024) are herein incorporated by reference in its entirety.

### FIELD OF THE TECHNOLOGY

The present technology relates to molecules comprising or consisting of at least one immunoglobulin single variable domain (ISVD) suitable for site-specific conjugation.

The technology further relates to methods for producing such molecules and ISVDs, nucleic acids encoding such molecules or part of such molecules; to host cells comprising such nucleic acids and/or expressing or capable of expressing such molecules or part of such molecules; to compositions, and in particular to pharmaceutical compositions that comprise such molecules, compounds, nucleic acids and/or host cells; and to uses of such molecules, nucleic acids, host cells and/or compositions, in particular for labelling, prophylactic, therapeutic and/or diagnostic purposes.

### TECHNOLOGICAL BACKGROUND

Protein-based therapeutics (referred to as “Biologicals”) are creating new therapeutic strategies which are hard to achieve via the classic, small molecule-based therapeutics. One fast-growing field comprises conjugation-based therapeutics such as antibody-drug conjugates (ADCs). For instance, Fatima S W. and Khare S K. (“Benefits and challenges of antibody drug conjugates as novel form of chemotherapy”, J Control Release, 2022, 341:555-565) review recent clinical advances of each component of ADCs (antibody/linker/payload) and how the individual component influences the activity of ADCs. This is also shown, e.g., in Rader, C., “Chemically programmed antibodies”, Trends in Biotechnology, 2014, 32 (4). Antibody-drug conjugates typically rely on the high specificity and affinity of the antibodies, their extended circulatory half-life as well as other effector functions (e.g., complement-dependent cytotoxicity (CDC), antibody-dependent cellular cytotoxicity (ADCC) and antibody-dependent cellular phagocytosis (ADCP). Hence, antibody-drug conjugates and related molecules, such as chemically programmed antibodies, are chosen because of at least one of these functionalities (their targeting properties, half-life extension properties and/or other effector functions, as described above).

Currently, the manufacture of biologicals mainly relies on recombinant production and is hence restricted to polypeptide production which can combine naturally into one functional unit (i.e., IgGs). Typical conjugation strategies are based on site specific cysteine or stochastic lysine conjugations (see, e.g., Zhou et al., 2021, Pharmaceuticals, 14:672; Sadiki et al., 2020, Antibody Therapeutics, 3:271). The rise of non-canonical or novel Amino Acids (nAAs) further add options for conjugation methodology (see, e.g., Malyshev and Romesberg, 2015, Angew Chem Int Ed Engl., 54 (41); Zhang et al., 2017, PNAS, 114 1317). However, the production of proteins comprising non-natural amino acids may be a technically challenging exercise, in particular if the protein comprises more than one non-natural amino acids.

Site-specific conjugation of ADCs refers to the process of attaching drug molecules to specific sites on antibody molecules. This process ensures that the drug attaches to the designated location on the antibody, thereby minimizing the chance of random or nonspecific binding. By conjugating a drug to a specific site on the antibody, site-specific conjugation helps maintain the binding affinity and specificity of the antibody for its target antigen, while also optimizing the pharmacokinetics and therapeutic efficacy of the drug. It enables precise control of the drug-to-antibody ratio (DAR), ensuring consistent and well-defined drug loading on each antibody molecule.

Stochastic conjugation generates heterogeneous products with varied numbers of drugs coupled across several possible sites, creating significant challenges for process consistency and product characterization. It is also limited in understanding the relationships between the site/extent of drug loading and ADC attributes such as efficacy, safety, pharmacokinetics, and immunogenicity. The non-specific conjugation would also result in more off-target side-effects, leading to relatively low maximum tolerated dose. Several site-specific ADC technologies have been developed. For instance, site-specific ADC have been generated by using native or engineered amino acids, including cysteines (Biomedicines 2017, 5, 64). However, this has the drawback that alkylation by maleimide is reversible (i.e., deconjugation), which has been observed in vivo (Wei C. et al., “Where did the linker-payload go? a quantitative investigation on the destination of the released linker-payload from an antibody-drug conjugate with a maleimide linker in plasma”, Anal Chem., 2016, 88 (9): 4979-86).

There is thus a need of further therapeutic strategies which allow for site-specific conjugation. In particular, there is a need for technically straightforward, simple and efficient site-specific conjugation technologies.

### SUMMARY OF THE TECHNOLOGY

The current technology aims at providing further tools for site-specific conjugation of different cargos on immunoglobulin single variable domains (ISVDs). In particular, the present technology provides molecules comprising or consisting of at least one ISVD that comprises at least one conjugation site or attachment point, which is a primary amine, e.g., present in the side chain of at least one lysine present on the ISVD or at its N-terminus and suitable for site-specific conjugation. The technology further provides methods to produce the ISVDs of the present technology and uses thereof.

### DETAILED DESCRIPTION OF THE TECHNOLOGY

### Definitions

Unless indicated or defined otherwise, all terms used have their usual meaning in the art, which will be clear to the skilled person. Reference is, for example, made to the standard handbooks, such as Sambrook et al., 1989 (Molecular Cloning: A Laboratory Manual, 2nd Ed., Vols. 1-3, Cold Spring Harbor Laboratory Press), Ausubel et al., 1987 (Current protocols in molecular biology, Green Publishing and Wiley Interscience, New York), Lewin 1985 (Genes II, John Wiley & Sons, New York, N.Y.), Old et al., 1981 (Principles of Gene Manipulation: An Introduction to Genetic Engineering, 2nd Ed., University of California Press, Berkeley, CA), Roitt et al., 2001 (Immunology, 6th Ed., Mosby/Elsevier, Edinburgh), Roitt et al., 2001 (Roitt's Essential Immunology, 10th Ed., Blackwell Publishing, UK), and Janeway et al., 2005 (Immunobiology, 6^{th} Ed., Garland Science Publishing/Churchill Livingstone, New York), as well as to the general background art cited herein.

Unless indicated otherwise, all methods, steps, techniques and manipulations that are not specifically described in detail herein can be performed and have been performed in a manner known per se, as will be clear to the skilled person. Reference is, for example, again made to the standard handbooks and the general background art mentioned herein and to the further references cited therein; as well as to for example the following reviews: Presta 2006 (Adv. Drug Deliv. Rev., 58:640), Levin and Weiss 2006 (Mol. Biosyst., 2:49), Irving et al., 2001 (J. Immunol. Methods, 248:31), Schmitz et al., 2000 (Placenta 21 Suppl. A: S106), Gonzales et al., 2005 (Tumour Biol., 26:31), which describe techniques for protein engineering, such as affinity maturation and other techniques for improving the specificity and other desired properties of proteins such as immunoglobulins.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the technology described herein. Such equivalents are intended to be encompassed by the present technology.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

The term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acids or nucleotide sequences encoding the same, unless the context requires a more limited interpretation. Amino acid sequences are interpreted to mean a single amino acid or an unbranched sequence of two or more amino acids, depending on the context. Nucleotide sequences are interpreted to mean an unbranched sequence of 3 or more nucleotides.

It is understood that any reference to the amino acid sequences of the technology is meant to encompass post-translational modifications of these sequences occurring in mammalian cells such as CHO cells, including, but not limited to, N-glycosylation, O-glycosylation, deamidation, Asp isomerization/fragmentation, pyro-glutamate formation, removal of C-terminal lysine, and Met/Trp oxidation.

When a nucleotide sequence or amino acid sequence is said to “comprise” another nucleotide sequence or amino acid sequence, respectively, or to “essentially consist of” or to “consists of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein).

Amino acids are organic compounds that contain amino [a](—NH⁺₃) and carboxylate (—CO⁻₂) functional groups, along with a side chain (R group) specific to each amino acid. For instance, amino acids include those L-amino acids commonly found in naturally occurring proteins.

Amino acid residues will be indicated according to the standard three-letter or one-letter amino acid code. Reference is made to Table A-2 on page 48 of WO 08/020079. Examples of amino acids commonly found in proteins and represented in the genetic code are listed in Table 1 below. Other common amino acids (excluding those listed in Table 1 below) are described on the table on p. 624 of Pure & Appl. Chem., Vol. 56, No. 5, pp. 595-624, 1984, reproduced below as Table 2 for convenience.

TABLE 1
Common amino acids (IUPAC)

| 1-Letter | 3-Letter | |
|---|---|---|
| Code | Code | Name |
| A | Ala | Alanine |
| B | Asx | Aspartic acid or Asparagine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Uncommon or Unspecified |
| Y | Tyr | Tyrosine |
| Z | Glx | Glutamic acid or Glutamine |

TABLE 2
Further amino acids

| | | Structure of substance or of derived ion in the form |
|---|---|---|
| Trivial name | Symbol | predominating at neutral pH |
| β-Alanine | βAla | NH₃⁺—CH₂—CH₂—COO⁻ |
| Allysine | — | HCO—[CH₂]₃—CH(NH₃⁺)COO⁻ |
| Citrulline | Cit | NH₂—CO—NH—[CH₂]₃—CH(NH₃⁺)COO⁻ |
| Cystathionine | [symbol] | 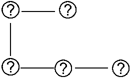 |
| Cysteic acid | Cya | ⁻O₃S—CH₂—CH(NH₃⁺)COO⁻ |
| Cystine | [symbol] | 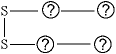 |
| Dopa | — | 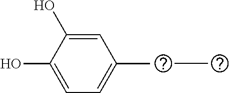 |
| Homocysteine | Hcy | HS—CH₂—CH₃—CH(NH₃⁺)COO⁻ |
| Homoserine | Hse | HO—CH₂—CH₃—CH(NH₃⁺COO⁻ |
| Homoserine lactone | Hsl | 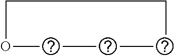 |
| Lanthionine | [symbol] | 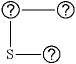 |
| Ornithine | Orn | NH₃⁺—[CH₂]₃—CH(NH₃⁺)COO⁻ |
| 5-Oxoproline | Glp | 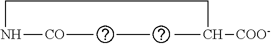 |
| Sarcosine | Sar | CH₃—NH₃⁺—CH₂—COO⁻ |
| Thyronine | — | 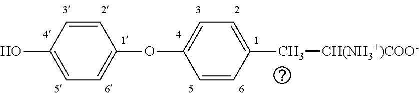 |
| Thyroxine | Thx | 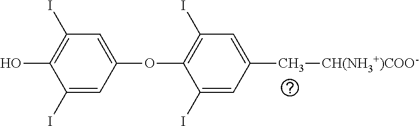 |

[symbol]

indicates data missing or illegible when filed

In the context of the present technology, the term “lysine” (K, Lys) refers to the natural, basic, 5 aliphatic α-amino acid comprising an α-amino group, an α-carboxylic acid group and a side chain lysyl ((CH₂)₄NH₂). For the purposes of this specification, lysine will refer to the biologically active enantiomer L-lysine, where the α-carbon is in the S configuration. The side chain of the lysine comprises a primary amine, which may serve as attachment point or conjugation site as described in the present technology.

The terms “protein”, “peptide”, “protein/peptide”, and “polypeptide” are used interchangeably throughout the present disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide and a protein; that polypeptides may be made by chemical synthesis or recombinant methods; and that proteins are generally made in vitro or in vivo by recombinant methods as known in the art.

By convention, the amide bond in the primary structure of polypeptides is in the order that the amino acids are written, in which the amine end (N-terminus) of a polypeptide is always on the left, while the acid end (C-terminus) is on the right.

Any amino acid sequence that contains post-translationally modified amino acids may be described as the amino acid sequence that is initially translated using the symbols shown in Table 1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition.

In the context of the present technology, the terms “specificity”, “binding specifically” or “specific binding” refer to the number of different target molecules, such as antigens, to which a particular binding unit can bind with sufficiently high affinity (see below). “Specificity”, “binding specifically” or “specific binding” are used interchangeably herein with “selectivity”, “binding selectively” or “selective binding”. Generally, binding units, such as binding ISVDs, specifically bind to their designated targets.

The specificity/selectivity of a binding unit can be determined based on affinity. The affinity denotes the strength or stability of a molecular interaction. The affinity is commonly given by the K_{D}, or dissociation constant, which has units of mol/litre (or M). The affinity can also be expressed as an association constant, K_{A}, which equals 1/K_{D} and has units of (mol/litre)⁻¹ (or M⁻¹).

The affinity is a measure for the binding strength between a moiety and a binding site on a target molecule: the lower the value of the K_{D}, the stronger the binding strength between a target molecule and a targeting moiety.

The K_{D}-value characterizes the strength of a molecular interaction also in a thermodynamic sense as it is related to the change of free energy (DG) of binding by the well-known relation DG=RT. In (K_{D}) (equivalently DG=-RT. In (K_{A})), where R equals the gas constant, T equals the absolute temperature and In denotes the natural logarithm.

The K_{D} may also be expressed as the ratio of the dissociation rate constant of a complex, denoted as k_{off} (or Kd), to the rate of its association, denoted kₒₙ (or Ka) (so that K_{D}=K_{off}/kₒₙ and K_{A}=Kₒₙ/K_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=In (2)/k_{off}. The off-rate may vary between 106 s⁻¹ (near irreversible complex with a t_{1/2} of multiple days) to 1 s⁻¹ (t_{1/2}=0.69 s).

The measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule or molecules. In such situation the measured affinity may be affected by the avidity of the interaction by the two molecules.

The dissociation constant (K_{D}) may be the actual or apparent dissociation constant, as will be clear to the skilled person. Methods for determining the K_{D} will be clear to the skilled person, and for example include the techniques mentioned below. In this respect, it will also be clear that it may not be possible to measure dissociation constants of more than 10⁻⁴ moles/litre or 10⁻³ moles/litre (e.g., of 10⁻² moles/litre). Optionally, as will also be clear to the skilled person, the (actual or apparent) K_{D} may be calculated on the basis of the (actual or apparent) association constant (K_{A}), by means of the relationship (K_{D}=1/K_{A}). K_{A}=1/K_{D}-->K_{A}= [AB]/[A]. [B].

The term “about” used in the context of the parameters or parameter ranges of the provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g., for a pH value of about pH 2.7, the error margin is 2.65-2.74. However, for the following parameters, the specific margins shall apply: a temperature specified in ° C. with no decimal place shall have an error margin of +1° C. (e.g., a temperature value of about 50° C. means 50° C.±1° C.); a time indicated in hours shall have an error margin of 0.1 hours irrespective of the decimal places (e.g., a time value of about 1.0 hours means 1.0 hours±0.1 hours; a time value of about 0.5 hours means 0.5 hours±0.1 hours).

In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence-compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0967284, EP 1085089, WO 00/55318, WO 00/78972, WO 98/49185 and GB 2357768. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence-compared to the first amino acid sequence-is considered as a difference at a single amino acid residue (position), i.e., as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the 3D structure, function, activity, or other biological properties of the polypeptide. Such conservative amino acid substitutions are well known in the art, for example from WO 04/037999, GB 335768, WO 98/49185, WO 00/46383, and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into 11e; Phe into Met, into Leu or into Tyr; Ser into Thr; Thr into Ser; Trp into Tyr; Tyr into Trp; and/or Phe into Val, into 11e or into Leu.

Amino acid sequences and nucleic acid sequences are said to be “exactly the same” if they have 100% sequence identity (as defined herein) over their entire length. When comparing two amino acid sequences, the term “amino acid difference” refers to an insertion, deletion or substitution of a single amino acid residue on a position of the first sequence, compared to the second sequence; it being understood that two amino acid sequences may contain one, two or more such amino acid differences.

According to the present description, “protein solubility” is a thermodynamic parameter defined as the concentration of protein in a saturated solution that is in equilibrium with a solid phase, either crystalline or amorphous, under a given set of conditions (see, e.g., Kramer R M. et al., “Toward a molecular understanding of protein solubility: increased negative surface charge correlates with increased solubility”, Biophys J., 2012, 10² (8): 1907-15).

### The Immunoglobulin Single Variable Domain (ISVD) of the Present Technology

The molecule of the present technology comprises, or alternatively, consists of, at least one immunoglobulin single variable domain (ISVD), as defined herein. For instance, the molecule of the present technology may comprise or, alternatively, consist of, a single ISVD. In other embodiments, the molecule comprises more than one ISVD, such as two, three, four, five, six or more ISVDs. The molecule of the present invention may also comprise at least one ISVD and one or more cargos attached to it, as described below.

The term “immunoglobulin single variable domain” (ISVD), interchangeably used with “single variable domain”, defines immunoglobulin molecules wherein the antigen binding site is present on, and formed by, a single immunoglobulin domain. This sets ISVDs apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an ISVD as, in these cases, binding to the respective epitope of an antigen would normally not occur by one single immunoglobulin domain but by a pair of associating immunoglobulin domains such as light and heavy chain variable domains, i.e., by a V_{H}-V_{L} pair of immunoglobulin domains, which jointly bind to an epitope of the respective antigen.

In contrast, generally, ISVDs are capable of specifically binding to an epitope of the antigen without pairing with an additional immunoglobulin variable domain. The binding site of an ISVD is formed by a single V_{H}, a single V_{HH} or single V_domain.

In the context of the present technology, the ISVD may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof. The ISVD can for example be a heavy chain ISVD, such as a V_{H}, V_{HH}, including a camelized V_{H} or humanized V_{HH}. In one embodiment, the ISVD is a V_{HH}, including a camelized V_{H} or humanized V_{HH}. Heavy chain ISVDs can be derived from a conventional four-chain antibody or from a heavy chain antibody.

For example, the ISVD may be a single domain antibody (or an amino acid sequence that is suitable for use as a single domain antibody), a “dAb” or dAb (or an amino acid sequence that is suitable for use as a dAb) or a NANOBODY® ISVD (as defined herein, and including but not limited to a V_{HH}); other single variable domains, or any suitable fragment of any one thereof.

Preferably, the ISVD is a V_{H}, a humanized V_{H}, a human V_{H}, a V_{HH}, a humanized V_{HH} or a camelized V_{H}. More preferably, the ISVD is a NANOBODY® ISVD (such as a V_{HH}, including a humanized V_{HH} or camelized V_{H}) or a suitable fragment thereof. NANOBODY® is a registered trademark from Ablynx N.V. “V_{HH} domains”, also known as V_{HH}'S, V_{HH} antibody fragments, and V_{HH} antibodies, have originally been described as the antigen binding immunoglobulin variable domain of “heavy chain antibodies”; i.e., of “antibodies devoid of light chains”, see Hamers-Casterman et al., Nature, 363:446-448, 1993. The term “V_{HH} domain” has been chosen in order to distinguish these variable domains from the heavy chain variable domains that are present in conventional 4-chain antibodies, which are referred to herein as “V_{H} domains”, and from the light chain variable domains that are present in conventional 4-chain antibodies, which are referred to herein as “V_{L} domains”. For a further description of V_{HH}'s, reference is made to the review article by Muyldermans (“Single domain camel antibodies: current status”, J Biotechnol., 2001, 74:277-302). V_{HH} domains can be obtained from heavy chain-only antibodies (HCAbs) that are circulating in Camelidae, see e.g., Muyldermans S., “A guide to: generation and design of nanobodies”, FEBS J., 2021, 288 (7): 2084-2102.

Typically, the generation of immunoglobulins involves the immunization of experimental animals, fusion of immunoglobulin producing cells to create hybridomas and screening for the desired specificities. Alternatively, immunoglobulins can be generated by screening of naïve, immune, or synthetic libraries, e.g., by phage display.

The generation of immunoglobulin sequences, such as V_{HHs}, has been described extensively in various publications, among which WO 94/04678, Hamers-Casterman et al. 1993 (“Naturally occurring antibodies devoid of light chains”, Nature, 363:446-448, 1993) and Muyldermans et al. 2001 (“Single domain camel antibodies: current status”, J Biotechnol., 2001, 74:277-302) can be exemplified. In these methods, camelids are immunized with the target antigen in order to induce an immune response against said target antigen. The repertoire of V_{HH}'s obtained from said immunization is further screened for V_{HHs} that bind (or not) a target antigen.

In the context of the present technology, immunoglobulin sequences of different origin may be used, comprising mouse, rat, rabbit, donkey, human and camelid immunoglobulin sequences. In the context of the present technology, fully human, humanized or chimeric sequences are also included. In the context of the present technology, camelid immunoglobulin sequences and humanized camelid immunoglobulin sequences, or camelized domain antibodies, e.g. camelized dAb as described by Ward et al. (Nature, 341:544, 1989) (see for example WO 94/04678 and Davies and Riechmann, “Camelising′ human antibody fragments: NMR studies on V_{H} domains”, Febs Lett., 339:285-290, 1994 and “Single antibody domains as small recognition units: design and in vitro antigen selection of camelized, human V_{H} domains with improved protein stability”, Prot. Eng., 1996, 9 (6): 531-537) are also included.

A “humanized V_{HH}” comprises an amino acid sequence that corresponds to the amino acid sequence of a naturally occurring V_{HH} domain, but that has been “humanized”, i.e., by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the further description herein and the prior art (e.g., WO 2008/020079). Again, it should be noted that such humanized V_{HH}'s can be obtained in any suitable manner known per se and thus are not strictly limited to polypeptides that have been obtained using a polypeptide that comprises a naturally occurring V_{HH} domain as a starting material. Preferably, if the ISVD of the present technology is a V_{HH}, the V_{HH} is a humanized V_{HH}.

A “camelized V_{H}” comprises an amino acid sequence that corresponds to the amino acid sequence of a naturally occurring V_{H} domain, but that has been “camelized”, i.e., by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g., WO 2008/020079). Such “camelizing” substitutions are usually inserted at amino acid positions that form and/or are present at the V_{H}-V_{L} interface, and/or at the so-called Camelidae hallmark residues, as defined herein (see for example WO 94/04678 and Davies and Riechmann, 1994 and 1996, supra). In one embodiment, the V_{H} sequence that is used as a starting material or starting point for generating or designing the camelized V_{H} is a V_{H} sequence from a mammal, or the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an ISVD sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

Also, as further described in paragraph q) on pages 58 and 59 of WO 2008/020079, the amino acid residues of an ISVD are numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 1999 (J. Immunol. Methods, 231 (1-2): 25-38; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering. This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application CDR sequences may also be described according to Kabat numbering with AbM CDR annotation (also referred to as “AbM numbering” or “AbM definition” in the present description), as described in Kontermann and Dübel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-10², and FR4 comprises the amino acid residues at positions 103-113.

Determination of CDR regions may also be done according to different methods. In the CDR determination according to Kabat, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-10², and FR4 of an ISVD comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are a suitable combination of immunoglobulin framework sequences or framework sequences that have been derived from immunoglobulin framework sequences, for example by humanization or camelization. For example, the framework sequences may be framework sequences derived from a light chain variable domain (e.g. a V_{L}-sequence) and/or from a heavy chain variable domain (e.g. a V_{H}-sequence or V_{HH} sequence). In one aspect, the framework sequences are either framework sequences that have been derived from a V_{HH}-sequence in which said framework sequences may optionally have been partially or fully humanized or are conventional V_{H} sequences that have been camelized (as defined herein).

In particular, the framework sequences present in the ISVD sequences referred to in the present technology may contain one or more of Hallmark residues (as defined herein), such that the ISVD sequence is a NANOBODY® ISVD, such as, e.g., a V_{HH}, including a humanized V_{HH} or camelized V_{H}. Some non-limiting examples of suitable combinations of such framework sequences will become clear from the further disclosure herein.

However, it should be noted that, in the context of the present technology, the origin of the ISVD sequence or the origin of the nucleotide sequence used to express it is not limited, nor as to the way that the ISVD sequence or nucleotide sequence is or has been generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template, e.g., DNA or RNA isolated from a cell, nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

In one embodiment, the at least one ISVD comprised in the molecule of the present technology is derived from a Nanobody® ISVD belonging to the so-called “V_{H}3 class”, i.e. a Nanobody® ISVDs with a high degree of sequence homology to human germline sequences of the V_{H}3 class such as DP-47, DP-51 or DP-29.

Generally, NANOBODY® ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Generally, a Nanobody® ISVD can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISVD can be an immunoglobulin sequence with the (general) structure

FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which:

one or more of the amino acid residues at positions 11, 37, 44, 45, 47, 83, 84, 103, 104 and 108 according to the Kabat numbering are chosen from the Hallmark residues mentioned in Table 3 below.

TABLE 3
Hallmark Residues in NANOBODY ® ISVDs (according to Kabat numbering)

| Position | Human V_{H}3 | Hallmark Residues |
|---|---|---|
| 11 | L, V; predominantly L | L, S, V, M, W, F, T, Q, E, A, R, G, K, Y, N, P, I; preferably |
| | | L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾;most preferably G⁽²⁾ or |
| | | Q⁽³⁾ |
| 45⁽⁸⁾ | L | L⁽²⁾, R⁽³⁾, P, H, F, G, Q, S, E, T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾ , L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; preferably |
| | | P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KOREL, KQREF, KEREG, KOREW or KQREG at positions 43-47. Alternatively, also sequences such as TERE (for example TEREL), TORE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KORE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

Thus, a NANOBODY® ISVD can be defined as an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are chosen from the Hallmark residues mentioned in Table 3.

In a further preferred embodiment, the ISVD of the present technology derives from an ISVD, such as from a heavy-chain ISVD, preferably from a NANOBODY® ISVD, which has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) may be Val or Leu, preferably Val; and/or the amino acid at position 89 (according to Kabat) may be preferably Val, Thr or Leu, preferably Leu; and/or the amino acid at position 110 (according to Kabat) may be preferably Thr, Lys or Gln, preferably Thr; and/or the amino acid at position 112 (according to Kabat) may be Ser, Lys or Gln, preferably Ser; and/or the ISVD may contain a C-terminal extension of 1-5 amino acids chosen from any naturally occurring amino acid.

In a preferred embodiment, the at least one ISVD comprised in the molecule of the present technology (the ISVD of the present technology) comprises a Gln (Q) at position 1 (Kabat), i.e., comprises a N-terminal Gln. Pyroglutamate (pE) or pyrrolidone carboxylate, is a cyclic amino acid found at the N-termini of some proteins and biological peptides. Formation occurs through the rearrangement of the originally synthesized glutamate or glutamine residues at this position. Both glutamine and glutamate at the N-termini has been shown to cyclize to pyroglutamate (pE) in vitro. Glutamate conversion to pyroglutamate occurs more slowly than from glutamine, see, e.g., Liu Y D. et al., “N-terminal glutamate to pyroglutamate conversion in vivo for human IgG2 antibodies”, J Biol Chem., 2011, 286 (13): 11211-7. Hence, in a preferred embodiment, the N-terminal of the at least one ISVD of the present technology comprises a pyroglutamate modification.

The at least one ISVD comprised in the molecule of the present technology is suitable for site-specific conjugation, preferably for primary amine site-specific conjugation, even more preferably, wherein the primary amine is present in the side chain of a lysine (lysine-specific conjugation), as defined below.

### Globular Three-Dimensional (3D) Structure

The ISVD of the present technology, as any ISVD, per definition, has a globular three-dimensional (3D) structure, i.e., it is or comprises a structured protein with a globular 3D structure. Globular proteins have approximately spherical shape. Nearly all globular proteins contain substantial numbers of α-helices and/or β-sheets folded into a compact structure that is stabilized by both polar and nonpolar interactions. The globular 3D structure forms naturally and often involves interactions mediated by the side chains of the amino acids. Most often, the hydrophobic amino acid side chains are buried, closely packed, in the interior of a globular protein, out of contact with water. Hydrophilic amino acid side chains lie on the surface of the globular proteins exposed to the water. Consequently, globular proteins are usually very soluble in aqueous solutions (from “Gene Expression: Translation of the Genetic Code”, Chang-Hui Shen, in Diagnostic Molecular Biology, 2019). In the context of the present technology, a protein or part of a protein with globular 3D structure can be defined as a protein or part of it which comprises at least one α-helix and/or at least one-sheet as part of its secondary structure. From a simple sequence of amino acids to its final 3D structure, a protein passes through four levels of structuring known as primary, secondary, tertiary, and quaternary. At the end of these stages the protein begins to fold up into a stable 3D structure that will allow it to fulfil its proper function. Hence, the amino acid sequence of a protein is known as the “primary structure” of that protein. The “secondary structure” can be defined as the arrangement of a polypeptide chain into more or less regular hydrogen-bonded structures, and it has two basic elements:
Alpha helix-spiral configuration of a polypeptide chain with 3.6 residues (amino acids) per turn. The helix may be left-handed or right-handed, and the latter is more common.
Beta strand (or beta-sheet)-two adjacent polypeptide strands that are bonded together. Two or more strands may interact to form a beta sheet.

Finally, the “tertiary structure” can be defined as the level of protein structure at which an entire polypeptide chain has folded into a 3D structure. In multi-chain proteins, the term tertiary structure applies to the individual chains. See Smith, A. D., et al., eds. 1997, Oxford Dictionary of Biochemistry and Molecular Biology, New York: Oxford University Press. The three-dimensional structure of a protein can be determined by techniques such as X-ray crystallography, nuclear magnetic resonance (NMR), cryo-electron microscopy (EM) or circular dichroism (CD). X-ray crystallography is a common technique used to determine 3D protein structure, but also NMR (suited for small proteins) and cryo-EM (suited for large proteins) can provide information about a protein's tertiary structure. Circular dichroism is an excellent method for rapidly evaluating the secondary structure, folding and binding properties of proteins, see, e.g., Jones, C. (“Circular dichroism of biopharmaceutical proteins in a quality-regulated environment”, J Pharm Biomed Anal., 2022, 219:114945). Because the CD spectra of proteins are so dependent on their conformation, CD can be used to estimate the structure of unknown proteins and monitor conformational changes due to temperature, mutations, heat, denaturants or binding interactions. For instance, α-helical proteins have negative bands at 222 nm and 208 nm and a positive band at 193 nm. Proteins with well-defined antiparallel β-pleated sheets (β-helices) have negative bands at 218 nm and positive bands at 195 nm, while disordered proteins have very low ellipticity above 210 nm and negative bands near 195 nm. See Greenfield N J., “Using circular dichroism spectra to estimate protein secondary structure”, Nat Protoc., 2006, 1 (6): 2876-90 for further details.

Hence, the ISVD of the present technology, as any ISVD, has a globular 3D tertiary structure. The structural architecture of ISVDs such as V_{HHs} comprises 2 β-sheets, one with 4 β-strands (A, B, D, and E) and the other with 5 β-strands (C, C′, C″, F and G), and CDRs form flexible antigen-binding loops between β-strands, see, e.g., FIG. 1 of Bingying Liu and Daiwen Yang, “Easily established and multifunctional synthetic nanobody libraries as research tools”, Int. J. Mol. Sci. 2022, 23, 1482. Hence, the ISVD of the present technology, as any ISVD, comprises a globular 3D structure which comprises 2 β-sheets (preferably one with 4 β-strands and another one with 5 β-strands) and three flexible loops between β-strands, where the antigen-binding domains (CDRs) are located.

The globular 3D structure of ISVDs allows the engineering of site- and stereospecific-conjugation sites or attachment points, as described in detail in this specification. The presence of at least one α-helix and/or at least one β-sheet in a certain polypeptide or protein can be determined by known techniques, as explained above, such as, e.g., CD.

As shown in the examples, the structure integrity of the ISVDs can also be monitored by assessing its thermal stability (e.g., by determining the melting temperature, Tm), see, e.g., Examples 1 and 3. If the Tm of the ISVD is not significantly decreased as compared with the Tm of its ISVD precursor, it can be concluded that the 3D structure of the resulting ISVD is maintained (i.e., the resulting ISVD has a globular 3D structure, as the precursor does). Preferably, the Tm of the resulting ISVD is not decreased more than 10° C., preferably is not decreased more than 8° C., or 6° C., even more preferably is not decreased more than 5° C., or more than 4° C., even more preferably is not decreased more than 3° C., or more than 2° C., or more than 1° C. Preferably, the Tm of the resulting ISVD is about the same Tm than the Tm of the precursor, or 1, 2, 3, 4, 5, 6, 7, 8, 9, or 10° C. lower, preferably at most 5° C. lower than the Tm of the ISVD precursor.

### Solubility

The ISVD of the present technology is soluble. In the context of the present technology, a soluble ISVD means that the ISVD has a solubility of 10 mg/ml or more, preferably of 20 mg/mL, preferably of 50 mg/ml or more, and even more preferably of 100 mg/ml or more, measured in water or a suitable buffer or solvent (e.g., an aqueous solution, or a physiological buffer, such as a buffer which is amenable for parenteral administration) at room temperature (RT). In a preferred embodiment, the solubility of the ISVD is measured in water or in a suitable buffer at RT, more preferably in a buffer such as citrate buffer (e.g., citrate buffer 5 mM) or PBS, at pH 7.0 or 7.4, at RT. Other preferred suitable buffers which are suitable for measuring the solubility of the ISVD are Dulbecco's phosphate buffered saline (DPBS, which is a s a balanced salt solution containing potassium chloride, monobasic potassium phosphate, sodium chloride, and dibasic sodium phosphate), preferably pH 7.0 or 7.4, at RT. Other preferred suitable buffers which are suitable for measuring the solubility of the protein-based carrier building block are Dulbecco's phosphate buffered saline (DPBS, which is a s a balanced salt solution containing potassium chloride, monobasic potassium phosphate, sodium chloride, and dibasic sodium phosphate), preferably pH 7.0 or 7.4, at RT, or histidine buffer at pH 6.5, at RT (comprising histidine (10 mM to 100 mM, such as 10 mM), sucrose (1% to 10%, such as 10%) and, optionally, Tween 80 (0.001% to 1%, such as 0.01%)), or phosphate buffer pH 7.0, at RT (comprising NaH₂PO₄/Na₂HPO₄ (10 and 50 mM, such as 10 mM), sodium chloride (NaCl) (100-150 mM, such as 130 mM NaCl) and, optionally, Tween 80 (0.001% to 1%, such as 0.01%)).

The skilled person is aware of methods to measure the solubility of a protein solution. For instance, the supplementary material of Kramer R M. et al. (“Toward a molecular understanding of protein solubility: increased negative surface charge correlates with increased solubility”, Biophys J., 2012, 102 (8): 1907-15) describes solubility measurements of folded proteins.

Additionally or alternatively, solubility measurements can be performed as follows. The protein solution (e.g., in citrate buffer 5 mM, pH 7.0, or in PBS pH 7.4, or in water, or in any of the suitable buffers described above) is concentrated by ultrafiltration (e.g., via tangential flow filtration (TFF)) until some cloudiness appears in the solution. Then, the solution is spined at high speed or 0.22 μm filtered to remove any non-soluble material, and the OD280 of the supernatant is measured. Using the molar extinction coefficient of the specific protein, the protein concentration of the supernatant (and, thus, the concentration of the protein in a saturated solution that is in equilibrium with a solid phase, i.e., the protein solubility) is obtained.

### ISVD Precursor

The at least one ISVD comprised in the molecule of the present technology may be generated from another ISVD, which is selected as starting point for developing the ISVD of the present technology (the so-called “ISVD precursor”). An ISVD precursor is an ISVD which sequence is suitable to be modified, and/or is intended to be modified (e.g., by point mutations or by addition and/or removal of one or more lysines to/from its N- and/or C-terminus) to generate the ISVD of the present technology. In addition, if necessary, the ISVD precursor is modified so that it incorporates one or more attachment points or conjugation sites as described herein. The ISVD precursor has a sequence identity of at least 60%, such as at least 70%, or at least 75%, preferably of at least 80% with the ISVD derived from it. For instance, the ISVD precursor has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with the ISVD derived from it. For instance, the ISVD precursor may share the whole amino acid sequence with ISVD derived from it with the exception of at least one, such as one, two, three, four, five, six, seven, eight, nine, ten, eleven, twelve, thirteen, fourteen, fifteen, twenty or more amino acids.

For instance, the ISVD precursor is modified by at least one point mutation, wherein the point mutation comprises the substitution of one amino acid which is not lysine in the sequence of the ISVD precursor by a lysine, or wherein the ISVD precursor is modified by at least one point mutation, wherein the point mutation comprises the substitution of a lysine by an amino acid which is not lysine. For instance, the ISVD precursor is modified by the addition of one lysine to its N- and/or C-terminus. In addition, the ISVD precursor may be modified by a point mutation of the amino acid at position 1 (Kabat), wherein the point mutation consists of the replacement of the amino acid at position 1 (Kabat) by a Gln (Q), preferably wherein the point mutation consists of the replacement of the N-terminal glutamic acid (E) of the ISVD precursor by a glutamine (Q).

It is preferred that the ISVD precursor is modified, as described herein, without significantly affecting its desirable structural and/or functional properties, such as its primary, secondary or tertiary structure and/or conformation and/or its advantageous performance essentially resulting from its binding properties, as defined herein.

The ISVD precursor is preferably a V_{HH}, such as a NANOBODY® ISVD or a suitable fragment thereof, more preferably a humanized V_{HH}, such as a humanized NANOBODY® ISVD or a suitable fragment thereof. The resulting ISVD should be soluble and have a globular 3D structure. Hence, in one embodiment, the ISVD of the present technology is soluble and has a globular 3D structure.

In a preferred embodiment, the ISVD of the present technology has a sequence identity of at least 80% with a V_{HH} (such as a humanized V_{HH} or camelized V_{H}), e.g., with its V_{HH} precursor. For instance, the ISVD—has a sequence identity of at least 60%, or at least 70%, or 80%, or at least 85%, such as at least 90%, such as at least 91%, such as at least 92%, such as at least 93%, such as at least 94%, such as at least 95%, such as at least 96%, such as at least 97%, such as at least 98%, such as at least 99%, or more with a V_{HH}, e.g., its V_{HH} precursor. For instance, the ISVD may share the whole amino acid sequence with its V_{HH} precursor with the exception of at least one, such as one, two, three, four, five, six, seven, eight, nine, ten, fifteen, eighteen, twenty, twenty-five, thirty or more amino acids, which are different in the ISVD.

In one embodiment, the ISVD precursor does not comprise any lysine in its amino acid sequence. Preferably, the ISVD precursor does not comprise any primary amine in its sequence. Hence, in a preferred embodiment, the ISVD precursor does not comprise any lysine in its sequence and, moreover, does not comprise a N-terminal primary amine, e.g., because the N-terminal comprises a pyroglutamate modification. Hence, the present technology is also directed to an ISVD which does not comprise any lysine in its amino acid sequence, preferably which does not comprise any primary amine in its sequence.

### Functionality of the ISVDs

In a preferred embodiment, the ISVD of the present technology is a functional ISVD, i.e., it shows specific binding toward its antigen(s) (target(s)). Hence, in a preferred embodiment, the ISVD precursor and the ISVD derived from it show specific binding towards the same antigen(s)/target(s), preferably with similar affinities. For instance, the K_{D} value of the interaction between the ISVD of the present technology and a certain target(s) is not significantly increased (i.e., worsened in binding) in comparison with the K_{D} value of the interaction between the ISVD precursor and the same target(s). “Not significantly increased” in this specific context may mean that the K_{D} value is not increased (i.e. worsened in binding) by more than one order of magnitude.

In one embodiment, the ISVD of the present technology may not specifically bind to any human protein. Hence, in this embodiment, if the ISVD shows any interaction with one or more human proteins, such interaction is characterized by low specificity and/or low affinity, as defined herein.

For instance, the ISVD of the present technology, in this particular embodiment, may not specifically bind crystallizable fragment (Fc) receptors (FcRs), Fc-binding proteins or Fc-sensors. For instance, the ISVD of the present technology may not specifically bind C-type lectin receptors (CLRs). All antibodies possess two functional domains-one that confers antigen specificity, known as the antigen-binding fragment (Fab), and another that drives antibody function, known as the crystallizable fragment (Fc). The specific effector functions that are triggered by antibodies are determined by the receptors to which the antibody Fc domain binds and the specific innate immune cells on which these FcRs are expressed. These sensors include both classical FcRs and non-classical C-type lectin receptors (CLRs), see Lu, L. et al., “Beyond binding: antibody effector functions in infectious diseases”, Nat Rev Immunol, 2018, 18, 46-61. Table 1 of Lu, L. et al provides non-limiting examples of Fc domain sensors (e.g., Fcγ or FcRn) to which the ISVD of the present technology do not specifically bind.

Consequently, the ISVD of the present technology, in this particular embodiment, may not show effector functions of conventional antibodies mediated by the Fc domain. In another embodiment, the ISVD and/or the molecule of the present technology may not specifically bind crystallizable fragment (Fc) receptors (FcRs), Fc-binding proteins or Fc-sensors. For instance, in this embodiment, the ISVD and/or the molecule of the present technology may not specifically bind C-type lectin receptors (CLRs). Hence, in one embodiment, none of the components comprised in the molecule of the present technology (e.g., at least one ISVD and/or at least one cargo attached or conjugated to it) specifically bind crystallizable fragment (Fc) receptors (FcRs), Fc-binding proteins, Fc-sensors and/or CLRs. In another embodiment, the ISVD and/or the molecule of the present technology may not show effector functions of conventional antibodies mediated by the Fc domain, i.e., none of the components comprised in the molecule of the present technology show effector functions of conventional antibodies mediated by the Fc domain. In one embodiment, the molecule of the present technology does not include conventional V_{H}-V_{L} pairing/interaction and/or does not include C_{L}-C_{H}1 pairing such as C_{L}-C_{H}1 binding disulphide bridges.

In another embodiment, the ISVD of the present technology may not specifically bind the variable domain of the light chain (V_{L}) and/or the variable domain of the heavy chain (V_{H}) of an antibody, such as the V_{L} and/or the V_{H} of a monoclonal antibody (mAb). In another embodiment, the ISVD of the present technology may not specifically bind the first constant domain of the heavy chain (C_{H}1) of an antibody, such as the C_{H}1 of a mAb. In another embodiment, the ISVD of the present technology may not specifically bind the constant domain of the light chain (Ct) of an antibody, such as the CL of a mAb. In another embodiment, the ISVD of the present technology may not specifically bind the third constant domain of the heavy chain (C_{H}3) of an antibody, such as the C_{H}3 of a mAb. In another embodiment, the ISVD of the present technology may not specifically bind the second constant domain of the heavy chain (C_{H}2) of an antibody, such as the C_{H}2 of a mAb. In one embodiment, the molecule and/or ISVD of the present technology is not a Fab fragment from an antibody, such as from a mAb. In one embodiment, the molecule and/or ISVD of the present technology is not a C_{H}, preferably is not a C_{H}1 fragment from an antibody, such as from a mAb. In the molecule and/or ISVD of the present technology is not an antibody, such as a mAb, is not a Fc fragment, or a Fv fragment.

In one embodiment, the ISVD of the present technology does also not specifically bind to any non-protein molecule (including biomolecules, such as nucleic acids (e.g., DNA, RNA), lipids or glycans), e.g., to any non-protein human molecule, including biomolecules, such as human nucleic acids (e.g., human DNA, human RNA), human lipids (e.g., such as phosphatidylserine (PS)) or human glycans, e.g., human glycoplipids. In particular, the ISVD of the present technology may also not specifically bind any non-protein molecule (including biomolecules, such as nucleic acids (e.g., DNA, RNA), lipids (e.g., such as phosphatidylserine (PS)) or glycans).

In another embodiment, the ISVD of the present technology may also not specifically bind to any (non-human) molecule (including biomolecules)

In another embodiment, the ISVD of the present technology may not specifically bind to any human cell. If the ISVD shows any interaction with one or more human cells, such interaction may be characterized by low specificity and/or low affinity, as defined herein. The lack of binding to any human cell can for example be assessed with the “cell binding assay” as described below (see, e.g., Hunter S A and Cochran J R, “Cell-binding assays for determining the affinity of protein-protein interactions: technologies and considerations”, Methods Enzymol., 2016, 580:21-44).

In another embodiment, the ISVD of the present technology may not specifically bind to any molecule, including human biomolecules and non-human biomolecules (e.g., non-human proteins, nucleic acids such as DNA and/or RNA, lipids (e.g., such as phosphatidylserine (PS)) or glycans), or may bind to any biomolecule, including human biomolecules and non-human biomolecules (e.g., non-human proteins, DNA, RNA, lipids (e.g., such as phosphatidylserine (PS)) or glycans) with a K_{D} (K_{D} value) greater than 5×10⁻⁴ mol/litre, as described herein. For instance, the ISVD of the present technology may not specifically bind to any bacterial biomolecule (e.g., bacterial proteins, nucleic acids such as DNA, RNA, lipids (e.g., such as phosphatidylserine (PS)) or glycans), or may bind to any bacterial biomolecule with a K_{D} (K_{D} value) greater than 5×10⁻⁴ mol/litre, as described herein. For instance, the ISVD of the present technology may not specifically bind to any viral molecule (e.g., viral proteins, viral nucleic acids such as viral DNA, RNA, viral lipids (e.g., such as phosphatidylserine (PS)) or viral glycans), or may bind to any viral molecule with a K_{D} (K_{D} value) greater than 5×10⁻⁴ mol/litre, as described herein. For instance, the ISVD of the present technology may not specifically bind to any plant molecule (e.g., bacterial proteins, bacterial nucleic acids such as bacterial DNA, RNA, bacterial lipids (e.g., such as phosphatidylserine (PS)) or bacterial glycans), or may bind to any bacterial molecule with a K_{D} (K_{D} value) greater than 5×10⁻⁴ mol/litre, as described herein. For instance, the ISVD of the present technology may not specifically bind to any mammalian molecule (e.g., mammalian proteins, mammalian nucleic acids such as DNA, RNA, lipids (e.g., such as phosphatidylserine (PS)) or glycans), or may bind to any mammalian molecule with a K_{D} (K_{D} value) greater than 5×10⁻⁴ mol/litre, as described herein.

In the context of the present technology, the term “biomolecule” or “biological molecule” refers to molecules present in organisms, including microorganisms and/or viruses that are essential to one or more typically biological processes, such as cell division, morphogenesis, or development. Biomolecules are the building blocks of life and perform important functions in living organisms. Biomolecules include the primary metabolites which are large macromolecules such as proteins, carbohydrates (glycans), lipids (e.g., such as PS), and nucleic acids (such as DNA, RNA), as well as small molecules such as vitamins and hormones. The four major types of biomolecules are carbohydrates (glycans), lipids, nucleic acids, and proteins.

In a further embodiment, the ISVD of the present technology may not specifically bind any non-human protein and/or any non-protein molecule when at least one cargo is conjugated to the at least one attachment point or conjugation site on the ISVD.

The affinity of a molecular interaction between two molecules can be measured via different techniques known per se, such as the well-known surface plasmon resonance (SPR) biosensor technique (see for example Ober et al. 2001, Intern. Immunology 13:1551-1559, in particular section “Surface plasmon resonance (SPR) experiments” starting on p. 1552, which describes conditions for measuring the affinity of a molecular interaction between two molecules). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a Cytiva lifesciences company, Uppsala, Sweden and Piscataway, NJ). For further descriptions, see Jonsson et al. (1993, Ann. Biol. Clin. 51:19-26), Jonsson et al. (1991 Biotechniques 11:620-627), Johnsson et al. (1995, J. Mol. Recognit. 8:125-131), and Johnnson et al. (1991, Anal. Biochem. 198:268-277). For instance, the affinity (K_{D}) of a molecular interaction between two molecules can be determined via SPR on a ProteOn XPR36 instrument (Bio-Rad Laboratories). The experiment can be performed at 25° C., and as assay buffer PBS pH7.4 containing 0.005% Tween 20 (Bio-Rad Laboratories) can be used. Targets such as human proteins or non-protein molecules, such as DNA, RNA, lipids (e.g., such as phosphatidylserine (PS)) or glycans can be immobilized onto different ligand lanes from a GLC sensorchip (Bio-Rad Laboratories), e.g., with the ProteOn Amine Coupling Kit (Bio-Rad Laboratories) according to the manufacturer's instructions. The ISVD of the present technology can be captured on the target immobilized ligand lanes. One ligand lane can serve as a reference surface and no ISVD was captured on the surface. Different concentrations (e.g., ranging from 300 nM to 1.2 nM) diluted in running buffer can be flowed over the respective ISVDs and reference surface in multi-cycle kinetics for 2 minutes, followed by a constant flow of the assay buffer for 15 minutes. Between the different injections, the surfaces can be regenerated with 3 M MgCl2 (Cytiva). Several buffer blanks can be injected for double referencing. Data can be analyzed, e.g., with the ProteOn Manager 3.1.0 software (Bio-Rad Laboratories). The kinetic rate constants (ka and kd) can be calculated by fitting the sensorgrams via the Langmuir 1:1 interaction ligand binding model. The equilibrium dissociation constant K_{D} can be calculated as the kd/ka ratio. See also “Application Note: kinetic and epitope characterization of influenza antiviral targets using Alto™ digital surface plasmon resonance”, SinoBiological, Nicoya, nicoyalife.com/wp-content/uploads/2023/02/characterization-of-Influenza-using-Alto.pdf.

Another well-known biosensor technique to determine affinities of biomolecular interactions is bio-layer interferometry (BLI) (see for example Abdiche et al. 2008, Anal. Biochem. 377:209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al., “Characterizing high-affinity antigen/antibody complexes by kinetic- and equilibrium-based methods”, Anal. Biochem., 2004, 328:35-43), using the KinExA® platform (Sapidyne Instruments Inc, Boise, USA). The “term “KinExA”, as used herein, refers to a solution-based method to measure true equilibrium binding affinity and kinetics of unmodified molecules. Equilibrated solutions of a binding unit/target complex, such as an antibody/antigen complex, are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

Further, the GYROLAB® immunoassay system provides a platform for automated bioanalysis and rapid sample turnaround (Fraley et al., “The Gyrolab™ immunoassay system: a platform for automated bioanalysis and rapid sample turnaround”, Bioanalysis 2013, 5:1765-74).

Further, the affinity of a molecular interaction between two molecules (e.g., between two proteins) can be measured using flow cytometry to analyze ligand binding to antigens such as proteins, lipids (e.g., phosphatidylserine (PS)), sugars (glycans), etc. presented on the surface of a cell (“cell binding assay”). The skilled person is familiar with cell-binding assays to determine the affinity of a certain soluble molecule (such as the molecule and/or ISVD of the present technology) and a binding partner present on the surface of a cell, such as a human cell. For instance, Hunter S. A. and Cochran J. R. (“Cell-binding assays for determining the affinity of protein-protein interactions: technologies and considerations”, Methods Enzymol. 2016, 580:21-44), present a practical guide for measuring binding events between soluble ligands and binding partners expressed on the surface of, inter alia, mammalian cells. For instance, as shown in the examples, the cell binding assay can be carried out as follows:
a. Adding a fixed number of human cells to a 96-well V-bottom plate (e.g., 50 μl human cell suspension (5E+04/96-well), or to tubes, such as Eppendorf tubes, in cold fluorescence-activated cell sorting (FACS) buffer (e.g., consisting of D-PBS, 2% heat inactivated fetal bovine serum (HI FBS) and 0.05% Sodium Azide).
b. Performing a washing step;
c. Adding the soluble molecule, e.g., the molecule of the present technology, preferably marked, such as with a fluorescent label or an epitope tag, and incubate for a certain amount of time, until the reaction has come to equilibrium, generally a number of hours, e.g., for about 3h, at low temperature, typically 4° C. while shaking;
d. Evaluate the binding of the soluble molecule to the human cells by flow cytometry, e.g., by FACS.

In order to calculate the K_{D} of the binding between a soluble molecule, e.g., the molecule of the present technology, and a human cell, the soluble molecule in step c. above may be added to each well/tube at varying concentrations, spanning two orders of magnitude above and below the anticipated K_{D}. Binding values can be determined from the average signal value (e.g., average fluorescence value) of each sample, plotting the fraction bound vs. ligand concentration (log scale) and fitting a sigmoidal curve using nonlinear regression analysis. The K_{D} value can be derived from the ligand concentration at half the fraction bound.

The skilled person is able to determine whether a molecule is able to specifically bind human proteins, as defined in the context of the present technology. For instance, the skilled person may make use of commercially available protein arrays to determine the binding affinity of a certain molecule (protein) towards human proteins. For instance, the skilled person may make use of the commercially available Proteome Profiler™ Antibody Arrays, which allows for the semiquantitative measurement of more than 100 proteins in a single sample. Alternatively or additionally, the skilled person may make use of, for example, HuProt™ assay, such as the version v4.0, which consists of >21,000 unique human proteins, isoform variants, and protein fragments-covering 16,794 unique genes. This includes 15,889 of the 19,613 canonical human proteins described in the Human Protein Atlas, with broad coverage across protein subclasses.

Similarly, the skilled person is able to determine whether a molecule is able to specifically bind a non-human protein, such as a bacterial or viral protein. For instance, the skilled person may make use of protein-binding assays to determine the binding affinity of a certain molecule (e.g., a protein) towards non-human (such as bacterial or viral) proteins. Similarly, the skilled person is able to determine whether a molecule (e.g., a protein) is able to specifically bind a non-protein molecule, e.g., a human non-protein molecule, such as human DNA, human RNA, human lipids or human glycans, see, e.g., Campanero-Rhodes M A et al., “Microarray strategies for exploring bacterial surface glycans and their interactions with glycan-binding proteins”, Front Microbiol. 2020, 10:2909. For instance, as described above, the binding affinity of a molecular interaction between two molecules (such as two proteins, or a protein and a non-protein molecule) can be measured by SPR. SPR allows for the determination of the K_{D} of a potential interaction between two molecules, as described in detail above.

As it will be evident to the skilled person, the at least one ISVD comprised in the molecule of the present technology may show non-specific binding with one or more human proteins (and/or with one or more non-human proteins, and/or with one or more non-protein molecules, such as human non-protein molecules, as described above). This is because there may be molecular forces between the at least one ISVD of the present technology and one or more human proteins (and/or one or more non-human proteins, and/or one or more non-protein molecules, such as human non-protein molecules, as described above), e.g., in the form of hydrophobic interactions, hydrogen bonding, Van der Waals interactions, and other nonspecific interactions. Hence, if this happens, the at least one ISVD comprised in the molecule of the present technology may non-specifically bind to one or more human proteins (and/or to one or more non-human proteins, and/or to one or more non-protein molecules, such as human non-protein molecules, if this is the case, as described above). In the context of the present technology, any K_{D} value greater than 5×10⁻⁴ mol/litre (or any K_{A} value lower than 2×10³ litres/mol) is generally considered to represent “non-specific binding”. Hence, in one embodiment, the ISVD of the present technology may bind to any human protein (or non-human protein, if this is the case, as explained above) with a K_{D} greater than 5×10⁻⁴ mol/litre (or with a K_{A} value lower than 2×10³ litres/mol), such as with a K_{D} greater than 5.5×10 4 mol/litre (or with a K_{A} value lower than 1.8×10³ litres/mol), or with a K_{D} greater than 6×10 4 mol/litre (or with a K_{A} value lower than 1.7×10³ litres/mol). In addition, in the context of the present technology, in a preferred embodiment, the ISVD may bind to any non-protein molecule, such as to any human non-protein molecule (e.g., DNA, RNA, lipids, glycans) with a K_{D} greater than 5×10⁻⁴ mol/litre (or with a K_{A} value lower than 2×10³ litres/mol), such as with a K_{D} greater than 5.5×10⁻⁴ mol/litre (or with a K_{A} value lower than 1.8×10³ litres/mol), or with a K_{D} greater than 6×10⁻⁴ mol/litre (or with a K_{A} value lower than 1.7×10³ litres/mol). In the context of the present technology, this binding affinity is considered to be “non-specific binding”.

In one embodiment, the ISVD of the present technology is not derived from the crystallizable fragment of an antibody (Fc, which contains two C_{H}2 and two C_{H}3 domains) such as the Fc fragment of a monoclonal antibody (mAb). In another embodiment, the ISVD of the present technology is not derived from the C_{H}2 and/or the C_{H}3 domains of the Fc fragment. In another embodiment, the ISVD of the present technology is not derived from a C_{H}1 and/or the CL domains comprised in the antigen-binding fragment (Fab) of an antibody, such as the C_{H}1 and/or the CL domains comprised in the Fab of a mAb. In one embodiment, the molecule of the present technology is not (or is not derived from) a crystallizable fragment (Fc) of an antibody, such as a mAb. In another embodiment, the molecule of the present technology is not (or is not derived from) the Fab of an antibody, such as a mAb.

In one embodiment, the molecule of the present technology does not comprise V_{H}-V_{L} pairs or, e.g., it does not comprise at least one V_{H} and at least one V_{L} which interact (are bound to) with each other, such as in an antibody. In another embodiment, the molecule of the present technology does not comprise C_{L}-C_{H}1 conjugates, e.g., it does not comprise at least one CL and at least one C_{H}1 which are linked to each other, e.g., through a disulphide bridge.

### Attachment Points or Conjugation Sites

As mentioned above, the ISVD present in the molecule of the present technology has at least one “attachment point” (also referred to as “conjugation site” in the present disclosure), preferably at a solvent-accessible position, as defined further below. Preferably, the at least one ISVD comprises more than one attachment points or conjugation sites, preferably at solvent-accessible positions. In a preferred embodiment, the ISVD of the present technology comprises at least two attachment points or conjugation sites. In another embodiment, the ISVD comprises three conjugation sites or more, such as six, seven, eight, or nine conjugation sites, or more. For instance, ISVD may have two, three, four, five, six, seven, eight, nine, ten conjugation sites or more.

In another embodiment, alternatively or additionally, if there is more than one conjugation site, the conjugation sites are spatially distant from each other (spatially separated from each other). The skilled person will appreciate that the minimal distance between conjugation sites will be dictated by the nature of the cargos (and linkers, if used) which are to be attached or conjugated to the attachment points or conjugation sites in the ISVD. For larger cargos (e.g., ISVDs), the minimal distance can still be kept small when used in combination with long linkers, which add the needed flexibility and the envisaged target binding. A short distance between conjugation sites, combined with short linkers, if any, will likely limit the target binding (or other activity) of larger cargos, and result in restricted engagement (e.g., increased cell specificity). In addition, the solubility of the molecule may be decreased (i.e., the molecule may be more prone to aggregation). On the other hand, if the cargos to be attached are rather small (e.g., radioactive isotopes), the minimal distance can be kept small even in the absence of linkers. Hence, the skilled person will be able to select the location of the specific conjugation sites and the length and flexibility of the linkers, if any, depending on the nature of the cargos which are to be attached or conjugated to the ISVD.

In the context of the present technology, a “conjugation site” or “attachment point” is preferably a primary amine, e.g., the primary amine in the side chain of a lysine, preferably located at a solvent-accessible position in the ISVD, or the N-terminal primary amine in the ISVD. Preferably, in the context of the present technology, a “conjugation site” or “attachment point” is the primary amine present in the side chain of a lysine, i.e., of a natural amino acid which is a lysine. Of course, the ISVD of the present technology may comprise other conjugation sites or attachment points (for site-specific and/or stochastic conjugation) which are not primary amines. In one embodiment, the ISVD of the present technology comprises only primary amines as conjugation sites or attachment points. In one embodiment, the ISVD of the present technology comprises only primary amines in the side chain of lysines as conjugation sites or attachment points. Hence, in a preferred embodiment, the “conjugation site(s)” or “attachment point(s)” present in the ISVD of the present technology is (are) primary amine(s) present in the side chain of one or more lysines. In one embodiment, the ISVD of the present technology comprises a N-terminal comprises a pyroglutamate modification, i.e., does not comprise the N-terminal primary amine.

In the context of the present technology, the “C-terminal or N-terminal reactive group of the ISVD” refers to the —COOH and —NH₂ reactive groups present in the C- and N-terminal amino acid of the ISVD. The ISVD may not have a free C- and/or N-terminal end (e.g., because the ISVD is C- and/or N-terminal linked to a cargo molecule as defined herein, or to another ISVD, or to another peptide or protein, or because the N-terminal is acetylated, or has been subjected to pyroglutamation, see below, or because the C-terminal is amidated, etc.). If the N-terminal of the ISVD is not free (e.g., because the ISVD is N-terminal linked to another ISVD, or to another peptide or protein, or because the N-terminal is acetylated, or has been subjected to pyroglutamation), then it is not suitable as attachment point or conjugation site as defined herein.

The at least one conjugation site or attachment point present in the ISVD of the present technology may be already present in the ISVD precursor (e.g., the —NH₂ group in the side chain of a lysine present in the ISVD precursor, preferably at a solvent-accessible position, or the N-terminal primary amine) or may be engineered. Preferably, at least one or more of the attachment points or conjugation sites of the ISVD are engineered. In the context of the present technology, an “engineered” attachment point or conjugation site, or an “engineered” lysine, means a conjugation site or attachment point, or a lysine, which is present in the ISVD, but which was not present in its precursor at the same or corresponding position. For instance, the ISVD may be modified to introduce one or more attachment points or conjugation sites (i.e., one or more lysines), as described in detail below. A non-limiting example of an engineered attachment point or conjugation site is a primary amine present in the side chain of a lysine in the ISVD which lysine was not present at the same or equivalent position in the ISVD precursor. For instance, if the ISVD has an arginine at a certain position X (which is preferably a solvent-accessible position) in the ISVD precursor, and that arginine is mutated to a lysine in the ISVD, the —NH₂ group of that lysine would be an engineered attachment point or conjugation site. For instance, if a lysine is added at the N- or C-terminal of the ISVD precursor, the —NH₂ group present in the side chain of that newly added lysine would be an engineered attachment point or conjugation site.

Hence, in a preferred embodiment, the ISVD of the present technology has at least one conjugation site or attachment point which is an engineered attachment point or conjugation site, i.e., it was not present in the ISVD precursor at the same or corresponding position. In another preferred embodiment, all of the conjugation sites or attachment points present in the ISVD of the present technology are engineered attachment points or conjugation sites, i.e., they were not present in the ISVD precursor at the same or corresponding position. In one embodiment, the ISVD of the present technology has two or more engineered attachment points or conjugation sites, such as three, four, five, six, seven, eight, nine, ten or more engineered attachment points or conjugation sites.

As used herein a residue position in one polypeptide sequence “corresponds to” a residue position in another polypeptide sequence if it exists in an equivalent position in the polypeptide sequence, as indicated, e.g., by primary sequence homology or functional equivalence or Kabat numbering. A corresponding position may be identified by alignment of the two polypeptide sequences. The alignment used to identify a corresponding position or corresponding region may be obtained using a conventional alignment algorithm such as Blast (Altschul et al., “Basic local alignment search tool”, J Mol Biol., 1990, 215 (3): 403-10).

Thus, the at least one conjugation site present in the ISVD of the present technology is a primary amine. For instance, the conjugation site may be a free or capped (protected) primary amine, preferably located at a solvent-accessible position.

Hence, the ISVD of the present technology is designed as a “carrier” or “delivery” moiety, with at least one attachment point or conjugation site at desired position(s) in the sequence of the ISVD. The at least one “attachment point” or “conjugation site” is preferably a primary amine comprised in the ISVD of the present technology, e.g., the N-terminal primary amine and/or a primary amine on the side chain of a Lys. The ISVD of the present technology preferably comprises at least two attachment points or conjugation sites, or more, as described below (e.g., two lysines, or more, or one lysine (or more) and the N-terminal primary amine), for conjugation or attachment of cargos, as defined in detail below. Suitable cargos include proteins, peptides, toxic payloads, nucleic acids, oligonucleotides, fluorophores, glycans, chelators for/and radio-isotopes, half-life extending moieties, e.g., polyethylene glycol (PEG) molecules and/or HSA-based or binding moieties, vitamins (such as biotin or folate), etc. Specific non-limiting examples of suitable cargos are depicted below in the present description.

### Addition of a C- or N-lysine

For instance, the conjugation sites may be generated by adding, in the ISVD precursor, one or more C- or N-terminal lysines. Preferably, if present, the one or more terminal Lys is added at the C-terminus of the ISVD precursor. Hence, in one embodiment, the ISVD of the present technology comprises an engineered lysine located at the C-terminus of the ISVD's amino acid sequence.

Finally, as described above, the conjugation sites may be generated by combinations of the above mechanisms, e.g., the at least one conjugation site can be obtained by performing point mutations, as described above, and/or by adding a C- and/or N-terminal lysine, as described above.

Solvent-accessible positions

As described above, the at least one conjugation site or attachment point (i.e., the at least one lysine or primary amine) present in the ISVD of the present technology, if any, is preferably located at a solvent-accessible position in the ISVD.

The skilled person is able to identify “solvent-accessible positions” in an ISVD. This can be performed in silico by means of computer modelling. For instance, the skilled person can make use of readily available software tools such as MAESTRO (Schrödinger, LLC, New York, NY, 2021), a multi-agent prediction system, based on statistical scoring functions (SSFs) and different machine learning approaches, see, e.g., Laimer et al. BMC Bioinformatics (2015) 16:116. In addition, the skilled person can also make use of readily available software tools such as YASARA (www.yasara.org), for identifying at least potential solvent-accessible positions for the at least one conjugation site of the ISVD. With the help of in silico tools such as MAESTRO or YASARA, the skilled person is able to identify solvent-accessible positions that are potentially suitable for engineering conjugations sites as defined above. Hence, with the help of tools such as MAESTRO or YASARA, potentially suitable conjugation sites are identified. An example of how to identify solvent-accessible positions that are potentially suitable for engineering conjugations sites as defined above is provided herein. For instance, one ISVD is selected as starting point for developing the ISVD of the present technology (the ISVD precursor). Using, e.g., MAESTRO, residues in the ISVD precursor with a Solvent-Accessible Surface Area (SASA) greater than or equal to, e.g., 27 Å² (square angstrom) can be considered to be solvent-accessible. The stability (ΔG in solvent) of the mutation of each of the identified residues (e.g., to a lysine residue) can then be calculated, see, e.g., Laimer J. et al, “MAESTRO—multi agent stability prediction upon point mutations”, BMC Bioinformatics, 2015, 16:116, for further details. Destabilizing mutations (e.g., mutations for which the calculated ΔG in solvent is higher) are generally not further considered as potential positions for conjugation sites or attachment points. Hence, once potentially suitable conjugation sites are identified with the help of tools such as MAESTRO or YASARA, the stability (ΔG in solvent) of the mutation of each of the identified residues (e.g., to a lysine residue) is calculated. Those residues with lower calculated ΔG in solvent would be preferably further selected as potential positions for conjugation sites or attachment points. For instance, ΔG values in the range of −20 to +5 can be considered as non-destabilizing mutations. The skilled person will understand that the ΔG value for each of the mutations of the identified residues may vary depending on the specific protein and/or the specific mutations considered. The skilled person will also understand that the preferred mutations are those whose ΔG values are the lowest. Depending on these ΔG values, the number of conjugation sites and the type of cargo that will be conjugated, the skilled person will further select certain positions over others among the ones initially identified as potentially solvent-accessible with the help of tools such as MAESTRO or YASARA.

Alternatively or additionally, the skilled person can use hydrogen/deuterium exchange mass spectrometry (HDX-MS) to determine at least potential solvent-accessible positions in a protein. HDX-MS reports on the local chemical environment and solvent accessibility of the protein backbone by monitoring the exchange of peptide bond amide protons with the deuterons of a D20 solvent. The rate of hydrogen-deuterium exchange is dependent on the solvent accessibility and folded state of the protein (see Englander S W. et al., “Hydrogen exchange: the modern legacy of Linderstrøm-Lang”, Protein Sci., 1997, 6 (5): 1101-9).

If the identified solvent-accessible position is to be occupied by a certain amino acid with a reactive group in its side chain, (e.g., by a lysine), the in silico modelling (e.g., with MAESTRO) will also take into account the potential interactions of the reactive group of that amino acid with other reactive groups present in the side chain of other amino acids present in the ISVD.

Additionally or alternatively, the “solvent-accessible positions” can be identified and/or verified empirically. For instance, the “solvent-accessible positions” theoretically identified using available in silico software tools such as MAESTRO, as described above, may preferably be empirically confirmed by manufacturability. Formulation and process stability of potential ISVD candidates help narrow down lead candidates at an early stage, prior to large-scale manufacturing (see the examples and also, e.g., Ramachander, R., Rathore, N. (2013), “Molecule and manufacturability assessment leading to robust commercial formulation for therapeutic proteins” in: Kolhe, P., Shah, M., Rathore, N. (eds) Sterile Product Development, AAPS Advances in the Pharmaceutical Sciences Series, vol 6. Springer, New York, NY). Hence, once potential suitable solvent-accessible positions have been theoretically identified in the ISVD precursor, expression levels, conjugation efficiency, formulation, quality control, solubility, process stability, etc., of the resulting ISVD should preferably be evaluated. Solvent-accessible positions which lead to ISVD excelling in expression yield, manufacturability, solubility and/or stability are preferred.

For instance, once suitable solvent-accessible positions have been theoretically identified in the ISVD precursor, protein expression of the selected variants (i.e., the resulting ISVDs with amino acid(s) bearing the conjugation site(s) in the theoretically-selected solvent-accessible position(s)) may take place. In this step it can be asserted whether the introduction of the specific amino acids at the theoretically-identified solvent accessible positions (e.g., point mutations, addition of amino acids at the N- and/or C-terminal of the protein) has a negative impact on, e.g., the synthesis, expression levels, conjugation efficiency, binding affinity, etc. of each specific variant. Possible changes in 3D structure could be assessed, for example, by CD (circular dichroism) spectrum analysis, as described in detail above. In addition, the stability of the resulting variants can also be confirmed with a Thermal Shift Assay. This assay detects protein melting temperatures (Tm) and can thus be used to check protein stability. It can be used to characterize the stability/folding of a protein's 3D structure. SYPRO® Orange is a naturally quenched dye that interacts with the hydrophobic core of proteins which becomes visible following thermal denaturation. As a result, the temperature in the middle of the thermal denaturation process is labelled as melting temperature Tm. This is a way of assessing the stability of the resulting ISVDs, see the examples for more details.

In addition, “model cargos” can be attached or conjugated to the selected variants, in order to quantify the extent of conjugation (conjugation efficiency), i.e., to ascertain whether the resulting ISVD with the conjugation sites at the selected solvent-accessible positions will in practice be suitable for the attachment or conjugation of the desired cargos. A “model cargo” may be any molecule with a molecular weight higher than, e.g., 100 Da. For instance, if a potential conjugation site is a primary amine, a “model cargo” may be a labelled ester, such as a biotin-labelled ester, e.g., a N-hydroxysuccinimide (NHS)—long chain (LC)—Biotin ester, such as EZ-Link Sulfo-NHS-LC Biotin (see Example 4), which can react efficiently with primary amines to form stable, irreversible amide bonds. For instance, if the conjugation of the “model cargo(s)” results in a stable conjugate (ISVD with one or more model cargos conjugated to it), with an acceptable extent of conjugation (to be decided on a case-by-case basis, for example≥90% conjugation efficiency, such as 90% conjugation efficiency, or 95% conjugation efficiency or more, or 97% conjugation efficiency or more, or 99% conjugation efficiency or more), allowing a standard PK in vivo, preserving its globular 3D structure and the conjugation status in vivo, etc., those solvent-accessible positions should be preferred for cargo conjugation, and conjugation of the desired cargo(s) may take place, see also the examples below.

In a preferred embodiment, the solvent accessible position is selected from the following positions (according to Kabat numbering): 5, 7, 10, 12, 13, 19, 43, 44, 45, 61, 64, 70, 71, 73, 75, 83, 85, 105 and/or 108, preferably from the following positions (according to Kabat numbering): 19, 43, 45, 64, 71, 73, 75, 83, 105 and/or 108, and more preferably from the following positions (according to Kabat numbering): 45, 73, and/or 108. In one embodiment, the solvent accessible position is selected from the following positions (according to Kabat numbering): 5, 7, 10, 12, 13, 44, 45, 61, 70, 71, 73, 85 and/or 108. In one embodiment, the solvent accessible position is not one or more of the following positions (according to Kabat numbering): 19, 43, 64, 75, 83, 97 and 105.

### Point Mutations

In one embodiment, the at least one conjugation site present in the ISVD may be generated by introducing specific point mutations at solvent-accessible positions in the peptide sequence of the ISVD precursor. For instance, point mutations may be introduced at solvent-accessible positions in the ISVD precursor in order to generate the ISVD comprised in the molecule of the present technology, which comprises at least one conjugation site or attachment point at defined solvent-accessible positions, as described herein.

For instance, the conjugation sites may be generated by mutating specific amino acids preferably at solvent-accessible positions of an ISVD precursor to lysine (“Lys-mutations”). The preferred solvent accessible positions are: 5, 7, 10, 12, 13, 19, 43, 44, 45, 61, 64, 70, 71, 73, 75, 83, 85, 105 and/or 108 (according to Kabat), more preferably, 19, 43, 45, 64, 71, 73, 75, 83, 105 and/or 108, such as at positions 43 and/or 45, or positions 45, 108 and/or 73, preferably 45. In one embodiment, the solvent accessible position is selected from the following positions (according to Kabat numbering): 5, 7, 10, 12, 13, 44, 45, 61, 70, 71, 73, 85 and/or 108. Preferably the solvent accessible position is selected from the following positions (according to Kabat numbering): 45, 108 and 73. In one embodiment, the solvent accessible position is not one or more of the following positions (according to Kabat numbering): 19, 43, 64, 75, 83, 97 and 105.

Additionally or alternatively, the ISVD precursor may be modified by adding one or more Lys at the N- and/or C-terminal of the protein sequence, to introduce at least one conjugation site or attachment point, as described herein, to generate the ISVD of the present technology.

In another embodiment, the at least one conjugation site may be already present preferably at solvent-accessible positions in the ISVD precursor, and there is no need of generating it.

This is the case for the primary amine at the N-terminal of the ISVD, or the primary amine in the side chain of, e.g., a lysine preferably already present at a solvent-accessible position in the ISVD precursor, e.g., at positions 43, 64, 75 and/or 83 (according to Kabat).

If the ISVD comprises more than one conjugation sites, these can be generated by introducing, e.g., specific point mutations preferably at solvent-accessible positions in the peptide sequence of the ISVD precursor to lysine (“Lys-mutations”). Additionally or alternatively, other suitable conjugation sites or attachment points may be already present preferably at solvent-accessible positions in the ISVD precursor, i.e., there is no need of generating these conjugation sites by introducing, e.g., specific point mutations to lysine and/or adding one or more lysines at the N- and/or C-terminal of the ISVD precursor. The skilled person will decide on the number and position of the attachment point(s) or conjugation site(s) based on the ISVD and the cargo(s) to be attached to it, directly or by means of a linker, as described herein. This way, site-specific conjugation on one or more lysines, locates at the desired positions, can be achieved.

As described in detail above, preferably, the point mutations are non-destabilizing point mutations. Stability of mutants can be calculated with different methods which predict the impact of mutations on protein stability, e.g., based on artificial intelligence (AI). For instance, stability of mutants can be calculated with MAESTRO, as defined above and explained in detail in the examples, and can also be confirmed empirically by manufacturability (including but not limited to expression level and stability assessment, as described above).

In a preferred embodiment, the point mutations are mutations of amino acids preferably located at solvent-accessible positions in the ISVD precursor to lysines. In another embodiment, the point mutation consists of the replacement of a residue preferably in a solvent-accessible position of the ISVD precursor by a lysine, wherein the residue in the ISVD precursor is selected from: Arginine (Arg), Asparagine (Asn) and/or Glutamine (Gln). In another embodiment, the point mutations are selected from the following (numbering according to Kabat): N73K, R83K, R19K, Q105K, Q108K, R71K and/or R45K. Preferably, the point mutations are selected from the following (numbering according to Kabat): N73K, Q108K and/or R45K. In one embodiment, the point mutation consists of the replacement of a residue preferably in a solvent-accessible position of the ISVD precursor by a lysine, wherein the residue in the ISVD precursor is preferably not Alanine (Ala).

In another embodiment, the point mutations are mutations of preferably solvent-accessible lysines in the ISVD precursor to an amino acid different from lysine. Preferably, the amino acid different or distinct from lysine is selected from: glutamine, histidine, arginine, alanine, or glutamate, more preferably glutamine, histidine, arginine, or glutamate, even more preferably arginine. In one embodiment, the amino acid different or distinct from lysine is not alanine. In a preferred embodiment, the ISVD of the present technology comprises at least one, preferably at least two amino acids distinct from lysine at at least one, preferably two of positions 43, 64 and/or 75 (according to Kabat), preferably wherein the at least one, preferably at least two amino acids distinct from lysine are selected from glutamine, histidine, arginine, alanine, or glutamate, more preferably at least one amino acid selected from glutamine, histidine, arginine, or glutamate, even more preferably at least one arginine. In one embodiment, the amino acid different or distinct from lysine is not alanine. In a preferred embodiment, the ISVD of the present technology comprises at least one, preferably at least two amino acids distinct from lysine at at least one, preferably two of positions 43, 64, 75 and/or 83 (according to Kabat), preferably wherein the at least one, preferably at least two amino acids distinct from lysine are selected from glutamine, histidine, arginine, alanine, or glutamate, more preferably at least one amino acid selected from glutamine, histidine, arginine, or glutamate, even more preferably at least one arginine. In one embodiment, the amino acid different or distinct from lysine is not alanine. In another preferred embodiment, the ISVD of the present technology comprises amino acids distinct from lysine at positions 43, 64, 75 and 83 (according to Kabat), preferably wherein the amino acids distinct from lysine are selected from glutamine, histidine, arginine, alanine, or glutamate, more preferably at least one amino acid selected from glutamine, histidine, arginine, or glutamate, even more preferably at least one arginine. In one embodiment, the amino acid different or distinct from lysine is not alanine. In another preferred embodiment, the ISVD of the present technology comprises amino acids distinct from lysine at positions 45, 73 and 108 (according to Kabat), preferably wherein the amino acids distinct from lysine are selected from glutamine, histidine, arginine, alanine, or glutamate, more preferably at least one amino acid selected from glutamine, histidine, arginine, or glutamate, even more preferably at least one arginine. In one embodiment, the amino acid different or distinct from lysine is not alanine.

In one embodiment, the at least one ISVD comprised in the molecule of the present technology comprises at least one engineered lysine at at least one solvent accessible position. As described above, an engineered lysine in the ISVD of the present technology refers to a lysine which is present in the ISVD of the present technology, but which was not present in its precursor at the same or corresponding position. In a preferred embodiment, the at least one engineered lysine (or at least one attachment point or conjugation site) is located in a region of the ISVD which corresponds to the FRs of the ISVD, i.e., the engineered lysine is not located in the CDRs of the ISVD. In another preferred embodiment, the engineered lysine (or at least one attachment point or conjugation site) is located in the ISVD at a position different from position 43, 64, 75 and/or 83 (according to Kabat). Hence, in this embodiment, the at least one ISVD comprised in the molecule of the present technology comprises at least one engineered lysine, preferably more, such as 2, 3, 4, 5, 6, 7, 8, 9 or more engineered lysines, preferably at solvent accessible positions. Preferably, at least one, such as 1, 2, 3, 4, 5, 6, 7, 8, 9, or more, and more preferably all of the engineered lysine present in the ISVD are located in the FRs, preferably at solvent accessible positions in the FRs, i.e., are not located in the CDRs.

In one embodiment, the ISVD of the present technology does not comprise a C-terminal Lys-tag, wherein the Lys-tag comprises three lysines. Hence, in one embodiment, the ISVD of the present technology does not comprise three C-terminal lysines in its sequence.

In another embodiment, all of the lysines comprised in the at least one ISVD comprised in the molecule of the present technology are engineered lysines, preferably located in the ISVD at a position different from position 43, 64 and/or 75, even more preferably located in the ISVD at a position different from position 43, 64, 75 and/or 83 (according to Kabat), preferably at solvent accessible positions. Hence, in one embodiment, the ISVD of the present technology comprises at least one lysine at at least one solvent accessible position, wherein the solvent accessible position is not located at positions 43, 64, 75 and/or 83 (according to Kabat), preferably wherein the solvent accessible position is not located at positions 43, 64, 75, 83 and/or 97 (according to Kabat), even more preferably wherein the solvent accessible position is not located at positions 19, 43, 64, 75, 83, 97 and/or 105 (according to Kabat).

In another embodiment, the ISVD of the present technology comprises at least one lysine at at least one of the following positions: 5, 7, 10, 12, 13, 19, 43, 44, 45, 54, 61, 64, 70, 71, 73, 75, 83, 85, 95, 105 and/or 108 (according to Kabat). Preferably, if the sequence of the ISVD of the present technology comprises at least one lysine, the at least one lysine is located at at least one of the following positions: 5, 7, 10, 12, 13, 19, 43, 44, 45, 61, 64, 70, 71, 73, 75, 83, 85, 105 and/or 108 (according to Kabat). In a preferred embodiment, the ISVD of the present technology does not comprises lysines at positions different from 5, 7, 10, 12, 13, 19, 43, 44, 45, 54, 64, 61, 70, 71, 73, 75, 83, 85, 95, 105 and/or 108 (according to Kabat). Hence, in a preferred embodiment, all of the lysines comprised in the sequence of the ISVD of the present technology are located within the following positions: 5, 7, 10, 12, 13, 19, 43, 44, 45, 54, 61, 64, 70, 71, 73, 75, 83, 85, 95, 105 and/or 108 (according to Kabat). In another preferred embodiment, all of the lysines comprised in the sequence of the ISVD of the present technology are located within the following positions: 45, 73 and/or 108 (according to Kabat). In one embodiment, the ISVD of the present technology does not comprise a lysine at any of the following positions: 19, 43, 64, 75, 83, 97 and 105 (according to Kabat). As shown in the examples, performing point mutations at solvent accessible positions, wherein the amino acids in the ISVD precursor are replaced or substituted by lysine, does not compromise the melting temperature™ and the affinity of the ISVDs.

Hence, in one preferred embodiment, the ISVD of the present technology comprises at least one lysine at at least one of the following positions: 5, 7, 10, 12, 13, 19, 43, 44, 45, 61, 64, 70, 71, 73, 75, 83, 85, 105 and/or 108 (according to Kabat) and does not comprise any lysine at a position different from positions: 5, 7, 10, 12, 13, 19, 43, 44, 45, 61, 64, 70, 71, 73, 75, 83, 85, 105 and/or 108 (according to Kabat). In another embodiment, the ISVD of the present technology comprises at least one lysine at at least one of the following positions: 5, 7, 10, 12, 13, 44, 45, 61, 70, 71, 73, 85 and/or 108 (according to Kabat) and does not comprise any lysine at the following positions (according to Kabat): 19, 43, 64, 75, 83, 97 and 105.

In another preferred embodiment, the ISVD of the present technology comprises at least one lysine at at least one of the following positions: 19, 43, 45, 64, 71, 73, 75, 83, 105 and/or 108 (according to Kabat) and does not comprise any lysine at a position different from positions: 19, 43, 45, 64, 71, 73, 75, 83, 105 and/or 108 (according to Kabat). In another preferred embodiment, the ISVD of the present technology comprises at least one lysine at at least one of the following positions: 45, 71, 73 and/or 108 (according to Kabat) and does not comprise any lysine at the following positions (according to Kabat): 19, 43, 64, 75, 83, 97 and 105.

In another preferred embodiment, the ISVD of the present technology comprises at least one lysine at at least one of the following positions: 5, 7, 10, 12, 13, 44, 45, 61, 70, 71, 73, 85, 105 and/or 108 (according to Kabat) and does not comprise any lysine at a position different from positions: 5, 7, 10, 12, 13, 44, 45, 61, 70, 71, 73, 85, 105 and/or 108 (according to Kabat). In another preferred embodiment, the ISVD of the present technology comprises at least one lysine at at least one of the following positions: 73 and/or 83 (according to Kabat) and does not comprise any lysine at a position different from positions: 73 and/or 83 (according to Kabat).

In another preferred embodiment, the ISVD of the present technology comprises at least one lysine at at least one of the following positions: 19, 73 and/or 83 (according to Kabat) and does not comprise any lysine at a position different from positions: 19, 73 and/or 83 (according to Kabat).

In another preferred embodiment, the ISVD of the present technology comprises at least one lysine at at least one of the following positions: 19, 83 and/or 105 (according to Kabat) and does not comprise any lysine at a position different from positions: 19, 83 and/or 105 (according to Kabat).

In another preferred embodiment, the ISVD of the present technology comprises at least one lysine at at least one of the following positions: 19, 73, 83 and/or 105 (according to Kabat) and does not comprise any lysine at a position different from positions: 19, 73, 83 and/or 105 (according to Kabat).

In another preferred embodiment, the ISVD of the present technology comprises at least one lysine at at least one of the following positions: 19, 71, 83 and/or 105 (according to Kabat) and does not comprise any lysine at a position different from positions: 19, 71, 83 and/or 105 (according to Kabat).

In another preferred embodiment, the ISVD of the present technology comprises at least one lysine at at least one of the following positions: 19, 45, 73, 83 and/or 105 (according to Kabat) and does not comprise any lysine at a position different from positions: 19, 45, 73, 83 and/or 105 (according to Kabat).

In another preferred embodiment, the ISVD of the present technology comprises at least one lysine at at least one of the following positions: 19, 71, 73, 83 and/or 105 (according to Kabat) and does not comprise any lysine at a position different from positions: 19, 71, 73, 83 and/or 105 (according to Kabat).

In another preferred embodiment, the ISVD of the present technology comprises at least one lysine at at least one of the following positions: 19, 45, 71, 73, 83 and/or 105 (according to Kabat) and does not comprise any lysine at a position different from positions: 19, 45, 71, 73, 83 and/or 105 (according to Kabat).

In another preferred embodiment, the ISVD of the present technology comprises at least one lysine at at least one of the following positions: 19, 45, 71, 73, 83 and/or 105 (according to Kabat) and does not comprise any lysine at a position different from positions: 19, 45, 71, 73, 83 and/or 105 (according to Kabat).

In another preferred embodiment, the ISVD of the present technology comprises at least one lysine at at least one of the following positions: 45 and/or 108 (according to Kabat) and does not comprise any lysine at a position different from positions: 45 and/or 108 (according to Kabat).

In another preferred embodiment, the ISVD of the present technology comprises at least one lysine at at least one of the following positions: 45 and/or 73 (according to Kabat) and does not comprise any lysine at a position different from positions: 45 and/or 73 (according to Kabat).

In another preferred embodiment, the ISVD of the present technology comprises at least one lysine at at least one of the following positions: 5 and/or 105 (according to Kabat) and does not comprise any lysine at a position different from positions: 5 and/or 105 (according to Kabat).

In another preferred embodiment, the ISVD of the present technology comprises at least one lysine at at least one of the following positions: 13 and/or 45 (according to Kabat) and does not comprise any lysine at a position different from positions: 13 and/or 45 (according to Kabat).

In another preferred embodiment, the ISVD of the present technology comprises at least one lysine at at least one of the following positions: 45, 73 and/or 108 (according to Kabat) and does not comprise any lysine at a position different from positions: 45, 73 and/or 108 (according to Kabat).

In one embodiment, the ISVD of the present technology does not comprise a lysine at the following positions: 19 and 105 (according to Kabat).

In one embodiment, the ISVD of the present technology does not comprise a lysine at the following positions: 43, 64, 75 and 83 (according to Kabat). In another embodiment, the ISVD of the present technology does not comprise a lysine at the following positions: 43, 64, 75, 83 and 97 (according to Kabat). In another embodiment, the ISVD of the present technology does not comprise a lysine at the following positions: 19, 43, 64, 75, 83 and 105 (according to Kabat). In another embodiment, the ISVD of the present technology does not comprise a lysine at the following positions: 19 and 105 (according to Kabat).

According to the IMGT numbering system, positions 19, 43, 64, 75, 83, 97 and 105 (according to Kabat) would be positions 20, 48, 72, 84, 95, 109 and 120 (according to IMGT), respectively.

In another embodiment, the ISVD of the present technology comprises a single engineered lysine, preferably at a solvent accessible position. In another embodiment, all of the lysines comprised in the ISVD of the present technology are engineered lysines. In another embodiment, the ISVD of the present technology comprises only one lysine in its sequence, and it is an engineered lysine, preferably located at the C-terminus of the ISVD's amino acid sequence or at one of the following positions: 73, 45, 105 or 108 (Kabat). In one embodiment, the ISVD of the present technology comprises only one lysine in its sequence, added to the C-terminus of the ISVD's amino acid sequence. In another embodiment, the ISVD of the present technology comprises only one lysine in its sequence, and it is an engineered lysine, preferably located at the C-terminus of the ISVD's amino acid sequence and/or at one of the following positions: 73, 45 or 108 (Kabat).

In another embodiment, the ISVD of the present technology comprises at least one engineered lysine, preferably at a solvent accessible position, and at least one lysine at at least one of the following positions: 43, 64, 75 and/or 83 (according to Kabat). For instance, the ISVD of the present technology may comprise one, two, three or four lysines at positions 43, 64, 75 and/or 83 (according to Kabat) and at least one engineered lysine, preferably at a solvent accessible position, preferably more than one engineered lysine, preferably at at least one position selected from: 5, 7, 10, 12, 13, 19, 44, 45, 61, 70, 71, 73, 83, 85, 105 and/or 108 (according to Kabat). For instance, the ISVD of the present technology may comprise one, two, three or four lysines at positions 43, 64, 75 and/or 83 (according to Kabat) and at least one engineered lysine, preferably at a solvent accessible position, preferably more than one engineered lysine, preferably at at least one position selected from: 5, 7, 10, 12, 13, 19, 44, 45, 61, 70, 71, 73, 83, 85, 105 and/or 108 (according to Kabat) and/or is added to the C-terminus.

In another preferred embodiment, the ISVD of the present technology comprises a single lysine, wherein the single lysine is located at positions 43, 64, 75 or 83 (according to Kabat). In another embodiment, the ISVD of the present technology comprises a single lysine at position 43, 64 or 75, and the other two positions (e.g., 43 and 64, or 43 and 75, or 64 and 75) are occupied by amino acids which are not Lys. For instance, the ISVD of the present technology comprises one Lys at position 43, 64 or 75 and at least another engineered lysine at a position different from positions 43, 64 or 75, preferably at a solvent accessible position, even more preferably at at least one position selected 5, 7, 10, 12, 13, 19, 44, 45, 61, 70, 71, 73, 83, 85, 105 and/or 108 (according to Kabat), more preferably selected from 19, 45, 71, 73, 83, 105 and/or 108, even more preferably selected from 45, 73, and/or 108, even more preferably selected from 45, 73, and/or 108, such as at position 45.

The ISVD of the present technology may also comprise at least two lysines at positions 43, 64, 75 and/or 83. The ISVD of the present technology may also comprise at least two lysines at positions 43, 64, 75 and/or 83 and at least another engineered lysine at a position different from positions 43, 64, 75 and/or 83, preferably at a solvent accessible position, more preferably at at least one position selected from 5, 7, 10, 12, 13, 19, 44, 45, 61, 70, 71, 73, 83, 85, 105 and/or 108, even more preferably selected from 19, 45, 71, 73, 105 and/or 108, even more preferably selected from 45, 73, and/or 108, such as at position 45. In an embodiment, the ISVD of the present technology comprises lysines at position 43, 64 and 75 and at least another engineered lysine at a solvent accessible position, more preferably at at least one position selected from 5, 7, 10, 12, 13, 19, 44, 45, 61, 70, 71, 73, 83, 85, 105 and/or 108, even more preferably selected from 19, 45, 71, 73, 83, 105 and/or 108.

In another embodiment, the ISVD of the present technology comprises up to 10 lysines, such as 1, 2, 3, 4, 5, 6, 7, 8, 9, or 10 lysines, wherein at least one lysine is at position 43, 64 or 75 and up to nine engineered lysines (e.g. 1, 2, 3, 4, 5, 6, 7, 8, or 8) are at a position different from positions 43, 64 or 75, preferably at a solvent accessible position, more preferably at a position selected from 5, 7, 10, 12, 13, 19, 44, 45, 61, 70, 71, 73, 83, 85, 105 and/or 108, even more preferably selected from 19, 45, 71, 73, 83, 105 and/or 108 and/or the C-terminus. In another embodiment, the ISVD of the present technology comprises up to 10 lysines, such as 1, 2, 3, 4, 5, 6, 7, 8, 9, or 10 lysines, wherein three lysines are at position 43, 64 or 75 and up to seven engineered lysines are at a solvent accessible position, more preferably at up to seven positions selected from 5, 7, 10, 12, 13, 19, 44, 45, 61, 70, 71, 73, 83, 85, 105 and/or 108, even more preferably selected from 19, 45, 71, 73, 83, 105 and/or 108 and/or the C-terminus.

In a specific embodiment, the ISVD of the present technology comprises five lysines the following positions: 43, 64, 73, 75, and 83 (according to Kabat) and does not comprise any lysine at a position different from positions: 43, 64, 73, 75, and 83 (according to Kabat). In a specific embodiment, the ISVD of the present technology comprises six lysines the following positions: 19, 43, 64, 73, 75, and 83 (according to Kabat) and does not comprise any lysine at a position different from positions: 19, 43, 64, 73, 75, and 83 according to Kabat). In a specific embodiment, the ISVD of the present technology comprises seven lysines the following positions: 19, 43, 64, 73, 75, 83 and 105 (according to Kabat) and does not comprise any lysine at a position different from positions: 19, 43, 64, 73, 75, 83 and 105 according to Kabat. In a specific embodiment, the ISVD of the present technology comprises seven lysines the following positions: 19, 43, 64, 71, 75, 83 and 105 (according to Kabat) and does not comprise any lysine at a position different from positions: 19, 43, 64, 71, 75, 83 and 105 according to Kabat. In a specific embodiment, the ISVD of the present technology comprises seven lysines the following positions: 19, 43, 45, 64, 73, 75, 83 and 105 (according to Kabat) and does not comprise any lysine at a position different from positions: 19, 43, 45, 64, 73, 75, 83 and 105 according to Kabat. In a specific embodiment, the ISVD of the present technology comprises eight lysines the following positions: 19, 43, 64, 71, 73, 75, 83 and 105 (according to Kabat) and does not comprise any lysine at a position different from positions: 19, 43, 64, 71, 73, 75, 83 and 105 according to Kabat. In a specific embodiment, the ISVD of the present technology comprises nine lysines the following positions: 19, 43, 45, 64, 71, 73, 75, 83 and 105 (according to Kabat) and does not comprise any lysine at a position different from positions: 19, 43, 45, 64, 71, 73, 75, 83 and 105 according to Kabat.

In another preferred embodiment, the at least one ISVD comprised in the molecule of the present technology comprises a single lysine in its amino acid sequence, wherein the single lysine is located at at least one position selected from: 45, 73, and 108 (according to Kabat).

As shown in the examples, the inventors have surprisingly found that performing point mutations at positions 45, 73, and 108 (according to Kabat) does not compromise the Tm and affinity of the resulting ISVDs. Preferably, the at least one ISVD comprises a single lysine at two positions selected from: 45, 73, and 108 (according to Kabat) and comprises an amino acid selected from glutamine, histidine, arginine, alanine, and glutamate, preferably arginine, at the other position of 45, 73, and 108. Preferably, the at least one ISVD comprises a lysine at two positions selected from: 45, 73, and 108 (according to Kabat).

In another embodiment, the at least one ISVD comprised in the molecule of the present technology comprises two lysines in its amino acid sequence, wherein the two lysine are located at at least two positions selected from: 45, 73, and 108 (according to Kabat). In another embodiment, the at least one ISVD comprised in the molecule of the present technology comprises two lysines in its amino acid sequence, wherein one lysine is located at at 45, 73, and 108 (according to Kabat) and another lysine is located at a position selected from 5, 7, 10, 12, 13, 19, 44, 61, 70, 71, 83, 85, and/or 105, such as 5, 13 or 105. Preferably, the at least one ISVD comprises two lysines at two positions selected from 45, 73, and 108 (according to Kabat) and comprises an amino acid selected from glutamine, histidine, arginine, alanine, and glutamate, preferably arginine, at the other position of 45, 73, and 108.

Hence, in a preferred embodiment, the ISVD of the present technology comprises two lysines at the following positions: 45 and 108 (according to Kabat) and does not comprise any lysine at a position different from positions: 45 and 108 (according to Kabat).

In another preferred embodiment, the ISVD of the present technology comprises two lysines at the following positions: 45 and 73 (according to Kabat) and does not comprise any lysine at a position different from positions: 45 and 73 (according to Kabat).

In another preferred embodiment, the ISVD of the present technology comprises two lysines at the following positions: 13 and 45 (according to Kabat) and does not comprise any lysine at a position different from positions: 13 and 45 (according to Kabat).

In another preferred embodiment, the ISVD of the present technology comprises at the following positions: 5 and 105 (according to Kabat) and does not comprise any lysine at a position different from positions: 5 and 105 (according to Kabat).

In another embodiment, the at least one ISVD comprised in the molecule of the present technology comprises a single lysine in its amino acid sequence, wherein the single lysine is located at at least one position selected from: 43, 64, 75 and 83 (according to Kabat). As shown in the examples, the inventors have surprisingly found that performing point mutations at positions 43, 64 and/or 75 (according to Kabat) does not compromise the Tm and affinity of the resulting ISVDs. Preferably, the at least one ISVD comprises a single lysine at two positions selected from: 43, 64, 75 and 83 (according to Kabat) and comprises an amino acid selected from glutamine, histidine, arginine, alanine, and glutamate, preferably arginine, at the other position of 43, 64 and 75. Even more preferably, the at least one ISVD comprises a single lysine at two positions selected from: 43, 64, 75 and 83 (according to Kabat) and comprises an amino acid selected from glutamine, histidine, arginine, and glutamate, preferably arginine, at the other position of 43, 64 and 75. Preferably, the at least one ISVD comprises a single lysine at two positions selected from: 43, 64, 75 and 83 (according to Kabat) and comprises an amino acid selected from glutamine, histidine, arginine, alanine, and glutamate, preferably arginine, at the other position of 43, 64, 75 and 83. Even more preferably, the at least one ISVD comprises a single lysine at two positions selected from: 43, 64, 75 and 83 (according to Kabat) and comprises an amino acid selected from glutamine, histidine, arginine, and glutamate, preferably arginine, at the other position of 43, 64, 75 and 83.

In another embodiment, the at least one ISVD comprised in the molecule of the present technology comprises two lysines in its amino acid sequence, wherein the two lysine are located at at least two positions selected from: 43, 64, 75 and 83 (according to Kabat). As shown in the examples, the inventors have surprisingly found that performing point mutations at positions 43, 64 and/or 75 (according to Kabat) does not compromise the Tm and affinity of the resulting ISVDs. Preferably, the at least one ISVD comprises two lysines at two positions selected from: 43, 64, 75 and 83 (according to Kabat) and comprises an amino acid selected from glutamine, histidine, arginine, alanine, and glutamate, preferably arginine, at the other position of 43, 64 and 75. Even more preferably, the at least one ISVD comprises two lysines at two positions selected from: 43, 64, 75 and 83 (according to Kabat) and comprises an amino acid selected from glutamine, histidine, arginine, and glutamate, preferably arginine, at the other position of 43, 64 and 75. Preferably, the at least one ISVD comprises two lysines at two positions selected from: 43, 64, 75 and 83 (according to Kabat) and comprises an amino acid selected from glutamine, histidine, arginine, alanine, and glutamate, preferably arginine, at the other position of 43, 64, 75 and 83. Even more preferably, the at least one ISVD comprises two lysines at two positions selected from: 43, 64, 75 and 83 (according to Kabat) and comprises an amino acid selected from glutamine, histidine, arginine, and glutamate, preferably arginine, at the other position of 43, 64, 75 and 83.

In one embodiment, the ISVD of the present technology comprises only two lysines in its amino acid sequence, wherein the two lysines are located at positions 43, 64, 75 and/or 83 (according to Kabat). In another embodiment, the ISVD of the present technology comprises two lysines, wherein the two lysines are located at positions 43, 64, 75 and/or 83 (according to Kabat).

In another embodiment, the at least one ISVD comprised in the molecule of the present technology does not comprise any lysine in its sequence. Hence, in this embodiment, the at least one ISVD either does not comprise any attaching point which is a primary amine or comprises a N-terminal primary amine. As shown in the examples, the inventors have surprisingly found that the lack of lysines in the sequence of ISVDs do not compromise the Tm and affinity of the resulting ISVDs. Preferably, the at least one ISVD does not comprise any lysine in its sequence and comprises an amino acid selected from glutamine, histidine, arginine, alanine, and glutamate, preferably arginine, at positions 43, 64 and/or 75. Preferably, the at least one ISVD does not comprise any lysine in its sequence and comprises an amino acid selected from glutamine, histidine, arginine, alanine, and glutamate, preferably arginine, at positions 43, 64, 75 and/or 83. Preferably, the at least one ISVD does not comprise any lysine in its sequence and comprises an amino acid selected from glutamine, histidine, arginine, alanine, and glutamate, preferably arginine, at all of positions 43, 64 and 75. Even more preferably, the at least one ISVD does not comprise any lysine in its sequence and comprises an amino acid selected from glutamine, histidine, arginine, and glutamate, preferably arginine, at all of positions 43, 64 and 75. Preferably, the at least one ISVD does not comprise any lysine in its sequence and comprises an amino acid selected from glutamine, histidine, arginine, alanine, and glutamate, preferably arginine, at all of positions 43, 64, 75 and 83. Even more preferably, the at least one ISVD does not comprise any lysine in its sequence and comprises an amino acid selected from glutamine, histidine, arginine, and glutamate, preferably arginine, at all of positions 43, 64, 75 and 83.

In one embodiment, the ISVD of the present technology does not comprise any primary amine in its sequence, i.e., it does not comprise any lysine in its sequence, and it also does not comprise a N-terminal primary amine (e.g., because the N-terminal comprises a pyroglutamate modification).

Site-specific conjugation on attachment points or conjugation sites

“Site-specific” conjugation of ISVD-cargo conjugates, in the context of the present technology, refers to the process of attaching cargos (as defined herein, such as half-life extending moieties, targeting moieties, therapeutic moieties or precursors therefrom, imaging moieties, toxic molecules, drugs, nucleic acids, vitamins, etc.) to specific sites or positions in the ISVD. This process ensures that the cargo attaches to the designated location on the ISVD, thereby minimizing the chance of random or nonspecific binding. By conjugating a cargo to a specific site on the antigen-binding molecule, such as an antibody or an ISVD, site-specific conjugation helps maintain the binding affinity and specificity of the antigen-binding molecule for its target antigen, while also optimizing the pharmacokinetics and therapeutic efficacy of the drug. It enables precise control of the drug-to-antibody ratio (DAR), ensuring consistent and well-defined drug loading on each antibody molecule (see, e.g., Pharm Res (2015) 32:3480-3493). Hence, site-specific conjugation requires the designed positioning of attachment points or conjugations sites in the ISVD sequence, so that the conjugation with cargos (as defined herein) takes place at the desired position(s) and with the desired ISVD-drug ratio. The at least one ISVD present in the molecule of the present technology is thus suitable for site specific conjugation of cargos to the one or more conjugation sites or attachment points comprised in the ISVD, if any, wherein the conjugation sites or attachment points are primary amines (e.g., present in the side chain of a Lys or at the N-terminus). Hence, the present technology provides an ISVD which can be used for site-specific conjugation on one or more primary amines present in the side chain of a natural amino acid, lysine, and/or at the N-terminal end of the ISVD. Hence, the present technology provides an ISVD that comprises one or more primary amines present in the side chain of a natural amino acid, lysine, and/or at the N-terminal end of the ISVD and that can be used for site-specific conjugation.

Molecules comprising at least one ISVD and at least one cargo

In one embodiment, at least one of the attachment points or conjugation sites present in the ISVD is linked (directly or via a linker) to a cargo, as defined herein. In a preferred embodiment, the molecule of the present technology comprises at least one ISVD and at least one cargo, wherein the at least one cargo is attached or conjugated to the at least one ISVD through the at least one attachment point or conjugation site. A “cargo” may be any molecule which is/may be attached or conjugated to the ISVD through the attachment point(s) or conjugation site(s) present therein. For instance, cargos which may be attached or conjugated to the ISVD of the present technology are proteins, peptides, ISVDs (such as V_{HH}, V_{L} or V_{H}), polyethylene glycol (PEG), small molecules, glycans, lipids, chelators, fluorophores, radio isotopes, vitamins, nucleic acids, etc. The cargo may have different functionalities. For instance, the at least one cargo may be a half-life extending (HLE) molecule, a targeting molecule, a therapeutic molecule or precursor thereof, an imaging molecule, a toxic molecule, an agonist, a T-cell engagement molecule, a sweeping/degrader molecule, a cell-penetrating molecule, a nuclear localization molecule, a blood brain barrier (BBB) shuttle, a radiotherapeutic molecule or an imaging probe.

Hence, in another embodiment, the molecule of the present technology comprises at least one ISVD and at least one cargo, wherein the cargo is attached or conjugated to the at least one ISVD through the at least one attachment point or conjugation site, and wherein the cargo is a HLE molecule, such as an albumin-binding ISVD (as described herein) or a PEG molecule, or ELNN polypeptides, as described herein. In another embodiment, the molecule of the present technology comprises at least one ISVD and at least one cargo, wherein the cargo is attached or conjugated to the at least one ISVD through the at least one attachment point or conjugation site, and wherein the cargo is a targeting moiety and/or a therapeutic moiety as described herein. In a further embodiment, the molecule of the present technology comprises at least one ISVD and at least two cargos, wherein the cargos are attached or conjugated to the at least one ISVD through at least two attachment points or conjugation sites, wherein the at least two cargos are one HLE molecule, as described herein, and one therapeutic and/or targeting moiety, as described herein.

Hence, the at least one conjugation site present in the at least one ISVD comprised in the molecule of the present technology allows for conjugation of different cargos (directly or by means of a linker, as it will be clear to the skilled person and described in detail below). The skilled person is aware of ways of attaching cargos to the conjugation site(s) present in the ISVD. For instance, Spicer C. D. et al. (“Achieving controlled biomolecule-biomaterial conjugation”, Chem Rev. 2018, 118 (16): 7702-7743), the content of which is herewith incorporated by reference, provides a review on the chemistry of biomolecule conjugation and provide a comprehensive overview of the key strategies for achieving controlled functionalization.

For instance, if the conjugation site is the N-terminal primary amine of the ISVD and/or the primary amine present in the side chain of a Lys preferably located at a solvent-accessible position in the ISVD, the cargo may be attached or conjugated to the ISVD (directly or by means of a linker) by reaction of a group present in the cargo/linker (e.g., isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, or fluorophenyl esters) and the primary amine. See, e.g., Bioconjugate Techniques (Third edition), 2013, Chapter 3-“The reactions of bioconjugation”, Greg T.Hermanson.

### Examples of ISVDs

In one embodiment, the ISVD of the present technology comprises or consists of three complementarity determining regions (CDR1 to CDR3, respectively), wherein:
CDR1 comprises the amino acid sequence of SEQ ID NO: 79 (GLTFSTYTMG) (or has 3, 2 or 1 amino acid difference(s) with SEQ ID NO: 79);
CDR2 comprises the amino acid sequence of SEQ ID NO: 80 (AIIWSGSNTY) (or has 3, 2 or 1 amino acid difference(s) with SEQ ID NO: 80); and
CDR3 comprises the amino acid sequence of SEQ ID NO: 81 (QHFGPIGLTTRGYHY) (or has 3, 2 or 1 amino acid difference(s) with SEQ ID NO: 81);

wherein the CDR sequences are determined according to AbM definition.

In one embodiment, the ISVD of the present technology comprises or consists of three complementarity determining regions (CDR1 to CDR3, respectively), wherein:
CDR1 comprises the amino acid sequence of SEQ ID NO: 264 (TYTMG) (or has 3, 2 or 1
CDR2 comprises the amino acid sequence of SEQ ID NO: 265 (AIIWSGSNTYYADSVHG) (or has 3, 2 or 1 amino acid difference(s) with SEQ ID NO: 265); and CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.

In another embodiment, the ISVD of the present technology comprises or consists of three complementarity determining regions (CDR1 to CDR3, respectively), wherein:
CDR1 comprises the amino acid sequence of SEQ ID NO: 264 (TYTMG) (or has 3, 2 or 1 amino acid difference(s) with SEQ ID NO: 264);
CDR2 comprises the amino acid sequence of SEQ ID NO: 304 (AIIWSGSNTYYADSVKG) (or has 3, 2 or 1 amino acid difference(s) with SEQ ID NO: 304); and
CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.

In another embodiment, the ISVD of the present technology comprises or consists of three complementarity determining regions (CDR1 to CDR3, respectively), wherein:
CDR1 comprises the amino acid sequence of SEQ ID NO: 264 (TYTMG) (or has 3, 2 or 1 amino acid difference(s) with SEQ ID NO: 264);
CDR2 comprises the amino acid sequence of SEQ ID NO: 281 (AIIWSGSNTYYADSVRG) (or has 3, 2 or 1 amino acid difference(s) with SEQ ID NO: 281); and
CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.

In another embodiment, the ISVD of the present technology comprises or consists of three complementarity determining regions (CDR1 to CDR3, respectively), wherein:
CDR1 comprises the amino acid sequence of SEQ ID NO: 264 (TYTMG) (or has 3, 2 or 1
CDR2 comprises the amino acid sequence of SEQ ID NO: 280 (AIIWSGSNTYYADSVEG) (or has 3, 2 or 1 amino acid difference(s) with SEQ ID NO: 280); and
CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.

In another embodiment, the ISVD of the present technology comprises or consists of three complementarity determining regions (CDR1 to CDR3, respectively), wherein:
CDR1 comprises the amino acid sequence of SEQ ID NO: 264 (TYTMG) (or has 3, 2 or 1 amino acid difference(s) with SEQ ID NO: 264);
CDR2 comprises the amino acid sequence of SEQ ID NO: 282 (AIIWSGSNTYYADSVQG) (or has 3, 2 or 1 amino acid difference(s) with SEQ ID NO: 282); and
CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.

In another embodiment, the ISVD of the present technology comprises or consists of three complementarity determining regions (CDR1 to CDR3, respectively), wherein:
CDR1 comprises the amino acid sequence of SEQ ID NO: 264 (TYTMG) (or has 3, 2 or 1 amino acid difference(s) with SEQ ID NO: 264);
CDR2 comprises the amino acid sequence of SEQ ID NO: 283 (AIIWSGSNTYYADSVAG) (or has 3, 2 or 1 amino acid difference(s) with SEQ ID NO: 283); and
CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.

For instance, the ISVD of the present technology may be selected from an ISVD comprising or consisting of SEQ ID NO.: 3-25, see Table 4, or a sequence which has at least 80%, such as at least 85%, or at least 90% or at least 95%, or at least 99% identity with a sequence as defined in SEQ IS NO.: 3-25.

TABLE 4
Sequence of CEACAM5-binding ISVDs derived from A0315007E07
(positions in the description are numbered according to Kabat) -
A031500384/T028501789 is the precursor

| | SEQ | | |
|---|---|---|---|
| | ID | | Description (Mutations |
| ID | NO | Amino acid sequence | related to invention) |
| T028501790 | 3 | EVQLVESGGGVVQPGGSLRLSCAASGLTFS | A0315007E07(K43E) |
| | | TYTMGWFRQAPGEEREFVAAIIWSGSNTY | |
| | | YADSVKGRFTISRDNAKNTVYLQMNSLRPE | |
| | | DTALYYCAAQHFGPIGLTTRGYHYWGQGT | |
| | | LVTVSS | |
| T028501791 | 4 | EVQLVESGGGVVQPGGSLRLSCAASGLTFS | A0315007E07(K43Q) |
| | | TYTMGWFRQAPGQEREFVAAIIWSGSNTY | |
| | | YADSVKGRFTISRDNAKNTVYLQMNSLRPE | |
| | | DTALYYCAAQHFGPIGLTTRGYHYWGQGT | |
| | | LVTVSS | |
| T028501792 | 5 | EVQLVESGGGVVQPGGSLRLSCAASGLTFS | A0315007E07(K43R) |
| | | TYTMGWFRQAPGREREFVAAIIWSGSNTY | |
| | | YADSVKGRFTISRDNAKNTVYLQMNSLRPE | |
| | | DTALYYCAAQHFGPIGLTTRGYHYWGQGT | |
| | | LVTVSS | |
| T028501793 | 6 | EVQLVESGGGVVQPGGSLRLSCAASGLTFS | A0315007E07(K43H) |
| | | TYTMGWFRQAPGHEREFVAAIIWSGSNTY | |
| | | YADSVKGRFTISRDNAKNTVYLQMNSLRPE | |
| | | DTALYYCAAQHFGPIGLTTRGYHYWGQGT | |
| | | LVTVSS | |
| T028501794 | 7 | EVQLVESGGGVVQPGGSLRLSCAASGLTFS | A0315007E07(K43A) |
| | | TYTMGWFRQAPGAEREFVAAIIWSGSNTY | |
| | | YADSVKGRFTISRDNAKNTVYLQMNSLRPE | |
| | | DTALYYCAAQHFGPIGLTTRGYHYWGQGT | |
| | | LVTVSS | |
| T028501795 | 8 | EVQLVESGGGVVQPGGSLRLSCAASGLTFS | A0315007E07(K64E) |
| | | TYTMGWFRQAPGKEREFVAAIIWSGSNTY | |
| | | YADSVEGRFTISRDNAKNTVYLQMNSLRPE | |
| | | DTALYYCAAQHFGPIGLTTRGYHYWGQGT | |
| | | LVTVSS | |
| T028501796 | 9 | EVQLVESGGGVVQPGGSLRLSCAASGLTFS | A0315007E07(K64Q) |
| | | TYTMGWFRQAPGKEREFVAAIIWSGSNTY | |
| | | YADSVQGRFTISRDNAKNTVYLQMNSLRPE | |
| | | DTALYYCAAQHFGPIGLTTRGYHYWGQGT | |
| | | LVTVSS | |
| T028501797 | 10 | EVQLVESGGGVVQPGGSLRLSCAASGLTFS | A0315007E07(K64R) |
| | | TYTMGWFRQAPGKEREFVAAIIWSGSNTY | |
| | | YADSVRGRFTISRDNAKNTVYLQMNSLRPE | |
| | | DTALYYCAAQHFGPIGLTTRGYHYWGQGT | |
| | | LVTVSS | |
| T028501798 | 11 | EVQLVESGGGVVQPGGSLRLSCAASGLTFS | A0315007E07(K64H) |
| | | TYTMGWFRQAPGKEREFVAAIIWSGSNTY | |
| | | YADSVHGRFTISRDNAKNTVYLQMNSLRPE | |
| | | DTALYYCAAQHFGPIGLTTRGYHYWGQGT | |
| | | LVTVSS | |
| T028501799 | 12 | EVQLVESGGGVVQPGGSLRLSCAASGLTFS | A0315007E07(K64A) |
| | | TYTMGWFRQAPGKEREFVAAIIWSGSNTY | |
| | | YADSVAGRFTISRDNAKNTVYLQMNSLRPE | |
| | | DTALYYCAAQHFGPIGLTTRGYHYWGQGT | |
| | | LVTVSS | |
| T028501800 | 13 | EVQLVESGGGVVQPGGSLRLSCAASGLTFS | A0315007E07(K75E) |
| | | TYTMGWFRQAPGKEREFVAAIIWSGSNTY | |
| | | YADSVKGRFTISRDNAENTVYLQMNSLRPE | |
| | | DTALYYCAAQHFGPIGLTTRGYHYWGQGT | |
| | | LVTVSS | |
| T028501801 | 14 | EVQLVESGGGVVQPGGSLRLSCAASGLTFS | A0315007E07(K75Q) |
| | | TYTMGWFRQAPGKEREFVAAIIWSGSNTY | |
| | | YADSVKGRFTISRDNAQNTVYLQMNSLRPE | |
| | | DTALYYCAAQHFGPIGLTTRGYHYWGQGT | |
| | | LVTVSS | |
| T028501802 | 15 | EVQLVESGGGVVQPGGSLRLSCAASGLTFS | A0315007E07(K75R) |
| | | TYTMGWFRQAPGKEREFVAAIIWSGSNTY | |
| | | YADSVKGRFTISRDNARNTVYLQMNSLRPE | |
| | | DTALYYCAAQHFGPIGLTTRGYHYWGQGT | |
| | | LVTVSS | |
| T028501803 | 16 | EVQLVESGGGVVQPGGSLRLSCAASGLTFS | A0315007E07(K75H) |
| | | TYTMGWFRQAPGKEREFVAAIIWSGSNTY | |
| | | YADSVKGRFTISRDNAHNTVYLQMNSLRPE | |
| | | DTALYYCAAQHFGPIGLTTRGYHYWGQGT | |
| | | LVTVSS | |
| T028501804 | 17 | EVQLVESGGGVVQPGGSLRLSCAASGLTFS | A0315007E07(K75A) |
| | | TYTMGWFRQAPGKEREFVAAIIWSGSNTY | |
| | | YADSVKGRFTISRDNAANTVYLQMNSLRPE | |
| | | DTALYYCAAQHFGPIGLTTRGYHYWGQGT | |
| | | LVTVSS | |
| T039400047 | 18 | EVQLVESGGGVVQPGGSLRLSCAASGLTFS | HIS6-ENLYFQS-5GS- |
| | | TYTMGWFRQAPGREREFVAAIIWSGSNTY | A0315007E07(K43R, |
| | | YADSVKGRFTISRDNARNTVYLQMNSLRPE | K75R) |
| | | DTALYYCAAQHFGPIGLTTRGYHYWGQGT | |
| | | LVTVSS | |
| T039400048 | 19 | EVQLVESGGGVVQPGGSLRLSCAASGLTFS | HIS6-ENLYFQS-5GS- |
| | | TYTMGWFRQAPGREKEFVAAIIWSGSNTY | A0315007E07(K43R, |
| | | YADSVRGRFTISRDNARNTVYLQMNSLRPE | R45K, K64R, K75R) |
| | | DTALYYCAAQHFGPIGLTTRGYHYWGQGT | |
| | | LVTVSS | |
| T039400049 | 20 | EVQLVESGGGVVQPGGSLRLSCAASGLTFS | HIS6-ENLYFQS-5GS- |
| | | TYTMGWFRQAPGREREFVAAIIWSGSNTY | A0315007E07(K43R, |
| | | YADSVRGRFTISRDNARNTVYLQMNSLRPE | K64R, K75R, Q105K) |
| | | DTALYYCAAQHFGPIGLTTRGYHYWGKGTL | |
| | | VTVSS | |
| T039400050 | 21 | EVQLVESGGGVVQPGGSLRLSCAASGLTFS | HIS6-ENLYFQS-5GS- |
| | | TYTMGWFRQAPGREREFVAAIIWSGSNTY | A0315007E07(K43R, |
| | | YADSVRGRFTISRDNARNTVYLQMNSLRPE | K64R, K75R) |
| | | DTALYYCAAQHFGPIGLTTRGYHYWGQGT | |
| | | LVTVSS | |
| T039400051 | 22 | EVQLVESGGGVVQPGGSLRLSCAASGLTFS | HIS6-ENLYFQS-5GS- |
| | | TYTMGWFRQAPGREREFVAAIIWSGSNTY | A0315007E07(K43R, |
| | | YADSVRGRFTISRDNAKNTVYLQMNSLRPE | K64R,) |
| | | DTALYYCAAQHFGPIGLTTRGYHYWGQGT | |
| | | LVTVSS | |
| T039400052 | 23 | EVQLVESGGGVVQPGGSLRLSCAASGLTFS | HIS6-ENLYFQS-5GS- |
| | | TYTMGWFRQAPGREREFVAAIIWSGSNTY | A0315007E07K43R, |
| | | YADSVRGRFTISRDKARNTVYLQMNSLRPE | K64R, N73K, K75R) |
| | | DTALYYCAAQHFGPIGLTTRGYHYWGQGT | |
| | | LVTVSS | |
| T039400053 | 24 | EVQLVESGGGVVQPGGSLRLSCAASGLTFS | HIS6-ENLYFQS-5GS- |
| | | TYTMGWFRQAPGKEREFVAAIIWSGSNTY | A0315007E07(K64R, |
| | | YADSVRGRFTISRDNARNTVYLQMNSLRPE | K75R,) |
| | | DTALYYCAAQHFGPIGLTTRGYHYWGQGT | |
| | | LVTVSS | |
| T039400054 | 25 | EVQLVESGGGVVQPGGSLRLSCAASGLTFS | HIS6-ENLYFQS-5GS- |
| | | TYTMGWFRQAPGREREFVAAIIWSGSNTY | A0315007E07(K43R, |
| | | YADSVRGRFTISRDNARNTVYLQMNSLRPE | K64R, K75R,)-K |
| | | DTALYYCAAQHFGPIGLTTRGYHYWGQGT | |
| | | LVTVSSK | |

In another embodiment, the ISVD of the present technology comprises three complementarity determining regions (CDR1 to CDR3, respectively), wherein:

CDR1 comprises the amino acid sequence of SEQ ID NO: 82 (GHTFSEYALG) (or has 3, 2 or 1 amino acid difference(s) with SEQ ID NO: 82);
CDR2 comprises the amino acid sequence of SEQ ID NO: 83 (AlNWGGGWTY) (or has 3, 2 or 1 amino acid difference(s) with SEQ ID NO: 83); and
CDR3 comprises the amino acid sequence of SEQ ID NO: 84 (SSDYAGGNPTGYPY) (or has 3, 2 or 1 amino acid difference(s) with SEQ ID NO: 84);

wherein the CDR sequences are determined according to AbM definition.

In one embodiment, the ISVD of the present technology comprises or consists of three complementarity determining regions (CDR1 to CDR3, respectively), wherein:
CDR1 comprises the amino acid sequence of SEQ ID NO: 266 (EYALG) (or has 3, 2 or 1 amino acid difference(s) with SEQ ID NO: 266);
CDR2 comprises the amino acid sequence of SEQ ID NO: 267 (AlNWGGGWTYYADSVKG) (or has 3, 2 or 1 amino acid difference(s) with SEQ ID NO: 267); and
CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.

In one embodiment, the ISVD of the present technology comprises or consists of three complementarity determining regions (CDR1 to CDR3, respectively), wherein:
CDR1 comprises the amino acid sequence of SEQ ID NO: 266 (EYALG) (or has 3, 2 or 1 amino acid difference(s) with SEQ ID NO: 266);
CDR2 comprises the amino acid sequence of SEQ ID NO: 284 (AlNWGGGWTYYADSVEG) (or has 3, 2 or 1 amino acid difference(s) with SEQ ID NO: 284); and
CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.

In one embodiment, the ISVD of the present technology comprises or consists of three complementarity determining regions (CDR1 to CDR3, respectively), wherein:
CDR1 comprises the amino acid sequence of SEQ ID NO: 266 (EYALG) (or has 3, 2 or 1 amino acid difference(s) with SEQ ID NO: 266);
CDR2 comprises the amino acid sequence of SEQ ID NO: 285 (AlNWGGGWTYYADSVQG) (or has 3, 2 or 1 amino acid difference(s) with SEQ ID NO: 285); and
CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.

In one embodiment, the ISVD of the present technology comprises or consists of three complementarity determining regions (CDR1 to CDR3, respectively), wherein:
CDR1 comprises the amino acid sequence of SEQ ID NO: 266 (EYALG) (or has 3, 2 or 1 amino acid difference(s) with SEQ ID NO: 266);
CDR2 comprises the amino acid sequence of SEQ ID NO: 286 (AlNWGGGWTYYADSVRG) (or has 3, 2 or 1 amino acid difference(s) with SEQ ID NO: 286); and
CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.

In one embodiment, the ISVD of the present technology comprises or consists of three complementarity determining regions (CDR1 to CDR3, respectively), wherein:
CDR1 comprises the amino acid sequence of SEQ ID NO: 266 (EYALG) (or has 3, 2 or 1 amino acid difference(s) with SEQ ID NO: 266);
CDR2 comprises the amino acid sequence of SEQ ID NO: 287 (AlNWGGGWTYYADSVHG) (or has 3, 2 or 1 amino acid difference(s) with SEQ ID NO: 287); and
CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.

In one embodiment, the ISVD of the present technology comprises or consists of three complementarity determining regions (CDR1 to CDR3, respectively), wherein:
CDR1 comprises the amino acid sequence of SEQ ID NO: 266 (EYALG) (or has 3, 2 or 1 amino acid difference(s) with SEQ ID NO: 266);
CDR2 comprises the amino acid sequence of SEQ ID NO: 288 (AlNWGGGWTYYADSVAG) (or has 3, 2 or 1 amino acid difference(s) with SEQ ID NO: 288); and
CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.

Preferably, the at least one ISVD may be selected from an ISVD comprising or consisting of SEQ ID NO.: 28-42, see Table 5, or a sequence which has at least 80%, such as at least 85%, or at least 90% or at least 95%, or at least 99% identity with a sequence as defined in SEQ IS NO.: 28-42.

TABLE 5
Sequence of CEACAM5-binding ISVDs derived from
A0315024B02 (positions in the description are
numbered according to Kabat) - A031500099/
T028501817 is the precursor

| | | | Description |
|---|---|---|---|
| | SEQ | | (Mutations |
| | ID | | related to |
| ID | NO | Amino acid sequence | invention) |
| T028501818 | 28 | EVQLVESGGGVVQPGGSLRL | A0315024B02 |
| | | SCAASGHTFSEYALGWFRQA | (M43E, |
| | | PGEEREFVAAINWGGGWTYY | Q64K, K83R) |
| | | ADSVKGRFTISRDNAKNTLY | |
| | | LQMNSLRPEDTALYYCAASS | |
| | | DYAGGNPTGYPYWGQGTLVT | |
| | | VSS | |
| T028501819 | 29 | EVQLVESGGGVVQPGGSLRL | A0315024B02 |
| | | SCAASGHTFSEYALGWFRQA | (M43Q, |
| | | PGQEREFVAAINWGGGWTYY | Q64K, K83R) |
| | | ADSVKGRFTISRDNAKNTLY | |
| | | LQMNSLRPEDTALYYCAASS | |
| | | DYAGGNPTGYPYWGQGTLVT | |
| | | VSS | |
| T028501820 | 30 | EVQLVESGGGVVQPGGSLRL | A0315024B02 |
| | | SCAASGHTFSEYALGWFRQA | (M43R, |
| | | PGREREFVAAINWGGGWTYY | Q64K, K83R) |
| | | ADSVKGRFTISRDNAKNTLY | |
| | | LQMNSLRPEDTALYYCAASS | |
| | | DYAGGNPTGYPYWGQGTLVT | |
| | | VSS | |
| T028501821 | 31 | EVQLVESGGGVVQPGGSLRL | A0315024B02 |
| | | SCAASGHTFSEYALGWFRQA | (M43H, |
| | | PGHEREFVAAINWGGGWTYY | Q64K, K83R) |
| | | ADSVKGRFTISRDNAKNTLY | |
| | | LQMNSLRPEDTALYYCAASS | |
| | | DYAGGNPTGYPYWGQGTLVT | |
| | | VSS | |
| T028501822 | 32 | EVQLVESGGGVVQPGGSLRL | A0315024B02 |
| | | SCAASGHTFSEYALGWFRQA | (M43A, |
| | | PGAEREFVAAINWGGGWTYY | Q64K, K83R) |
| | | ADSVKGRFTISRDNAKNTLY | |
| | | LQMNSLRPEDTALYYCAASS | |
| | | DYAGGNPTGYPYWGQGTLVT | |
| | | VSS | |
| T028501823 | 33 | EVQLVESGGGVVQPGGSLRL | A0315024B02 |
| | | SCAASGHTFSEYALGWFRQA | (M43K, |
| | | PGKEREFVAAINWGGGWTYY | Q64E, K83R) |
| | | ADSVEGRFTISRDNAKNTLY | |
| | | LQMNSLRPEDTALYYCAASS | |
| | | DYAGGNPTGYPYWGQGTLVT | |
| | | VSS | |
| T028501824 | 34 | EVQLVESGGGVVQPGGSLRL | A0315024B02 |
| | | SCAASGHTFSEYALGWFRQA | (M43K, |
| | | PGKEREFVAAINWGGGWTYY | (K64K83R) |
| | | ADSVQGRFTISRDNAKNTLY | |
| | | LQMNSLRPEDTALYYCAASS | |
| | | DYAGGNPTGYPYWGQGTLVT | |
| | | VSS | |
| T028501825 | 35 | EVQLVESGGGVVQPGGSLRL | A0315024B02 |
| | | SCAASGHTFSEYALGWFRQA | (M43K, |
| | | PGKEREFVAAINWGGGWTYY | Q64R, K83R) |
| | | ADSVRGRFTISRDNAKNTLY | |
| | | LQMNSLRPEDTALYYCAASS | |
| | | DYAGGNPTGYPYWGQGTLVT | |
| | | VSS | |
| T028501826 | 36 | EVQLVESGGGVVQPGGSLRL | A0315024B02 |
| | | SCAASGHTFSEYALGWFRQA | (M43K, |
| | | PGKEREFVAAINWGGGWTYY | Q64H, K83R) |
| | | ADSVHGRFTISRDNAKNTLY | |
| | | LQMNSLRPEDTALYYCAASS | |
| | | DYAGGNPTGYPYWGQGTLVT | |
| | | VSS | |
| T028501827 | 37 | EVQLVESGGGVVQPGGSLRL | A0315024B02 |
| | | SCAASGHTFSEYALGWFRQA | (M43K, |
| | | PGKEREFVAAINWGGGWTYY | Q64A, K83R) |
| | | ADSVAGRFTISRDNAKNTLY | |
| | | LQMNSLRPEDTALYYCAASS | |
| | | DYAGGNPTGYPYWGQGTLVT | |
| | | VSS | |
| T028501828 | 38 | EVQLVESGGGVVQPGGSLRL | A0315024B02 |
| | | SCAASGHTFSEYALGWFRQA | (M43K, |
| | | PGKEREFVAAINWGGGWTYY | Q64K, K75E, |
| | | ADSVKGRFTISRDNAENTLY | K83R) |
| | | LQMNSLRPEDTALYYCAASS | |
| | | DYAGGNPTGYPYWGQGTLVT | |
| | | VSS | |
| T028501829 | 39 | EVQLVESGGGVVQPGGSLRL | A0315024B02 |
| | | SCAASGHTFSEYALGWFRQA | (M43K, |
| | | PGKEREFVAAINWGGGWTYY | Q64K, K75Q, |
| | | ADSVKGRFTISRDNAQNTLY | K83R) |
| | | LQMNSLRPEDTALYYCAASS | |
| | | DYAGGNPTGYPYWGQGTLVT | |
| | | VSS | |
| T028501830 | 40 | EVQLVESGGGVVQPGGSLRL | A0315024B02 |
| | | SCAASGHTFSEYALGWFRQA | (M43K, |
| | | PGKEREFVAAINWGGGWTYY | Q64K, K75R, |
| | | ADSVKGRFTISRDNARNTLY | K83R) |
| | | LQMNSLRPEDTALYYCAASS | |
| | | DYAGGNPTGYPYWGQGTLVT | |
| | | VSS | |
| T028501831 | 41 | EVQLVESGGGVVQPGGSLRL | A0315024B02 |
| | | SCAASGHTFSEYALGWFRQA | (M43K, |
| | | PGKEREFVAAINWGGGWTYY | Q64K, K75H, |
| | | ADSVKGRFTISRDNAHNTLY | K83R) |
| | | LQMNSLRPEDTALYYCAASS | |
| | | DYAGGNPTGYPYWGQGTLVT | |
| | | VSS | |
| T028501832 | 42 | EVQLVESGGGVVQPGGSLRL | A0315024B02 |
| | | SCAASGHTFSEYALGWFRQA | (M43K, |
| | | PGKEREFVAAINWGGGWTYY | Q64K,K75A, |
| | | ADSVKGRFTISRDNAANTLY | K83R) |
| | | LQMNSLRPEDTALYYCAASS | |
| | | DYAGGNPTGYPYWGQGTLVT | |
| | | VSS | |

In another embodiment, the ISVD of the present technology comprises three complementarity determining regions (CDR1 to CDR3, respectively), wherein:
CDR1 comprises the amino acid sequence of SEQ ID NO: 85 (GFTFRSFGMS) (or has 3, 2 or 1 amino acid difference(s) with SEQ ID NO: 85);
CDR2 comprises the amino acid sequence of SEQ ID NO: 86 (SISGSGSDTL) (or has 3, 2 or 1 amino acid difference(s) with SEQ ID NO: 86); and
CDR3 comprises the amino acid sequence of SEQ ID NO: 87 (GGSLSR) (or has 3, 2 or 1 amino acid difference(s) with SEQ ID NO: 87);

wherein the CDR sequences are determined according to AbM definition.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 269 (SISGSGSDTLYADSVKG) (or has 3, 2 or 1 amino acid difference(s) with SEQ ID NO: 269); and
CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 289 (SISGSGSDTLYADSVEG) (or has 3, 2 or 1 amino acid difference(s) with SEQ ID NO: 289); and
CDR3 comprises the amino acid sequence of SEQ ID NO: 87 (GGSLSR) (or has 3, 2 or 1 amino acid difference(s) with SEQ ID NO: 87);

wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 290 (SISGSGSDTLYADSVQG) (or has 3, 2 or 1 amino acid difference(s) with SEQ ID NO: 290); and
CDR3 comprises the amino acid sequence of SEQ ID NO: 87 (GGSLSR) (or has 3, 2 or 1 amino acid difference(s) with SEQ ID NO: 87);

wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 291 (SISGSGSDTLYADSVHG) (or has 3, 2 or 1 amino acid difference(s) with SEQ ID NO: 291); and
CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 292 (SISGSGSDTLYADSVRG) (or has 3, 2 or 1 amino acid difference(s) with SEQ ID NO: 292); and
CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 293 (SISGSGSDTLYADSVAG) (or has 3, 2 or 1 amino acid difference(s) with SEQ ID NO: 293); and
CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 268 (SFGMS) (or has 3, 2 or 1 amino acid difference(s) with SEQ ID NO: 268);
CDR2 comprises the amino acid sequence of SEQ ID NO: 295 (SISGSGSDTLYAKSVRG) (or has 3, 2 or 1 amino acid difference(s) with SEQ ID NO: 295); and
CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.

Preferably, the at least one ISVD may be selected from an ISVD comprising or consisting of SEQ ID NO.: 45-67, 228-249, see Table 6, or a sequence which has at least 80%, such as at least 85%, or at least 90% or at least 95%, or at least 99% identity with a sequence as defined in SEQ IS NO.: 45-67, 228-249.

TABLE 6
Sequence of Human serum albumin-binding ISVDs derived from HSA006A06
(positions in the description are numbered according to Kabat) -
Precursor is T028501805/ALB00622

| | SEQ | | |
|---|---|---|---|
| | ID | | Description (Mutations |
| ID | NO | Amino acid sequence | related to invention) |
| T028501806 | 45 | EVQLVESGGGVVQPGGSLRLSCAASGF | HSA006A06(K43E, K83R) |
| | | TFRSFGMSWVRQAPGEGPEWVSSISGS | |
| | | GSDTLYADSVKGRFTISRDNSKNTLYLQ | |
| | | MNSLRPEDTALYYCTIGGSLSRSSQGTLV | |
| | | TVSS | |
| T028501807 | 46 | EVQLVESGGGVVQPGGSLRLSCAASGF | HSA006A06(K43Q, K83R) |
| | | TFRSFGMSWVRQAPGQGPEWVSSISG | |
| | | SGSDTLYADSVKGRFTISRDNSKNTLYLQ | |
| | | MNSLRPEDTALYYCTIGGSLSRSSQGTLV | |
| | | TVSS | |
| T028501808 | 47 | EVQLVESGGGVVQPGGSLRLSCAASGF | HSA006A06(K43H, K83R) |
| | | TFRSFGMSWVRQAPGHGPEWVSSISG | |
| | | SGSDTLYADSVKGRFTISRDNSKNTLYLQ | |
| | | MNSLRPEDTALYYCTIGGSLSRSSQGTLV | |
| | | TVSS | |
| T028501809 | 48 | EVQLVESGGGVVQPGGSLRLSCAASGF | HSA006A06(K43A, K83R) |
| | | TFRSFGMSWVRQAPGAGPEWVSSISG | |
| | | SGSDTLYADSVKGRFTISRDNSKNTLYLQ | |
| | | MNSLRPEDTALYYCTIGGSLSRSSQGTLV | |
| | | TVSS | |
| T028501810 | 49 | EVQLVESGGGVVQPGGSLRLSCAASGF | HSA006A06(K64E,K83R) |
| | | TFRSFGMSWVRQAPGKGPEWVSSISGS | |
| | | GSDTLYADSVEGRFTISRDNSKNTLYLQ | |
| | | MNSLRPEDTALYYCTIGGSLSRSSQGTLV | |
| | | TVSS | |
| T028501811 | 50 | EVQLVESGGGVVQPGGSLRLSCAASGF | HSA006A06(K64Q, K83R) |
| | | TFRSFGMSWVRQAPGKGPEWVSSISGS | |
| | | GSDTLYADSVQGRFTISRDNSKNTLYLQ | |
| | | MNSLRPEDTALYYCTIGGSLSRSSQGTLV | |
| | | TVSS | |
| T028501812 | 51 | EVQLVESGGGVVQPGGSLRLSCAASGF | HSA006A06(K64H, K83R) |
| | | TFRSFGMSWVRQAPGKGPEWVSSISGS | |
| | | GSDTLYADSVHGRFTISRDNSKNTLYLQ | |
| | | MNSLRPEDTALYYCTIGGSLSRSSQGTLV | |
| | | TVSS | |
| T028501813 | 52 | EVQLVESGGGVVQPGGSLRLSCAASGF | HSA006A06(K75E, K83R) |
| | | TFRSFGMSWVRQAPGKGPEWVSSISGS | |
| | | GSDTLYADSVKGRFTISRDNSENTLYLQ | |
| | | MNSLRPEDTALYYCTIGGSLSRSSQGTLV | |
| | | TVSS | |
| T028501814 | 53 | EVQLVESGGGVVQPGGSLRLSCAASGF | HSA006A06(K75Q, K83R) |
| | | TFRSFGMSWVRQAPGKGPEWVSSISGS | |
| | | GSDTLYADSVKGRFTISRDNSQNTLYLQ | |
| | | MNSLRPEDTALYYCTIGGSLSRSSQGTLV | |
| | | TVSS | |
| T028501815 | 54 | EVQLVESGGGVVQPGGSLRLSCAASGF | HSA006A06(K75R, K83R) |
| | | TFRSFGMSWVRQAPGKGPEWVSSISGS | |
| | | GSDTLYADSVKGRFTISRDNSRNTLYLQ | |
| | | MNSLRPEDTALYYCTIGGSLSRSSQGTLV | |
| | | TVSS | |
| T028501816 | 55 | EVQLVESGGGVVQPGGSLRLSCAASGF | HSA006A06(K75A, K83R) |
| | | TFRSFGMSWVRQAPGKGPEWVSSISGS | |
| | | GSDTLYADSVKGRFTISRDNSANTLYLQ | |
| | | MNSLRPEDTALYYCTIGGSLSRSSQGTLV | |
| | | TVSS | |
| T028501833 | 56 | EVQLVESGGGVVQPGGSLRLSCAASGF | HSA006A06(K43R, K83R) |
| | | TFRSFGMSWVRQAPGRGPEWVSSISGS | |
| | | GSDTLYADSVKGRFTISRDNSKNTLYLQ | |
| | | MNSLRPEDTALYYCTIGGSLSRSSQGTLV | |
| | | TVSS | |
| T028501834 | 57 | EVQLVESGGGVVQPGGSLRLSCAASGF | HSA006A06(K64R, K83R) |
| | | TFRSFGMSWVRQAPGKGPEWVSSISGS | |
| | | GSDTLYADSVRGRFTISRDNSKNTLYLQ | |
| | | MNSLRPEDTALYYCTIGGSLSRSSQGTLV | |
| | | TVSS | |
| T028501835 | 58 | EVQLVESGGGVVQPGGSLRLSCAASGF | HSA006A06(K64A, K83R) |
| | | TFRSFGMSWVRQAPGKGPEWVSSISGS | |
| | | GSDTLYADSVAGRFTISRDNSKNTLYLQ | |
| | | MNSLRPEDTALYYCTIGGSLSRSSQGTLV | |
| | | TVSS | |
| T028501836 | 59 | EVQLVESGGGVVQPGGSLRLSCAASGF | HSA006A06(K83R) |
| | | TFRSFGMSWVRQAPGKGPEWVSSISGS | |
| | | GSDTLYADSVKGRFTISRDNSHNTLYLQ | |
| | | MNSLRPEDTALYYCTIGGSLSRSSQGTLV | |
| | | TVSS | |
| T039400039 | 60 | EVQLVESGGGVVQPGGSLRLSCAASGF | HIS6-ENLYFQS-5GS- |
| | | TFRSFGMSWVRQAPGRGPEWVSSISGS | HSA006A06(K43R, K64R, |
| | | GSDTLYADSVRGRFTISRDNSKNTLYLQ | K83R) |
| | | MNSLRPEDTALYYCTIGGSLSRSSQGTLV | |
| | | TVSS | |
| T039400040 | 61 | EVQLVESGGGVVQPGGSLRLSCAASGF | HIS6-ENLYFQS-5GS- |
| | | TFRSFGMSWVRQAPGRGPEWVSSISGS | HSA006A06(K43R, K64R, |
| | | GSDTLYADSVRGRFTISRDNSRNTLYLQ | K75R, K83R) |
| | | MNSLRPEDTALYYCTIGGSLSRSSQGTLV | |
| | | TVSS | |
| T039400041 | 62 | EVQLVESGGGVVQPGGSLRLSCAASGF | HIS6-ENLYFQS-5GS-HSA006A06 |
| | | TFRSFGMSWVRQAPGRGPEWVSSISGS | (K43R, K75R, K83R) |
| | | GSDTLYADSVKGRFTISRDNSRNTLYLQ | |
| | | MNSLRPEDTALYYCTIGGSLSRSSQGTLV | |
| | | TVSS | |
| T039400042 | 63 | EVQLVESGGGVVQPGGSLRLSCAASGF | HIS6-ENLYFQS-5GS-HSA006A06 |
| | | TFRSFGMSWVRQAPGRGPEWVSSISGS | (K43R, K64R, N73K, K75R, |
| | | GSDTLYADSVRGRFTISRDKSRNTLYLQ | K83R) |
| | | MNSLRPEDTALYYCTIGGSLSRSSQGTLV | |
| | | TVSS | |
| T039400043 | 64 | EVQLVESGGGVVQPGGSLRLSCAASGF | HIS6-ENLYFQS-5GS-HSA006A06 |
| | | TFRSFGMSWVRQAPGKGPEWVSSISGS | (K64R, K75R, K83R) |
| | | GSDTLYADSVRGRFTISRDNSRNTLYLQ | |
| | | MNSLRPEDTALYYCTIGGSLSRSSQGTLV | |
| | | TVSS | |
| T039400044 | 65 | EVQLVESGGGVVQPGGSLRLSCAASGF | HIS6-ENLYFQS-5GS-HSA006A06 |
| | | TFRSFGMSWVRQAPGRGPEWVSSISGS | (K43R, K64R, K75R, K83R, |
| | | GSDTLYADSVRGRFTISRDNSRNTLYLQ | Q105K) |
| | | MNSLRPEDTALYYCTIGGSLSRSSKGTLV | |
| | | TVSS | |
| T039400045 | 66 | EVQLVESGGGVVQPGGSLRLSCAASGF | HIS6-ENLYFQS-5GS-HSA006A06 |
| | | TFRSFGMSWVRQAPGRGKEWVSSISGS | (K43R, P45K,K64R, K75R, |
| | | GSDTLYADSVRGRFTISRDNSRNTLYLQ | K83R) |
| | | MNSLRPEDTALYYCTIGGSLSRSSQGTLV | |
| | | TVSS | |
| T039400046 | 67 | EVQLVESGGGVVQPGGSLRLSCAASGF | HIS6-ENLYFQS-5GS-HSA006A06 |
| | | TFRSFGMSWVRQAPGRGPEWVSSISGS | (K43R, K64R, K75R, K83R)-K |
| | | GSDTLYADSVRGRFTISRDNSRNTLYLQ | |
| | | MNSLRPEDTALYYCTIGGSLSRSSQGTLV | |
| | | TVSSK | |
| ALB00622/T | 43 | EVQLVESGGGVVQPGGSLRLSCAASGF | HSA006A06(K83R) |
| 028501805 | | TFRSFGMSWVRQAPGKGPEWVSSISGS | |
| | | GSDTLYADSVKGRFTISRDNSKNTLYLQ | |
| | | MNSLRPEDTALYYCTIGGSLSRSSQGTLV | |
| | | TVSS | |
| TPP-134274 | 228 | QVQLVESGGGVVQPGGSLRLSCAASGF | HSA006A06(K83R) |
| | | TFRSFGMSWVRQAPGKGPEWVSSISGS | |
| | | GSDTLYADSVKGRFTISRDNSKNTLYLQ | |
| | | MNSLRPEDTALYYCTIGGSLSRSSQGTLV | |
| | | TVSS | |
| TPP-134275 | 229 | QVQLVESGGGVVQPGGSLRLSCAASGF | HSA006A06(K43R, K64R, K75R, |
| | | TFRSFGMSWVRQAPGRGPEWVSSISGS | K83R) |
| | | GSDTLYADSVRGRFTISRDNSRNTLYLQ | |
| | | MNSLRPEDTALYYCTIGGSLSRSSQGTLV | |
| | | TVSS | |
| TPP-134276 | 230 | QVQLVESGGKVVQPGGSLRLSCAASGF | HSA006A06(G10K, K43R, K64R, |
| | | TFRSFGMSWVRQAPGRGPEWVSSISGS | K75R, K83R) |
| | | GSDTLYADSVRGRFTISRDNSRNTLYLQ | |
| | | MNSLRPEDTALYYCTIGGSLSRSSQGTLV | |
| | | TVSS | |
| TPP-134277 | 231 | QVQLVESGGGVVKPGGSLRLSCAASGF | HSA006A06(Q13K, K43R, K64R, |
| | | TFRSFGMSWVRQAPGRGPEWVSSISGS | K75R, K83R) |
| | | GSDTLYADSVRGRFTISRDNSRNTLYLQ | |
| | | MNSLRPEDTALYYCTIGGSLSRSSQGTLV | |
| | | TVSS | |
| TPP-134278 | 232 | QVQLVESGGGVVQPGGSLRLSCAASGF | HSA006A06(K64R, K75R, K83R) |
| | | TFRSFGMSWVRQAPGKGPEWVSSISGS | |
| | | GSDTLYADSVRGRFTISRDNSRNTLYLQ | |
| | | MNSLRPEDTALYYCTIGGSLSRSSQGTLV | |
| | | TVSS | |
| TPP-134279 | 233 | QVQLVESGGGVVQPGGSLRLSCAASGF | HSA006A06(K43R, K64R, K83R) |
| | | TFRSFGMSWVRQAPGRGPEWVSSISGS | |
| | | GSDTLYADSVRGRFTISRDNSKNTLYLQ | |
| | | MNSLRPEDTALYYCTIGGSLSRSSQGTLV | |
| | | TVSS | |
| TPP-134280 | 234 | QVQLKESGGGVVQPGGSLRLSCAASGF | HSA006A06(E1Q, V5K, K43R, |
| | | TFRSFGMSWVRQAPGRGPEWVSSISGS | K64R, A74S, K75R, T76N, |
| | | GSDTLYADSVRGRFTISRDNSRNTLYLQ | K83R,) |
| | | MNSLRPEDTALYYCTIGGSLSRSSQGTLV | |
| | | TVSS | |
| TPP-134281 | 235 | QVQLVEKGGGVVQPGGSLRLSCAASGF | HSA006A06(S7K, K43R, K64R, |
| | | TFRSFGMSWVRQAPGRGPEWVSSISGS | K75R, K83R) |
| | | GSDTLYADSVRGRFTISRDNSRNTLYLQ | |
| | | MNSLRPEDTALYYCTIGGSLSRSSQGTLV | |
| | | TVSS | |
| TPP-134282 | 236 | QVQLVESGGGVKQPGGSLRLSCAASGF | HSA006A06(V12K, K43R, K64R, |
| | | TFRSFGMSWVRQAPGRGPEWVSSISGS | K75R, K83R) |
| | | GSDTLYADSVRGRFTISRDNSRNTLYLQ | |
| | | MNSLRPEDTALYYCTIGGSLSRSSQGTLV | |
| | | TVSS | |
| TPP-134283 | 237 | QVQLVESGGGVVQPGGSLKLSCAASGF | HSA006A06(R19K, K43R, K64R, |
| | | TFRSFGMSWVRQAPGRGPEWVSSISGS | A74S, K75R, K83) |
| | | GSDTLYADSVRGRFTISRDNSRNTLYLQ | |
| | | MNSLRPEDTALYYCTIGGSLSRSSQGTLV | |
| | | TVSS | |
| TPP-134284 | 238 | QVQLVESGGGVVQPGGSLRLSCAASGF | HSA006A06(K43R, E44K, K64R, |
| | | TFRSFGMSWVRQAPGRKPEWVSSISGS | A74S, K75R, K83) |
| | | GSDTLYADSVRGRFTISRDNSRNTLYLQ | |
| | | MNSLRPEDTALYYCTIGGSLSRSSQGTLV | |
| | | TVSS | |
| TPP-134285 | 239 | QVQLVESGGGVVQPGGSLRLSCAASGF | HSA006A06(K43R, P45K, K64R, |
| | | TFRSFGMSWVRQAPGRGKEWVSSISGS | K75R, K83R) |
| | | GSDTLYADSVRGRFTISRDNSRNTLYLQ | |
| | | MNSLRPEDTALYYCTIGGSLSRSSQGTLV | |
| | | TVSS | |
| TPP-134286 | 240 | QVQLVESGGGVVQPGGSLRLSCAASGF | HSA006A06(K43R, D61K, K64R, |
| | | TFRSFGMSWVRQAPGRGPEWVSSISGS | K75R, K83R) |
| | | GSDTLYAKSVRGRFTISRDNSRNTLYLQ | |
| | | MNSLRPEDTALYYCTIGGSLSRSSQGTLV | |
| | | TVSS | |
| TPP-134287 | 241 | QVQLVESGGGVVQPGGSLRLSCAASGF | HSA006A06(K43R, K75R, K83R) |
| | | TFRSFGMSWVRQAPGRGPEWVSSISGS | |
| | | GSDTLYADSVKGRFTISRDNSRNTLYLQ | |
| | | MNSLRPEDTALYYCTIGGSLSRSSQGTLV | |
| | | TVSS | |
| TPP-134288 | 242 | QVQLVESGGGVVQPGGSLRLSCAASGF | HSA006A06(K43R, |
| | | TFRSFGMSWVRQAPGRGPEWVSSISGS | K64R, S70K, K75R, K83R) |
| | | GSDTLYADSVRGRFTIKRDNSRNTLYLQ | |
| | | MNSLRPEDTALYYCTIGGSLSRSSQGTLV | |
| | | TVSS | |
| TPP-134289 | 243 | QVQLVESGGGVVQPGGSLRLSCAASGF | HSA006A06(K43R, K64R, R71K, |
| | | TFRSFGMSWVRQAPGRGPEWVSSISGS | K75R, K83R) |
| | | GSDTLYADSVRGRFTISKDNSRNTLYLQ | |
| | | MNSLRPEDTALYYCTIGGSLSRSSQGTLV | |
| | | TVSS | |
| TPP-134290 | 244 | QVQLVESGGGVVQPGGSLRLSCAASGF | HSA006A06(K43R, K64R, N73K, |
| | | TFRSFGMSWVRQAPGRGPEWVSSISGS | K75R, K83R) |
| | | GSDTLYADSVRGRFTISRDKSRNTLYLQ | |
| | | MNSLRPEDTALYYCTIGGSLSRSSQGTLV | |
| | | TVSS | |
| TPP-134291 | 245 | QVQLVESGGGVVQPGGSLRLSCAASGF | HSA006A06(K43R, K64R, |
| | | TFRSFGMSWVRQAPGRGPEWVSSISGS | K75R) |
| | | GSDTLYADSVRGRFTISRDNSRNTLYLQ | |
| | | MNSLKPEDTALYYCTIGGSLSRSSQGTLV | |
| | | TVSS | |
| TPP-134292 | 246 | QVQLVESGGGVVQPGGSLRLSCAASGF | HSA006A06(K43R, K64R, |
| | | TFRSFGMSWVRQAPGRGPEWVSSISGS | K75R, K83R, Q105K) |
| | | GSDTLYADSVRGRFTISRDNSRNTLYLQ | |
| | | MNSLRPEDTALYYCTIGGSLSRSSKGTLV | |
| | | TVSS | |
| TPP-134293 | 247 | QVQLVESGGGVVQPGGSLRLSCAASGF | HSA006A06(K43R, K64R, |
| | | TFRSFGMSWVRQAPGRGPEWVSSISGS | K75R, K83R, Q108K) |
| | | GSDTLYADSVRGRFTISRDNSRNTLYLQ | |
| | | MNSLRPEDTALYYCTIGGSLSRSSQGTK | |
| | | VTVSS | |
| TPP-134272 | 248 | QVQLVESGGGVVQPGGSLRLSCAASGF | |
| | | TFRSFGMSWVRQAPGRGPEWVSSISGS | |
| | | GSDTLYADSVRGRFTISRDNSRNTLYLQ | |
| | | MNSLRPEDTALYYCTIGGSLSRSSQGTLV | |
| | | TVSSK | |
| TPP-134273 | 249 | GQVQLVESGGGVVQPGGSLRLSCAASG | |
| | | FTFRSFGMSWVRQAPGRGPEWVSSISG | |
| | | SGSDTLYADSVRGRFTISRDNSRNTLYLQ | |
| | | MNSLRPEDTALYYCTIGGSLSRSSQGTLV | |
| | | TVSS | |

In another embodiment, the ISVD of the present technology comprises three complementarity determining regions (CDR1 to CDR3, respectively), wherein:

CDR1 comprises the amino acid sequence of SEQ ID NO: 88 (GFTFSTADMG) (or has 3, 2 or 1 amino acid difference(s) with SEQ ID NO: 88);
CDR2 comprises the amino acid sequence of SEQ ID NO: 89 (RISGIDGTTY) (or has 3, 2 or 1 amino acid difference(s) with SEQ ID NO: 89); and
CDR3 comprises the amino acid sequence of SEQ ID NO: 90 (PRYADQWSAYDY) (or has 3, 2 or 1 amino acid difference(s) with SEQ ID NO: 90);

wherein the CDR sequences are determined according to AbM definition.

In another embodiment, the ISVD of the present technology comprises three complementarity determining regions (CDR1 to CDR3, respectively), wherein:
CDR1 comprises the amino acid sequence of SEQ ID NO: 270 (TADMG) (or has 3, 2 or 1 amino acid difference(s) with SEQ ID NO: 270);
CDR2 comprises the amino acid sequence of SEQ ID NO: 271 (RISGIDGTTYYDEPVKG) (or has 3, 2 or 1 amino acid difference(s) with SEQ ID NO: 271); and
CDR3 comprises the amino acid sequence of SEQ ID NO: 90 (PRYADQWSAYDY) (or has 3, 2 or 1 amino acid difference(s) with SEQ ID NO: 90);
wherein the CDR sequences are determined according to Kabat numbering.

Preferably, the at least one ISVD may be selected from an ISVD comprising or consisting of SEQ ID NO.: 69-78, see Table 7, or a sequence which has at least 80%, such as at least 85%, or at least 90% or at least 95%, or at least 99% identity with a sequence as defined in SEQ IS NO.: 69-78.

TABLE 7
Sequence of TNF alpha-binding ISVDs derived from
TNF006C11 (positions in the description are
numbered according to Kabat) - Precursor is
A016600058

| | | | Description |
|---|---|---|---|
| | SEQ | | (Mutations |
| | ID | | related to |
| ID | NO | Amino acid sequence | invention) |
| T031200256 | 69 | DVQLVESGGGVVQPGGSLRL | TNF006C11 |
| | | SCTASGFTFSTADMGWFRQA | (Q75K) |
| | | PGKGREFVARISGIDGTTYY | |
| | | DEPVKGRFTISRDKSKNTVY | |
| | | LQMNSLKPEDTALYYCRSPR | |
| | | YADQWSAYDYWGQGTLVTVS | |
| | | S | |
| T031200257 | 70 | DVQLVESGGGVVQPGGSLKL | TNF006C11 |
| | | SCTASGFTFSTADMGWFRQA | (R19K, |
| | | PGKGREFVARISGIDGTTYY | Q75K) |
| | | DEPVKGRFTISRDKSKNTVY | |
| | | LQMNSLKPEDTALYYCRSPR | |
| | | YADQWSAYDYWGQGTLVTVS | |
| | | S | |
| T031200258 | 71 | DVQLVESGGGVVQPGGSLKL | TNF006C11 |
| | | SCTASGFTFSTADMGWFRQA | (R19K, |
| | | PGKGREFVARISGIDGTTYY | K73N, |
| | | DEPVKGRFTISRDNSKNTVY | Q75K, |
| | | LQMNSLKPEDTALYYCRSPR | Q105K) |
| | | YADQWSAYDYWGKGTLVTVS | |
| | | S | |
| T031200259 | 72 | DVQLVESGGGVVQPGGSLKL | TNF006C11 |
| | | SCTASGFTFSTADMGWFRQA | (R19K, |
| | | PGKGREFVARISGIDGTTYY | R71K, |
| | | DEPVKGRFTISKDNSKNTVY | K73N, |
| | | LQMNSLKPEDTALYYCRSPR | Q75K, |
| | | YADQWSAYDYWGKGTLVTVS | Q105K) |
| | | S | |
| T031200260 | 73 | DVQLVESGGGVVQPGGSLKL | TNF006C11 |
| | | SCTASGFTFSTADMGWFRQA | (R19K, |
| | | PGKGREFVARISGIDGTTYY | Q75K, |
| | | DEPVKGRFTISRDKSKNTVY | Q105K) |
| | | LQMNSLKPEDTALYYCRSPR | |
| | | YADQWSAYDYWGKGTLVTVS | |
| | | S | |
| T031200261 | 74 | DVQLVESGGGVVQPGGSLKL | TNF006C11 |
| | | SCTASGFTFSTADMGWFRQA | (R19K, |
| | | PGKGREFVARISGIDGTTYY | R71K, |
| | | DEPVKGRFTISKDKSKNTVY | Q75K, |
| | | LQMNSLKPEDTALYYCRSPR | Q105K) |
| | | YADQWSAYDYWGKGTLVTVS | |
| | | S | |
| T031200262 | 75 | DVQLVESGGGVVQPGGSLKL | TNF006C11 |
| | | SCTASGFTFSTADMGWFRQA | (R19K, |
| | | PGKGKEFVARISGIDGTTYY | R45K, |
| | | DEPVKGRFTISRDKSKNTVY | Q75K, |
| | | LQMNSLKPEDTALYYCRSPR | Q105K) |
| | | YADQWSAYDYWGKGTLVTVS | |
| | | S | |
| T031200263 | 78 | DVQLVESGGGVVQPGGSLKL | TNF006C11 |
| | | SCTASGFTFSTADMGWFRQA | (R19K, |
| | | PGKGKEFVARISGIDGTTYY | R45K, |
| | | DEPVKGRFTISKDKSKNTVY | R71K, |
| | | LQMNSLKPEDTALYYCRSPR | Q75K, |
| | | YADQWSAYDYWGKGTLVTVS | Q105K) |
| | | S | |

In another embodiment, the ISVD of the present technology comprises three complementarity determining regions (CDR1 to CDR3, respectively), wherein:
CDR1 comprises the amino acid sequence of SEQ ID NO: 251 (GRTSRSYGMG) (or has 3, 2 or 1 amino acid difference(s) with SEQ ID NO: 251);
CDR2 comprises the amino acid sequence of SEQ ID NO: 252 (GISWRGDSTG) (or has 3, 2 or 1 amino acid difference(s) with SEQ ID NO: 252); and
CDR3 comprises the amino acid sequence of SEQ ID NO: 253 (AAGSAWYGTLYEYDY) (or has 3, 2 or 1 amino acid difference(s) with SEQ ID NO: 253);

wherein the CDR sequences are determined according to AbM definition.

In another embodiment, the ISVD of the present technology comprises three complementarity determining regions (CDR1 to CDR3, respectively), wherein:
CDR1 comprises the amino acid sequence of SEQ ID NO: 272 (SYGMG) (or has 3, 2 or 1 amino acid difference(s) with SEQ ID NO: 272);
CDR2 comprises the amino acid sequence of SEQ ID NO: 273 (GISWRGDSTGYADSVRG) (or has 3, 2 or 1 amino acid difference(s) with SEQ ID NO: 273); and
CDR3 comprises the amino acid sequence of SEQ ID NO: 253 (AAGSAWYGTLYEYDY) (or has 3, 2 or 1 amino acid difference(s) with SEQ ID NO: 253);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 272 (SYGMG) (or has 3, 2 or 1 amino acid difference(s) with SEQ ID NO: 272);
CDR2 comprises the amino acid sequence of SEQ ID NO: 305 (GISWRGDSTGYADSVKG) (or has 3, 2 or 1 amino acid difference(s) with SEQ ID NO: 305); and
CDR3 comprises the amino acid sequence of SEQ ID NO: 253 (AAGSAWYGTLYEYDY) (or has 3, 2 or 1 amino acid difference(s) with SEQ ID NO: 253);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 272 (SYGMG) (or has 3, 2 or 1 amino acid difference(s) with SEQ ID NO: 272);
CDR2 comprises the amino acid sequence of SEQ ID NO: 306 (GISWRGDSTGYAKSVRG) (or has 3, 2 or 1 amino acid difference(s) with SEQ ID NO: 306); and
CDR3 comprises the amino acid sequence of SEQ ID NO: 253 (AAGSAWYGTLYEYDY) (or has 3, 2 or 1 amino acid difference(s) with SEQ ID NO: 253);
wherein the CDR sequences are determined according to Kabat numbering.

Preferably, the at least one ISVD may be selected from an ISVD comprising or consisting of SEQ ID NO.: 173-194, see Table 8, or a sequence which has at least 80%, such as at least 85%, or at least 90% or at least 95%, or at least 99% identity with a sequence as defined in SEQ IS NO.: 173-194.

TABLE 8
Sequence of EGFR-binding ISVDs derived from EGFR007D12 (positions
in the description are numbered according to Kabat) - Precursor
is TPP-134228

| | SEQ | | |
|---|---|---|---|
| | ID | | Description (Mutations |
| ID | NO | Amino acid sequence | related to invention) |
| TPP-134228 | 173 | QVQLEESGGGSVQTGGSLRLTCAASGRTS | EGFR007D12(K3Q) |
| | | RSYGMGWFRQAPGKEREFVSGISWRGD | |
| | | STGYADSVKGRFTISRDNAKNTVDLQMN | |
| | | SLKPEDTAIYYCAAAAGSAWYGTLYEYDY | |
| | | WGQGTQVTVSS | |
| TPP-134229 | 174 | GQVQLEESGGGSVQTGGSLRLTCAASGR | G- |
| | | TSRSYGMGWFRQAPGREREFVSGISWR | EGFR007D12(K3Q, K43R, |
| | | GDSTGYADSVRGRFTISRDNARNTVDLQ | K64R, K75R, K83R) |
| | | MNSLRPEDTAIYYCAAAAGSAWYGTLYEY | |
| | | DYWGQGTQVTVSS | |
| TPP-134230 | 175 | QVQLEESGGGSVQTGGSLRLTCAASGRTS | EGFR007D12(K3Q, K43R, |
| | | RSYGMGWFRQAPGREREFVSGISWRGD | K64R, K75R, K83R) |
| | | STGYADSVRGRFTISRDNARNTVDLQMN | |
| | | SLRPEDTAIYYCAAAAGSAWYGTLYEYDY | |
| | | WGQGTQVTVSS | |
| TPP-134231 | 176 | QVQLEESGGGSVQTGGSLRLTCAASGRTS | EGFR007D12(K3Q, K43R, |
| | | RSYGMGWFRQAPGREREFVSGISWRGD | K64R, K75R, K83R)-K |
| | | STGYADSVRGRFTISRDNARNTVDLQMN | |
| | | SLRPEDTAIYYCAAAAGSAWYGTLYEYDY | |
| | | WGQGTQVTVSSK | |
| TPP-134232 | 177 | QVQLEESGGGSVQTGGSLKLTCAASGRTS | EGFR007D12(K3Q, R19K, |
| | | RSYGMGWFRQAPGREREFVSGISWRGD | K43R, K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134233 | 178 | QVQLEESGGKSVQTGGSLRLTCAASGRTS | EGFR007D12(K3Q, G10K, |
| | | RSYGMGWFRQAPGREREFVSGISWRGD | K43R, K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134234 | 179 | QVQLEESGGGSVQTGGSLRLTCAASGRTS | EGFR007D12(K3Q, K43R, |
| | | RSYGMGWFRQAPGREREFVSGISWRGD | K64R,K75R, K83R, |
| | | STGYADSVRGRFTISRDNARNTVDLQMN | Q105K) |
| | | SLRPEDTAIYYCAAAAGSAWYGTLYEYDY | |
| | | WGKGTQVTVSS | |
| TPP-134235 | 180 | QVQLEESGGGSVQTGGSLRLTCAASGRTS | EGFR007D12(K3Q, K43R, |
| | | RSYGMGWFRQAPGREREFVSGISWRGD | K64R, K75R, K83R, |
| | | STGYADSVRGRFTISRDNARNTVDLQMN | Q108K) |
| | | SLRPEDTAIYYCAAAAGSAWYGTLYEYDY | |
| | | WGQGTKVTVSS | |
| TPP-134236 | 181 | QVQLEESGGGSKQTGGSLRLTCAASGRTS | EGFR007D12(K3Q, V12K, |
| | | RSYGMGWFRQAPGREREFVSGISWRGD | K43R, K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134237 | 182 | QVQLEESGGGSVKTGGSLRLTCAASGRTS | EGFR007D12(K3Q, Q13K, |
| | | RSYGMGWFRQAPGREREFVSGISWRGD | K43R,K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134238 | 183 | QVQLEESGGGSVQTGGSLRLTCAASGRTS | EGFR007D12(K3Q, K64R, |
| | | RSYGMGWFRQAPGKEREFVSGISWRGD | K75R, K83R) |
| | | STGYADSVRGRFTISRDNARNTVDLQMN | |
| | | SLRPEDTAIYYCAAAAGSAWYGTLYEYDY | |
| | | WGQGTQVTVSS | |
| TPP-134239 | 184 | QVQLEESGGGSVQTGGSLRLTCAASGRTS | EGFR007D12(K3Q, K43R, |
| | | RSYGMGWFRQAPGRKREFVSGISWRGD | E44K, K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134240 | 185 | QVQLEESGGGSVQTGGSLRLTCAASGRTS | EGFR007D12(K3Q, K43R, |
| | | RSYGMGWFRQAPGREKEFVSGISWRGD | R45K,K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134241 | 186 | QVQLKESGGGSVQTGGSLRLTCAASGRTS | EGFR007D12(K3Q, E5K, |
| | | RSYGMGWFRQAPGREREFVSGISWRGD | K43R,K64R, K75R, |
| | | STGYADSVRGRFTISRDNARNTVDLQMN | K83R) |
| | | SLRPEDTAIYYCAAAAGSAWYGTLYEYDY | |
| | | WGQGTQVTVSS | |
| TPP-134242 | 187 | QVQLEESGGGSVQTGGSLRLTCAASGRTS | EGFR007D12(K3Q, K43R, |
| | | RSYGMGWFRQAPGREREFVSGISWRGD | D61K, K64R, K75R, |
| | | STGYAKSVRGRFTISRDNARNTVDLQMN | K83R) |
| | | SLRPEDTAIYYCAAAAGSAWYGTLYEYDY | |
| | | WGQGTQVTVSS | |
| TPP-134243 | 188 | QVQLEESGGGSVQTGGSLRLTCAASGRTS | EGFR007D12(K3Q, K43R, |
| | | RSYGMGWFRQAPGREREFVSGISWRGD | K75R, K83R) |
| | | STGYADSVKGRFTISRDNARNTVDLQMN | |
| | | SLRPEDTAIYYCAAAAGSAWYGTLYEYDY | |
| | | WGQGTQVTVSS | |
| TPP-134244 | 189 | QVQLEEKGGGSVQTGGSLRLTCAASGRT | EGFR007D12(K3Q, S7K, |
| | | SRSYGMGWFRQAPGREREFVSGISWRG | K43R, K64R, K75R, |
| | | DSTGYADSVRGRFTISRDNARNTVDLQM | K83R) |
| | | NSLRPEDTAIYYCAAAAGSAWYGTLYEYD | |
| | | YWGQGTQVTVSS | |
| TPP-134245 | 190 | QVQLEESGGGSVQTGGSLRLTCAASGRTS | EGFR007D12(K3Q, K43R, |
| | | RSYGMGWFRQAPGREREFVSGISWRGD | K64R,S70K, K75R, |
| | | STGYADSVRGRFTIKRDNARNTVDLQMN | K83R) |
| | | SLRPEDTAIYYCAAAAGSAWYGTLYEYDY | |
| | | WGQGTQVTVSS | |
| TPP-134246 | 191 | QVQLEESGGGSVQTGGSLRLTCAASGRTS | EGFR007D12(K3Q, K43R, |
| | | RSYGMGWFRQAPGREREFVSGISWRGD | K64R,R71K, K75R, |
| | | STGYADSVRGRFTISKDNARNTVDLQMN | K83R) |
| | | SLRPEDTAIYYCAAAAGSAWYGTLYEYDY | |
| | | WGQGTQVTVSS | |
| TPP-134247 | 192 | QVQLEESGGGSVQTGGSLRLTCAASGRTS | EGFR007D12(K3Q, K43R, |
| | | RSYGMGWFRQAPGREREFVSGISWRGD | K64R,N73K, K75R, |
| | | STGYADSVRGRFTISRDKARNTVDLQMN | K83R) |
| | | SLRPEDTAIYYCAAAAGSAWYGTLYEYDY | |
| | | WGQGTQVTVSS | |
| TPP-134248 | 193 | QVQLEESGGGSVQTGGSLRLTCAASGRTS | EGFR007D12(K3Q, K43R, |
| | | RSYGMGWFRQAPGREREFVSGISWRGD | K64R, K83R) |
| | | STGYADSVRGRFTISRDNAKNTVDLQMN | |
| | | SLRPEDTAIYYCAAAAGSAWYGTLYEYDY | |
| | | WGQGTQVTVSS | |
| TPP-134249 | 194 | QVQLEESGGGSVQTGGSLRLTCAASGRTS | EGFR007D12(K3Q, K43R, |
| | | RSYGMGWFRQAPGREREFVSGISWRGD | K64R, K75R) |
| | | STGYADSVRGRFTISRDNARNTVDLQMN | |
| | | SLKPEDTAIYYCAAAAGSAWYGTLYEYDY | |
| | | WGQGTQVTVSS | |

In another embodiment, the ISVD of the present technology comprises three complementarity determining regions (CDR1 to CDR3, respectively), wherein:
CDR1 comprises the amino acid sequence of SEQ ID NO: 254 (GLTFSTNPMY) (or has 3, 2 or 1 amino acid difference(s) with SEQ ID NO: 254);
CDR2 comprises the amino acid sequence of SEQ ID NO: 255 (SISSRGITN) (or has 3, 2 or 1 amino acid difference(s) with SEQ ID NO: 255); and
CDR3 comprises the amino acid sequence of SEQ ID NO: 256 (ASLSSGTVY) (or has 3, 2 or 1 amino acid difference(s) with SEQ ID NO: 256);
wherein the CDR sequences are determined according to AbM numbering.

In another embodiment, the ISVD of the present technology comprises three complementarity determining regions (CDR1 to CDR3, respectively), wherein:
CDR1 comprises the amino acid sequence of SEQ ID NO: 274 (TNPMY) (or has 3, 2 or 1 amino acid difference(s) with SEQ ID NO: 274);
CDR2 comprises the amino acid sequence of SEQ ID NO: 275 (SISSRGITNYADSVRG) (or has 3, 2 or 1 amino acid difference(s) with SEQ ID NO: 275); and
CDR3 comprises the amino acid sequence of SEQ ID NO: 256 (ASLSSGTVY) (or has 3, 2 or 1 amino acid difference(s) with SEQ ID NO: 256);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 274 (TNPMY) (or has 3, 2 or 1 amino acid difference(s) with SEQ ID NO: 274);
CDR2 comprises the amino acid sequence of SEQ ID NO: 296 (SISSRGITNYADSVKG) (or has 3, 2 or 1 amino acid difference(s) with SEQ ID NO: 296); and
CDR3 comprises the amino acid sequence of SEQ ID NO: 256 (ASLSSGTVY) (or has 3, 2 or 1 amino acid difference(s) with SEQ ID NO: 256);

wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 274 (TNPMY) (or has 3, 2 or 1 amino acid difference(s) with SEQ ID NO: 274);
CDR2 comprises the amino acid sequence of SEQ ID NO: 297 (SISSRGITNYAKSVRG) (or has 3, 2 or 1 amino acid difference(s) with SEQ ID NO: 297); and
CDR3 comprises the amino acid sequence of SEQ ID NO: 256 (ASLSSGTVY) (or has 3, 2 or 1 amino acid difference(s) with SEQ ID NO: 256);
5 wherein the CDR sequences are determined according to Kabat numbering.

Preferably, the at least one ISVD may be selected from an ISVD comprising or consisting of SEQ ID NO.: 195-218, see Table 9, or a sequence which has at least 80%, such as at least 85%, or at least 90% or at least 95%, or at least 99% identity with a sequence as defined in SEQ IS NO.: 195-218.

TABLE 9
Sequence of pcrV-binding ISVDs derived from A0095013F07 (positions
in the description are numbered according to Kabat) - Precursor
is T042500044

| | SEQ | | |
|---|---|---|---|
| | ID | | Description (Mutations |
| ID | NO | Amino acid sequence | related to invention) |
| T042500044 | 195 | EVQLVESGGGLVQPGGSLRLSCAASGLT | A0095013F07(R64K, K83R, |
| | | FSTNPMYWYRQAPGKQRELVASISSRGI | K105Q) |
| | | TNYADSVKGRFTISRDNSKNTVYLQMNS | |
| | | LRPEDTAVYYCRLASLSSGTVYWGQGTL | |
| | | VTVSS | |
| TPP-134250 | 196 | QVQLVESGGGLVQPGGSLRLSCAASGLT | A0095013F07(E1Q, K43R, |
| | | FSTNPMYWYRQAPGRQRELVASISSRGI | K75R, K83R, K105Q)-K |
| | | TNYADSVRGRFTISRDNSRNTVYLQMN | |
| | | SLRPEDTAVYYCRLASLSSGTVYWGQGT | |
| | | LVTVSSK | |
| TPP-134251 | 197 | QVQLVESGGGLVQPGGSLRLSCAASGLT | A0095013F07(E1Q, R64K, |
| | | FSTNPMYWYRQAPGKQRELVASISSRGI | K83R, K105Q) |
| | | TNYADSVKGRFTISRDNSKNTVYLQMNS | |
| | | LRPEDTAVYYCRLASLSSGTVYWGQGTL | |
| | | VTVSS | |
| TPP-134252 | 198 | GQVQLVESGGGLVQPGGSLRLSCAASG | G-A0095013F07(E1Q, K43R, |
| | | LTFSTNPMYWYRQAPGRQRELVASISSR | K75R, K83R, K105Q) |
| | | GITNYADSVRGRFTISRDNSRNTVYLQM | |
| | | NSLRPEDTAVYYCRLASLSSGTVYWGQG | |
| | | TLVTVSS | |
| TPP-134253 | 199 | QVQLVESGGGLVQPGGSLRLSCAASGLT | A0095013F07(E1Q, K43R, |
| | | FSTNPMYWYRQAPGRQRELVASISSRGI | K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134254 | 200 | QVQLKESGGGLVQPGGSLRLSCAASGLT | A0095013F07(E1Q, V5K, |
| | | FSTNPMYWYRQAPGRQRELVASISSRGI | K43R, K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134255 | 201 | QVQLVEKGGGLVQPGGSLRLSCAASGLT | A0095013F07(E1Q, S7K, |
| | | FSTNPMYWYRQAPGRQRELVASISSRGI | K43R, K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134256 | 202 | QVQLVESGGKLVQPGGSLRLSCAASGLT | A0095013F07(E1Q, G10K, |
| | | FSTNPMYWYRQAPGRQRELVASISSRGI | K43R, K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134257 | 203 | QVQLVESGGGLKQPGGSLRLSCAASGLT | A0095013F07(E1Q, V12K, |
| | | FSTNPMYWYRQAPGRQRELVASISSRGI | K43R, K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134258 | 204 | QVQLVESGGGLVKPGGSLRLSCAASGLT | A0095013F07(E1Q, Q13K, |
| | | FSTNPMYWYRQAPGRQRELVASISSRGI | K43R, K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134259 | 205 | QVQLVESGGGLVQPGGSLKLSCAASGLT | A0095013F07(E1Q,Q13K, |
| | | FSTNPMYWYRQAPGRQRELVASISSRGI | K43R,K75R, K83R,K105Q) |
| | | TNYADSVRGRFTISRDNSRNTVYLQMN | |
| | | SLRPEDTAVYYCRLASLSSGTVYWGQGT | |
| | | LVTVSS | |
| TPP-134260 | 206 | QVQLVESGGGLVQPGGSLRLSCAASGLT | A0095013F07(E1Q, K75R, |
| | | FSTNPMYWYRQAPGKQRELVASISSRGI | K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134261 | 207 | QVQLVESGGGLVQPGGSLRLSCAASGLT | A0095013F07(E1Q, K43R, |
| | | FSTNPMYWYRQAPGRKRELVASISSRGI | Q44K, K75R, K83R, K105Q) |
| | | TNYADSVRGRFTISRDNSRNTVYLQMN | |
| | | SLRPEDTAVYYCRLASLSSGTVYWGQGT | |
| | | LVTVSS | |
| TPP-134262/ | 208 | QVQLVESGGGLVQPGGSLRLSCAASGLT | A0095013F07(E1Q, K43R, |
| T032301000 | | FSTNPMYWYRQAPGRQKELVASISSRGI | R45K, K75R, K83R, K105Q) |
| 34 | | TNYADSVRGRFTISRDNSRNTVYLQMN | |
| | | SLRPEDTAVYYCRLASLSSGTVYWGQGT | |
| | | LVTVSS | |
| TPP-134263 | 209 | QVQLVESGGGLVQPGGSLRLSCAASGLT | A0095013F07(E1Q, K43R, |
| | | FSTNPMYWYRQAPGRQRELVASISSRGI | D61K, K75R, K83R, K105Q) |
| | | TNYAKSVRGRFTISRDNSRNTVYLQMNS | |
| | | LRPEDTAVYYCRLASLSSGTVYWGQGTL | |
| | | VTVSS | |
| TPP-134264 | 210 | QVQLVESGGGLVQPGGSLRLSCAASGLT | A0095013F07(E1Q, K43R, |
| | | FSTNPMYWYRQAPGRQRELVASISSRGI | R64K, K75R, K83R, K105Q) |
| | | TNYADSVKGRFTISRDNSRNTVYLQMNS | |
| | | LRPEDTAVYYCRLASLSSGTVYWGQGTL | |
| | | VTVSS | |
| TPP-134265 | 211 | QVQLVESGGGLVQPGGSLRLSCAASGLT | A0095013F07(E1Q, K43R, |
| | | FSTNPMYWYRQAPGRQRELVASISSRGI | S70K, T74S, K75R, K83R, |
| | | TNYADSVRGRFTIKRDNSRNTVYLQMN | K105Q) |
| | | SLRPEDTAVYYCRLASLSSGTVYWGQGT | |
| | | LVTVSS | |
| TPP-134266 | 212 | QVQLVESGGGLVQPGGSLRLSCAASGLT | A0095013F07(E1Q, K43R, |
| | | FSTNPMYWYRQAPGRQRELVASISSRGI | R71K, K75R, K83R, K105Q) |
| | | TNYADSVRGRFTISKDNSRNTVYLQMNS | |
| | | LRPEDTAVYYCRLASLSSGTVYWGQGTL | |
| | | VTVSS | |
| TPP-134267 | 213 | QVQLVESGGGLVQPGGSLRLSCAASGLT | A0095013F07(E1Q, K43R, |
| | | FSTNPMYWYRQAPGRQRELVASISSRGI | D71K, K75R, K83R, K105Q) |
| | | TNYADSVRGRFTISRDKSRNTVYLQMNS | |
| | | LRPEDTAVYYCRLASLSSGTVYWGQGTL | |
| | | VTVSS | |
| TPP-134268 | 214 | QVQLVESGGGLVQPGGSLRLSCAASGLT | A0095013F07(E1Q, K43R, |
| | | FSTNPMYWYRQAPGRQRELVASISSRGI | K83R, K105Q) |
| | | TNYADSVRGRFTISRDNSKNTVYLQMNS | |
| | | LRPEDTAVYYCRLASLSSGTVYWGQGTL | |
| | | VTVSS | |
| TPP-134269 | 215 | QVQLVESGGGLVQPGGSLRLSCAASGLT | A0095013F07(E1Q, K43R, |
| | | FSTNPMYWYRQAPGRQRELVASISSRGI | K75R, D76N, K105Q) |
| | | TNYADSVRGRFTISRDNSRNTVYLQMN | |
| | | SLKPEDTAVYYCRLASLSSGTVYWGQGT | |
| | | LVTVSS | |
| TPP-134270 | 216 | QVQLVESGGGLVQPGGSLRLSCAASGLT | A0095013F07(E1Q, K43R, |
| | | FSTNPMYWYRQAPGRQRELVASISSRGI | K75R, K83R) |
| | | TNYADSVRGRFTISRDNSRNTVYLQMN | |
| | | SLRPEDTAVYYCRLASLSSGTVYWGKGT | |
| | | LVTVSS | |
| TPP-134271 | 217 | QVQLVESGGGLVQPGGSLRLSCAASGLT | A0095013F07(E1Q, K43R, |
| | | FSTNPMYWYRQAPGRQRELVASISSRGI | K75R, K83R, K105Q, L108K) |
| | | TNYADSVRGRFTISRDNSRNTVYLQMN | |
| | | SLRPEDTAVYYCRLASLSSGTVYWGQGT | |
| | | KVTVSS | |
| T032301000 | 218 | QVQLVESGGGLVQPGGSLRLSCAASGLT | A0095013F07(E1Q, K43R, |
| 35 | | FSTNPMYWYRQAPGRQKELVASISSRGI | R45K, N73K, K75R, K83R, |
| | | TNYADSVRGRFTISRDKSRNTVYLQMNS | K105Q) |
| | | LRPEDTAVYYCRLASLSSGTVYWGQGTL | |
| | | VTVSS | |

In another embodiment, the ISVD of the present technology comprises three complementarity determining regions (CDR1 to CDR3, respectively), wherein:
CDR1 comprises the amino acid sequence of SEQ ID NO: 257 (GRTFSSYAMG) (or has 3, 2 or 1 amino acid difference(s) with SEQ ID NO: 257);
CDR2 comprises the amino acid sequence of SEQ ID NO: 258 (AISWSDGSTY) (or has 3, 2 or 1 amino acid difference(s) with SEQ ID NO: 258); and
CDR3 comprises the amino acid sequence of SEQ ID NO: 259 (DLTSTNPGSYIYIWAYDY) (or has 3, 2 or 1 amino acid difference(s) with SEQ ID NO: 259);

wherein the CDR sequences are determined according to AbM numbering.

In another embodiment, the ISVD of the present technology comprises three complementarity determining regions (CDR1 to CDR3, respectively), wherein:
CDR1 comprises the amino acid sequence of SEQ ID NO: 298 (SYAMG) (or has 3, 2 or 1 amino acid difference(s) with SEQ ID NO: 298);
CDR2 comprises the amino acid sequence of SEQ ID NO: 299 (AISWSDGSTYYADSVRG) (or has 3, 2 or 1 amino acid difference(s) with SEQ ID NO: 299); and
CDR3 comprises the amino acid sequence of SEQ ID NO: 259 (DLTSTNPGSYIYIWAYDY) (or has 3, 2 or 1 amino acid difference(s) with SEQ ID NO: 259);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 298 (SYAMG) (or has 3, 2 or 1 amino acid difference(s) with SEQ ID NO: 298);
CDR2 comprises the amino acid sequence of SEQ ID NO: 230 (AISWSDGSTYYADSVKG) (or has 3, 2 or 1 amino acid difference(s) with SEQ ID NO: 230); and
CDR3 comprises the amino acid sequence of SEQ ID NO: 259 (DLTSTNPGSYIYIWAYDY) (or has 3, 2 or 1 amino acid difference(s) with SEQ ID NO: 259);
wherein the CDR sequences are determined according to Kabat numbering.

Preferably, the at least one ISVD may be selected from an ISVD comprising or consisting of SEQ ID NO.: 219-223, see Table 10, or a sequence which has at least 80%, such as at least 85%, or at least 90% or at least 95%, or at least 99% identity with a sequence as defined in SEQ IS NO.: 219-223.

TABLE 10
Sequence of RSV protein F-binding ISVDs
derived from RSV007B02 (positions in the
description are numbered according to
Kabat) - Precursor is T042500096

| | | | Description |
|---|---|---|---|
| | SEQ | | (Mutations |
| | ID | | related to |
| ID | NO | Amino acid sequence | invention) |
| T042500096 | 219 | EVQLVESGGGVVQPGGSLRL | RSV007B02 |
| | | SCAASGRTFSSYAMGWFRQA | (K83R) |
| | | PGKEREFVAAISWSDGSTYY | |
| | | ADSVKGRFTISRDNAKNTVY | |
| | | LQMNSLRPEDTALYYCAADL | |
| | | TSTNPGSYIYIWAYDYWGQG | |
| | | TLVTVSS | |
| T03230100013 | 220 | QVQLVESGGGVVQPGGSLRL | RSV007B02 |
| | | SCAASGRTFSSYAMGWFRQA | (E1Q, |
| | | PGREKEFVAAISWSDGSTYY | K43R, |
| | | ADSVRGRFTISRDNARNTVY | R45K, |
| | | LQMNSLRPEDTALYYCAADL | K64R, |
| | | TSTNPGSYIYIWAYDYWGQG | K75R, |
| | | TKVTVSS | K83R, |
| | | | Q108K) |
| T03230100014 | 221 | QVQLVESGGGVVQPGGSLRL | RSV007B02 |
| | | SCAASGRTFSSYAMGWFRQA | (E1Q, |
| | | PGREKEFVAAISWSDGSTYY | K43R, |
| | | ADSVRGRFTISRDKARNTVY | R45K, |
| | | LQMNSLRPEDTALYYCAADL | K64R, |
| | | TSTNPGSYIYIWAYDYWGQG | N73K, |
| | | TLVTVSS | K75R, |
| | | | K83R) |
| T03230100033 | 222 | QVQLVESGGGVVKPGGSLRL | RSV007B02 |
| | | SCAASGRTFSSYAMGWFRQA | (E1Q, |
| | | PGREKEFVAAISWSDGSTYY | Q13K, |
| | | ADSVRGRFTISRDNARNTVY | K43R, |
| | | LQMNSLRPEDTALYYCAADL | R45K, |
| | | TSTNPGSYIYIWAYDYWGQG | K64R, |
| | | TLVTVSS | K75R, |
| | | | K83R) |
| T03230100032 | 223 | QVQLVESGGGVVQPGGSLRL | RSV007B02 |
| | | SCAASGRTFSSYAMGWFRQA | (K43R, |
| | | PGREKEFVAAISWSDGSTYY | R45K, |
| | | ADSVRGRFTISRDNARNTVY | K64R, |
| | | LQMNSLRPEDTALYYCAADL | K75R, |
| | | TSTNPGSYIYIWAYDYWGQG | K83R) |
| | | TLVTVSS | |

In another embodiment, the ISVD of the present technology comprises three complementarity determining regions (CDR1 to CDR3, respectively), wherein:

CDR1 comprises the amino acid sequence of SEQ ID NO: 261 (GFTFEDYAIG) (or has 3, 2 or 1 amino acid difference(s) with SEQ ID NO: 261);
CDR2 comprises the amino acid sequence of SEQ ID NO: 262 (CIRTYDEQTY) (or has 3, 2 or 1 amino acid difference(s) with SEQ ID NO: 262); and
CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to AbM numbering.

In another embodiment, the ISVD of the present technology comprises three complementarity determining regions (CDR1 to CDR3, respectively), wherein:
CDR1 comprises the amino acid sequence of SEQ ID NO: 261 (GFTFEDYAIG) (or has 3, 2 or 1 amino acid difference(s) with SEQ ID NO: 261);
CDR2 comprises the amino acid sequence of SEQ ID NO: 262 (CIRTYDEQTY) (or has 3, 2 or 1 amino acid difference(s) with SEQ ID NO: 262); and
CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to AbM numbering.

In another embodiment, the ISVD of the present technology comprises three complementarity determining regions (CDR1 to CDR3, respectively), wherein:
CDR1 comprises the amino acid sequence of SEQ ID NO: 301 (DYAIG) (or has 3, 2 or 1 amino acid difference(s) with SEQ ID NO: 301);
CDR2 comprises the amino acid sequence of SEQ ID NO: 302 (CIRTYDEQTYYADSVKG) (or has 3, 2 or 1 amino acid difference(s) with SEQ ID NO: 302); and
CDR3 comprises amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to Kabat numbering.

In another embodiment, the ISVD of the present technology comprises three complementarity determining regions (CDR1 to CDR3, respectively), wherein:
CDR1 comprises the amino acid sequence of SEQ ID NO: 301 (DYAIG) (or has 3, 2 or 1 amino acid difference(s) with SEQ ID NO: 301);
CDR2 comprises the amino acid sequence of SEQ ID NO: 303 (CIRTYDEQTYYADSVRG) (or has 3, 2 or 1 amino acid difference(s) with SEQ ID NO: 303); and
CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to Kabat numbering.

Preferably, the at least one ISVD may be selected from an ISVD comprising or consisting of SEQ ID NO.: 224, 250 see Table 11, or a sequence which has at least 80%, such as at least 85%, or at least 90% or at least 95%, or at least 99% identity with a sequence as defined in SEQ IS NO.: 224,250.

TABLE 11
Sequence of CD8-binding ISVD derived
from EGFR007D12 (positions in the
description are numbered according
to Kabat) - Precursor is A044301152

| | | | Description |
|---|---|---|---|
| | SEQ | | (Mutations |
| | ID | | related to |
| ID | NO | Amino acid sequence | invention) |
| A044301152 | 250 | EVQLVESGGGVVQPGGSLRL | T0347015C01 |
| | | SCAASGFTFEDYAIGWFRQA | (K83R) |
| | | PGKEREEVSCIRTYDEQTYY | |
| | | ADSVKGRFTISRDNAKNTVS | |
| | | LQMNSLRPEDTALYYCAAGS | |
| | | YYACAYYSRPDPSEGHVDLD | |
| | | YWGQGTLVTVSS | |
| A044301298 | 224 | EVQLVESGGGVVQPGGSLRL | T0347015C01 |
| | | SCAASGFTFEDYAIGWFRQA | (K43R, |
| | | PGREREEVSCIRTYDEQTYY | K64R, |
| | | ADSVRGRFTISRDNARNTVS | K75R, |
| | | LQMNSLRPEDTALYYCAAGS | K83R) |
| | | YYACAYYSRPDPSEGHVDLD | |
| | | YWGQGTLVTVSS | |

The ISVD precursor may be any ISVD. For instance, the ISVD precursor may be selected from the ISVDs with SEQ ID NOs.: 1, 26, 43,68, 225-227 and 250, see Table 12.

TABLE 12
Sequence of ISVD precursors (positions in the
description are numbered according to Kabat)

| | SEQ | | |
|---|---|---|---|
| | ID | | |
| ID | NO | Amino acid sequence | Description |
| A031500384/ | 1 | EVQLVESGGGVVQPGGSLRL | A0315007E07 |
| T028501789 | | SCAASGLTFSTYTMGWFRQA | (L11V, |
| | | PGKEREFVAAIIWSGSNTYY | S76N, |
| | | ADSVKGRFTISRDNAKNTVY | N82bS, |
| | | LQMNSLRPEDTALYYCAAQH | E83R, |
| | | FGPIGLTTRGYHYWGQGTLV | V89L, |
| | | TVSS | H91Y, |
| | | | N101H) |
| A031500099/ | 26 | EVQLVESGGGVVQPGGSLRL | A0315024B02 |
| T028501817 | | SCAASGHTFSEYALGWFRQA | (L11V, |
| | | PGKEREFVAAINWGGGWTYY | A14P, |
| | | ADSVKGRFTISRDNAKNTLY | D16G |
| | | LQMNSLRPEDTALYYCAASS | S23A, |
| | | DYAGGNPTGYPYWGQGTLVT | M43K, |
| | | VSS | Q64K, |
| | | | K83R, |
| | | | G85E, |
| | | | G89L) |
| ALB00622/ | 43 | EVQLVESGGGVVQPGGSLRL | HSA006A06 |
| T028501805 | | SCAASGFTFRSFGMSWVRQA | (L11V, |
| | | PGKGPEWVSSISGSGSDTLY | N16G, |
| | | ADSVKGRFTISRDNSKNTLY | E44G, |
| | | LQMNSLRPEDTALYYCTIGG | A74S, |
| | | SLSRSSQGTLVTVSS | T76N, |
| | | | K83R, |
| | | | V89L, |
| | | | Q108L) |
| A016600058 | 68 | DVQLVESGGGVVQPGGSLRL | TNF006C11 |
| | | SCTASGFTFSTADMGWFRQA | (E1D, |
| | | PGKGREFVARISGIDGTTYY | L11V, |
| | | DEPVKGRFTISRDNSKNTVY | A14P, |
| | | LQMNSLRPEDTALYYCRSPR | Q27F, |
| | | YADQWSAYDYWGQGTLVTVS | S29F, |
| | | SA | P40A, |
| | | | K73N, |
| | | | A74S, |
| | | | Q75K, |
| | | | D82aN, |
| | | | K83R, |
| | | | V89L, |
| | | | Q108L)-A |
| A044301152 | 250 | EVQLVESGGGVVQPGGSLRL | T0347015C01 |
| | | SCAASGFTFEDYAIGWFRQA | (L11V, |
| | | PGKEREEVSCIRTYDEQTYY | A14P, |
| | | ADSVKGRFTISRDNAKNTVS | D30E, |
| | | LQMNSLRPEDTALYYCAAGS | G55E, |
| | | YYACAYYSRPDPSEGHVDLD | N56Q, |
| | | YWGQGTLVTVSS | I60A, |
| | | | S71R, |
| | | | Y79S, |
| | | | K83R, |
| | | | A89L, |
| | | | K100bY, |
| | | | N100kG, |
| | | | M100oL, |
| | | | K105Q) |
| T042500044 | 225/ | EVQLVESGGGLVQPGGSLRL | A0095013F07 |
| | 195 | SCAASGLTFSTNPMYWYRQA | (A14P, |
| | | PGKQRELVASISSRGITNYA | N27L, |
| | | DSVKGRFTISRDNSKNTVYL | E41P, |
| | | QMNSLRPEDTAVYYCRLASL | R64K, |
| | | SSGTVYWGQGTLVTVSS | S68T, |
| | | | T74S, |
| | | | D76N, |
| | | | K83R, |
| | | | K105Q) |
| TPP-134228 | 226/ | QVQLEESGGGSVQTGGSLRL | EGFR007D12 |
| | 173 | TCAASGRTSRSYGMGWFRQA | (K3Q) |
| | | PGKEREFVSGISWRGDSTGY | |
| | | ADSVKGRFTISRDNAKNTVD | |
| | | LQMNSLKPEDTAIYYCAAAA | |
| | | GSAWYGTLYEYDYWGQGTQV | |
| | | TVSS | |
| T042500096 | 227/ | EVQLVESGGGVVQPGGSLRL | RSV007B02 |
| | 219 | SCAASGRTFSSYAMGWFRQA | (L11V, |
| | | PGKEREFVAAISWSDGSTYY | A14P, |
| | | ADSVKGRFTISRDNAKNTVY | D16G, |
| | | LQMNSLRPEDTALYYCAADL | K83R, |
| | | TSTNPGSYIYIWAYDYWGQG | V89L, |
| | | TLVTVSS | Q108L) |

### Cargos

As described in detail above, the molecule of the present technology comprises at least one ISVD which comprises at least one, preferably at least two, attachment point(s) or conjugation site(s), which is (are) primary amine(s) in the side chain of lysine(s) located at solvent accessible positions, or at the C-terminus of the ISVD, or the N-terminal primary amine, suitable for attachment of different molecules, including proteins, peptides, toxic payloads, nucleic acids, glycans, radio-isotopes, PEG, etc. and combinations thereof, see below for further examples of suitable cargos. In the context of the present technology, a “cargo” is any molecule which is/may be attached or conjugated to the ISVD through the attachment point(s) or conjugation site(s) present therein. Hence, a “cargo”, in the context of the present technology, may be any molecule, including proteins, peptides, toxic payloads, nucleic acids (such as DNA, RNA, siRNA, RNAi, etc.), glycans, radio-isotopes, PEG, biotin, etc. and combinations thereof, see below for specific examples.

Hence, the molecule of the present technology may comprise at least one cargo as defined herein conjugated to one of the attachment points or conjugation sites present in the at least one ISVD.

In a preferred embodiment, the at least one cargo which may be attached to the conjugation site(s) of the at least one ISVD of the present technology is an ISVD as described herein (also referred to in the present description as “cargo ISVD”). In this context, the cargo ISVD may preferably specifically bind to one or more proteins in the human body, such as human proteins and/or may also specifically bind other proteins present in the human body (e.g., viral or bacterial proteins which are in the human body).

In a preferred embodiment, the molecule of the present technology comprises at least one ISVD and at least one cargo attached to one of the conjugation sites or attachment points present in the ISVD, preferably to a conjugation site or attachment point which is the side chain of a Lys preferably located at a solvent-accessible position of the ISVD, as described above, preferably wherein the at least one cargo is also an ISVD.

In another embodiment, the at least one cargo which may be attached to the conjugation site(s) of the at least one ISVD of the present technology is a group, residue, moiety or binding unit which provides the ISVD (and/or molecule) of the present technology with increased (in vivo) half-life compared to the corresponding ISVD/molecule without said one or more other groups, residues, moieties or binding units.

The cargos are attached (or “anchored”, “conjugated”, “linked”) to the at least one ISVD via the at least one conjugation site, as described above. The cargo(s) and the at least one ISVD may be directly linked to each other (as for example described in WO 1999/23221) and/or may be linked to each other via one or more suitable linkers, or any combination thereof. Suitable linkers for use in the molecule of the technology will be clear to the skilled person, and may generally be any linker used in the art to link amino acid sequences or any other molecule comprised in the cargo. Preferably, said linker is suitable for use in constructing proteins or polypeptides that are intended for pharmaceutical use. Some particularly preferred linkers include the linkers that are used in the art to link antibody fragments or antibody domains. These include the linkers mentioned in the publication cited above, as well as for example linkers that are used in the art to construct diabodies or ScFv fragments (in this respect, however, it should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the molecule of the technology; this can be tuned depending on the specific applications, e.g., on the number and nature of the cargos to be attached to the ISVD, on the specific position and number of attachment points or conjugation sites and on the nature of the linker. As also shown in the Examples, the skilled person will be able to select an appropriate linker for a certain application).

For example, a linker may be a suitable amino acid or amino acid sequence, and in particular amino acid sequences of between 1 and 50, preferably between 1 and 30, such as between 1 and 10 amino acid residues. Some preferred examples of such amino acid sequences include Gly-Ser linkers, for example of the type (GlyxSery) z, such as (for example (Gly₄Ser)₃ or (Gly₃Ser₂)₃, as described in WO 1999/42077 and the GS30, GS15, GS9 and GS7 linkers described in the applications by Ablynx mentioned herein (see for example WO 2006/040153 and WO 2006/122825), as well as hinge-like regions, such as the hinge regions of naturally occurring heavy chain antibodies or similar sequences (such as described in WO 1994/04678). Some linkers are depicted in Table A-1 below. For instance, a preferred linker which may be used in the molecule of the present technology is depicted in SEQ ID NO.: 113 (15GS). Some other particularly preferred linkers are poly-alanine (such as AAA), as well as the linkers GS30 (SEQ ID NO: 85 in WO 2006/122825) and GS9 (SEQ ID NO: 84 in WO 06/122825).

Polyethylene glycol (PEG), in any of the variants described below, may also be used as a linker in the molecule of the present technology. Other suitable linkers for use in the molecule of the present technology are described, e.g., in Kjeldsen T. et al. (“Dually reactive long recombinant linkers for bioconjugations as an alternative to PEG”, ACS Omega, 2020, 5:19827-19833). As described therein polar protein sequences with PEG-like properties, sometimes called “recombinant PEG”, have in recent years been described by Alvarez (“Improving protein pharmacokinetics by genetic fusion to simple amino acid sequences”, J. Biol. Chem., 2004, 279:3375-3381), Amumix (mixed sequences of GEDSTAP residues, termed “ELNN polypeptides”, see, e.g., US 2014/0301974 A1), XL-protein (PAS repeats), Novo Nordisk (GQAP-like repeats), SOBI and others (see, e.g., Table A-1 below).

As used herein, the terms “ELNN polypeptides” and “ELNNs” are synonymous and refer to extended length polypeptides comprising non-naturally occurring, substantially non-repetitive sequences (e.g., polypeptide motifs) that are composed mainly of small hydrophilic amino acids, with the sequence having a low degree or no secondary or tertiary structure under physiologic conditions. ELNN polypeptides include unstructured hydrophilic polypeptides comprising repeating motifs of 6 natural amino acids (G, A, P, E, S, and/or T). In some embodiments, an ELNN polypeptide comprises multiple motifs of 6 natural amino acids (G, A, P, E, S, T), wherein the motifs are the same or comprise a combination of different motifs. In some embodiments, ELNN polypeptides can confer certain desirable pharmacokinetic, physicochemical, and pharmaceutical properties when linked to proteins (e.g., when linked to the ISVD of the present technology). Such desirable properties may include but are not limited to enhanced pharmacokinetic parameters and solubility characteristics, as well as improved therapeutic index. ELNN polypeptides are known in the art, and non-limiting descriptions relating to and examples of ELNN polypeptides known as XTEN® polypeptides are available in Schellenberger et al., (2009), Nat Biotechnol 27 (12): 1186-90; Brandl et al., (2020), Journal of Controlled Release 327:186-197; and Radon et al., (2021), Advanced Functional Materials 31, 2101633 (pages 1-33), the entire contents of each of which are incorporated herein by reference.

Ravtansine/soravtansine (N2′-Deacetyl-N2′-(4-mercapto-4-methyl-1-oxopentyl)-maytansine or DM4, CAS Registry Number: 796073-69-3) is a maytansinoid connected via a cleavable chemical linker to the targeting mAb which may be used as a cytotoxic component of, e.g., antibody-drug conjugates. This cleavable linker is also suitable for being used in the molecule of the present technology.

It is encompassed within the scope of the technology that the length, the degree of flexibility and/or other properties of the linker(s) used may have some influence on the properties of the molecule of the technology. Based on the disclosure herein and the disclosure of other publications, such as, for example, WO 2017/089618, the skilled person will be able to determine the optimal linker(s) for use in the specific molecule of the technology, optionally after some limited routine experiments.

Further suitable linkers for use in the molecule of the technology are, e.g., cleavable linkers, i.e., linkers which have a trigger in its structure that can be efficiently cleaved. For instance, Su, Z. et al. (“Antibody-drug conjugates: Recent advances in linker chemistry”, Acta Pharmaceutica Sinica B, 2021, 11 (12): 3889-3907) reviews linkers that may be comprised in antibody-drug conjugates and which may also be used in the molecule of the present technology. For example, suitable linkers for use in the molecule of the present technology are APN-maleimide linker (3-(4-(2,5-dioxo-2,5-dihydro-1H-pyrrol-1-yl)phenyl) propiolonitrile, MAPN) or bis-maleimido-PEG3 (BM (PEG)₃) linker (BM (PEG)₃ (1,11-bismaleimido-triethyleneglycol)).

When two or more linkers are used in the molecule of the technology, these linkers may be the same or different. Again, based on the disclosure herein, the skilled person will be able to determine the optimal linkers for use in the specific molecule of the technology, optionally after some limited routine experiments.

### (In Vivo) Half-Life Extending Moieties

As described above, the molecule of the present technology may comprise one or more other groups, residues, moieties or binding units, optionally linked via one or more linkers, such as peptide linkers, as defined above, in which said one or more other groups, residues, moieties or binding units provide the molecule of the present technology with increased (in vivo) half-life, compared to the corresponding molecule without said one or more other groups, residues, moieties or binding units (“(in vivo) half-life extending moiety”, or “half-life extending (HLE) moiety”). The HLE moiety is a cargo as described above when it is attached or conjugated to the at least one attachment point or conjugation site comprised in the ISVD of the present technology, which is (are) primary amine(s) in the side chain of lysine(s) located at solvent accessible positions, or at the C-terminus, or the N-terminal primary amine.

The term “half-life” as used here can generally be defined as described in paragraph o) on page 57 of WO 2008/020079 and as mentioned therein refers to the time taken for the serum concentration of the compound or polypeptide to be reduced by 50%, in vivo, for example due to degradation of the sequence or compound and/or clearance or sequestration of the sequence or compound by natural mechanisms. The in vivo half-life of the ISVD and/or molecule of the technology can be determined in any manner known per se, such as by pharmacokinetic analysis. Suitable techniques will be clear to the person skilled in the art, and may for example generally be as described in paragraph o) on page 57 of WO 2008/020079. As also mentioned in paragraph o) on page 57 of WO 2008/020079, the half-life can be expressed using parameters such as the t_{1/2}-alpha, t_{1/2}-beta and the area under the curve (AUC). In this respect it should be noted that the term “half-life” as used herein in particular refers to the t_{1/2}-beta or terminal half-life (in which the t_{1/2}-alpha and/or the AUC or both may be kept out of considerations). Reference is for example made to the standard handbooks, such as Kenneth, A et al: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the t_{1/2}-beta, either with or without an increase in the t_{1/2}-alpha and/or the AUC or both.

(In vivo) half-life can be extended by an increase in the hydrodynamic radius (size) or by a decrease in the molecule's clearance. For instance, (in vivo) half-life extending moieties such as polyethylene glycol or ELNN polypeptides increase the size of the molecules to which they are attached, therefore bypassing renal clearance, and thus increasing the half-life of those molecules. Other (in vivo) half-life extending moieties such as binding units that can bind to, e.g., serum albumin, increase the half-life of the molecules to which they are attached by binding, e.g., to serum albumin. Albumin is the most abundant plasma protein, is highly soluble, very stable and has an extraordinarily long circulatory half-life as a direct result of its size and interaction with the FcRn mediated recycling pathway, see, e.g., Sleep D. et al., “Albumin as a versatile platform for drug half-life extension”, Biochim Biophys Acta, 2013, 1830 (12): 5526-34.

The type of groups, residues, moieties or binding units is not generally restricted and may for example be chosen from the group consisting of a polyethylene glycol (PEG) molecule, ELNN polypeptides or fragments thereof, as described above, serum proteins or fragments thereof, binding units that can bind to serum proteins, an Fc portion, and small proteins or peptides that can bind to serum proteins.

More specifically, said one or more other groups, residues, moieties or binding units that provide the molecule of the present technology with increased half-life can be chosen from the group consisting of a polyethylene glycol (PEG) molecule, ELNN polypeptides or fragments thereof, binding units that can bind to serum albumin, such as human serum albumin, or a serum immunoglobulin, such as IgG, or Fc fusions which might provide extra functionalities in vivo such as HLE via FcRn, immune effector functions via Fcγ receptors. In one embodiment, said one or more other groups, residues, moieties or binding units that provide the molecule of the present technology with increased half-life is a binding unit that can bind to human serum albumin. In one embodiment, the binding unit is an ISVD.

For example, WO 2004/041865 describes ISVDs binding to serum albumin (and in particular against human serum albumin) that can be linked or attached to other proteins (such as one or more ISVDs of the present technology) in order to increase the half-life of the molecule of the present technology.

The international application WO 2006/122787, the content of which is herein incorporated by reference, describes a number of ISVDs against (human) serum albumin. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of therapeutic proteins and polypeptides, and other entities or moieties, such as the molecule of the present technology.

Moreover, WO 2012/175400, the content of which is herein incorporated by reference, describes a further improved version of Alb-1, called Alb-23.

In one embodiment, the molecule of the present technology further comprises a serum albumin binding moiety selected from Alb-1, Alb-3, Alb-4, Alb-5, Alb-6, Alb-7, Alb-8, Alb-9, Alb-10 (described in WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO 2015/173325, WO 2017/080850, WO 2017/085172, WO 2018/104444, WO 2018/134235, and WO 2018/134234, the content of which is herein incorporated by reference.

In one embodiment, said one or more other groups, residues, moieties or binding units that provide the molecule with increased half-life is a peptide that can bind to human serum albumin.

In particular the “serum-albumin binding polypeptide or binding domain” may be any suitable serum-albumin binding peptide capable of increasing the half-life (preferably T1/2ß, as defined above) of the molecule (compared to the same molecule without the serum-albumin binding peptide or binding domain).

Specifically, the polypeptide sequence suitable for extending serum half-life is a polypeptide sequence capable of binding to a serum protein with a long serum half-life, such as serum albumin, transferrin, IgG, etc, in particular serum albumin.

Polypeptide sequences capable of binding to serum albumin have previously been described and may in particular be serum albumin binding peptides as described in WO 2008/068280 (and in particular WO 2009/127691 and WO 2011/095545), the content of which is herewith incorporated by reference.

In one embodiment, said one or more other groups, residues, moieties or binding units that provide the molecule with increased half-life is straight or branched chain poly (ethylene glycol) (PEG) or derivatives thereof (such as methoxypoly(ethylene glycol) or mPEG), which increase the hydrodynamic radius of the molecule, thus exceeding the renal clearance and hence rendering the molecule with tuneable half-life extension). Generally, any suitable form of PEGylation can be used, such as the PEGylation used in the art for antibodies and antibody fragments (including but not limited to domain antibodies and scFv's); reference is made, for example, to: Chapman, Nat. Biotechnol., 54, 531-545 (2002); Veronese and Harris, Adv. Drug Deliv. Rev. 54, 453-456 (2003); Harris and Chess, Nat. Rev. Drug. Discov. 2 (2003); WO 04/060965; and U.S. Pat. No. 6,875,841. Various reagents for PEGylation of polypeptides are also commercially available, for example from Nektar Therapeutics, USA, or NOF Corporation,

Japan, such as the Sunbright® EA Series, SH Series, MA Series, CA Series, and ME Series, such as Sunbright® ME-100MA, Sunbright® ME-200MA, and Sunbright® ME-400MA.

After covalent attachment of PEG, molecules can have prolonged blood circulation half-lives, improved drug solubility and stability, and reduced immunogenicity (Swierczewskaa M., et al., “What is the future of PEGylated therapies?”, Expert Opin Emerg Drugs. 2015; 20 (4): 531-536).

The PEG may be linear or branched and have a molecular weight from about 1 to about 40 kDa, such as from about 1 to about 30 kDa, or from about 1 to about 20 kDa, or from about 1 to about 10k Da, preferably from about 2 to about 7 kDa, more preferably from about 4 to about 6k Da and even more preferably of about 5 kDa. The smaller PEG size should enable renal clearance of the PEG moieties, thus bypassing the disadvantages of standard used large 40-60 kDa PEG. In one embodiment, the ISVD of the present technology comprises more than one PEG molecules as described above. For instance, the ISVD of the present technology may comprise 2, 3, 4, 5, 6, 7, 8 or more PEG molecules, such as from 4 to 8 PEG molecules, such as from 5 to 7 PEG molecules, such as 6 PEG molecules. In one embodiment, the ISVD of the present technology comprises 6 molecules of linear 5 kDa PEG. Suitable PEG-groups and methods for attaching them to the ISVD of the present technology will be clear to the skilled person.

As mentioned, other means of increasing the half-life of the molecule of the technology (such as the presence of linear or branched 40-60 kDa PEGylation fused to human albumin or a suitable fragment thereof), although less preferred, are also included in the scope of the technology.

Generally, when the ISVD and/or molecule of the present technology has increased half-life (e.g. through the presence of a half-life increasing ISVD, PEG moieties or any other suitable way of increasing half-life, as described above), the resulting ISVD and/or molecule of the technology preferably has a half-life (as defined herein) that is at least 2 times, preferably at least 5 times, for example at least 10 times or more, such at least 20 times, or at least 50 times, or at least 100 times, or at least 150 times, or at least 200 times, or at least 300 times, or at least 400 times, or at least 500 times, greater than the half-life of the ISVD and/or molecule of the technology without the half-life increasing group, residue, moiety or binding unit (as measured in either in man and/or a suitable animal model, such as mouse or cynomolgus monkey). In particular, the ISVD and/or molecule of the technology may have a half-life (as defined herein) in human subjects of at least 1 day, such as at least 3 days, or at least 7 days, such as at least 10 days, or at least 15 days, or at least 20 days. The skilled person is able to select the HLE moiety based on the desired half-life of the ISVD and/or molecule of the present technology. For certain applications, however, it may be desirable that the ISVD and/or molecule of the technology has shorter half-life (e.g., radio imaging/therapy in a theranostic setting).

For instance, the ISVD in this embodiment may be an ISVD-derived ISVD as defined in any one of SEQ ID NO.: 1-78, 173-250, or a polypeptide which has at least 80% identity with a polypeptide as defined in any one of SEQ ID NO.: 1-78, 173-250, preferably at least 85%, more preferably at least 90%, or at least 95%, or at least 99% identity with a polypeptide as defined in any one of SEQ ID NO.: 1-78, 173-250.

For instance, the ISVD in this embodiment may be an ISVD-derived ISVD as defined in any one of SEQ ID NO.: 1, 3-26, 28-43, 45-75, 78 and 173-250, or a polypeptide which has at least 80% identity with a polypeptide as defined in any one of SEQ ID NO.: 1, 3-26, 28-43, 45-75, 78 and 173-250, preferably at least 85%, more preferably at least 90%, or at least 95%, or at least 99% identity with a polypeptide as defined in any one of SEQ ID NO.: 1, 3-26, 28-43, 45-75, 78 and 173-250.

Hence, the present technology further provides a molecule comprising or, alternatively consisting of, at least one ISVD as described herein and at least one (in vivo) half-life extending moiety as described herein.

For instance, the molecule of the present technology may comprise (i) an ISVD comprising or, alternatively, consisting of SEQ ID NO.: 1-78, 173-250, or a polypeptide which has at least 80% identity with a polypeptide as defined in any one of SEQ ID NO.: 1-78, 173-250, preferably at least 85%, more preferably at least 90%, or at least 95%, or at least 99% identity with a polypeptide as defined in any one of SEQ ID NO.: 1-78, 173-250, and (ii) a half-life extending moiety as described herein, such as a serum albumin binding ISVD, and/or a PEG molecule, and/or a ELNN polypeptide or a fragment thereof.

For instance, the molecule of the present technology may comprise (i) an ISVD comprising or, alternatively, consisting of SEQ ID NO.: 1, 3-26, 28-43, 45-75, 78 and 173-250, or a polypeptide which has at least 80% identity with a polypeptide as defined in any one of SEQ ID NO.: 1, 3-26, 28-43, 45-75, 78 and 173-250, preferably at least 85%, more preferably at least 90%, or at least 95%, or at least 99% identity with a polypeptide as defined in any one of SEQ ID NO.: 1, 3-26, 28-43, 45-75, 78 and 173-250, and (ii) a half-life extending moiety as described herein, such as a serum albumin binding ISVD, and/or a PEG molecule, and/or a ELNN polypeptide or a fragment thereof.

The half-life extending moiety may be covalently attached to the conjugation site on the ISVD either directly or by means of a linker, such as a linker selected from the linkers depicted in Table A-1. For instance, the half-life extending moiety may be covalently attached to the ISVD by means of a linker, such as a 15GS linker (SEQ ID NO.: 113).

### Targeting Moieties

As described above, the ISVD may have attached or conjugated, via its one or more conjugation sites or attachment points which is (are) primary amine(s) in the side chain of lysine(s) located at solvent accessible positions, or at the C-terminus, or the N-terminal primary amine, one or more groups, residues, moieties or binding units, optionally attached via one or more linkers, in which said one or more other groups, residues, moieties or binding units target the molecule of the present technology to target molecules on cells, organs or tissues (“targeting moiety”). For instance, the molecule of the present technology may comprise one, two, three, four, five, six, seven, eight, nine, ten or more targeting moieties attached or conjugated to the at least one ISVD.

A targeting moiety, as defined herein, is any group, residue, moiety, or binding unit which is capable of being directed through its binding to a target. An amino acid sequence (such as an ISVD, an antibody, antigen-binding domains or fragments such as V_{HH} domains or V_{H}/V_{L} domains, or generally an antigen binding protein or polypeptide or a fragment thereof) that “(specifically) binds”, that “can (specifically) bind to”, that “has affinity for” and/or that “has specificity for” a specific antigenic determinant, epitope, antigen or protein, or for a specific non-protein molecule, such as nucleic acids (such as DNA or RNA) or glycans (or for at least one part, fragment or epitope thereof) is said to be “against” or “directed against” said antigenic determinant, epitope, antigen, protein or non-protein molecule. Specific binding of an antigen-binding protein to an antigen or antigenic determinant can be determined in any suitable manner known per se, including, for example, Scatchard analysis and/or competitive binding assays, such as radio-immunoassays (RIA), enzyme immunoassays (EIA) and sandwich competition assays, and the different variants thereof known per se in the art; as well as the other techniques mentioned herein.

### Therapeutic Moieties or Precursors Therefrom

As described above, the ISVD of the present technology may have attached or conjugated, via its one or more conjugation sites or attachment points, which is (are) primary amine(s) in the side chain of lysine(s) located at solvent accessible positions, or at the C-terminus, or the N-terminal primary amine, to one or more other groups, residues, moieties or binding units, optionally linked via one or more peptidic linkers, in which said one or more other groups, residues, moieties or binding units are capable of exerting a therapeutic activity in the animal or human body (“therapeutic moiety or precursor therefrom”). For instance, the molecule of the present technology may comprise one, two, three, four, five, six, seven, eight, nine, ten or more therapeutic moieties or precursors therefrom attached or conjugated to the at least one ISVD.

A therapeutic moiety, as defined herein, is any group, residue, moiety, or binding unit which is capable of exerting a therapeutic activity in the animal and/or human body. The therapeutic moiety may also be in the form of a precursor, which then gets activated to exert its therapeutic activity. For instance, a therapeutic moiety according to the present technology may be any therapeutic agent such as a drug, protein, peptide, gene, compound or any other pharmaceutically active ingredient which may be used for the treatment and/or prevention of a certain disease condition. For instance, a therapeutic moiety may be a therapeutic antibody, or a therapeutic ISVD.

### Imaging Moieties

As described above, the ISVD may have attached or conjugated, via its one or more conjugation sites or attachment points, which is (are) primary amine(s) in the side chain of lysine(s) located at solvent accessible positions, or at the C-terminus, or the N-terminal primary amine, one or more other groups, residues, moieties or binding units, optionally linked via one or more peptidic linkers, wherein said one or more other groups, residues, moieties or binding units are used for imaging purposes (“imaging moiety”). For instance, the molecule of the present technology may comprise one, two, three, four, five, six, seven, eight, nine, ten or more imaging moieties attached or conjugated to the at least one ISVD.

Examples of imaging moieties are provided in Agdeppa E D, Spilker M E. A review of imaging agent development. AAPS J. 2009 June;11 (2): 286-99. For instance, the imaging moiety present in the molecule of the present technology may be suitable for radiotherapy and for radio/fluorescence-guided cancer surgery. For instance, the imaging moiety may comprise radioactive isotopes that can be used for diagnostic and therapeutic proposes. For instance, the imaging moiety may be a contrast agent. For instance, the imaging moiety may be a non-radioactive medical isotope.

### Toxic Moieties or Drugs

As described above, the ISVD may have attached or conjugated, via its one or more conjugation sites or attachment points, which is (are) primary amine(s) in the side chain of lysine(s) located at solvent accessible positions, or at the C-terminus, or the N-terminal primary amine, one or more other groups, residues, moieties or binding units, optionally linked via one or more (e.g., cleavable or non-cleavable, peptidic or non-peptidic) linkers, wherein said one or more other groups, residues, moieties or binding units are able to impart certain toxicity to cells and/or tissues (“toxic moiety” or “drug”). For instance, the molecule of the present technology may comprise one, two, three, four, five, six, seven, eight, nine, ten or more toxic moieties attached or conjugated to the at least one ISVD.

A toxic moiety which may be attached or conjugated to the ISVD may belong to the “tubulin inhibitor” family (e.g., maytansinoids, auristatins, taxol derivates) or to the “DNA-modifying agents” family (e.g., calicheamicins, duocarymycins). They can also be antibiotics or enzymes. For a review, see Criscitiello C. et al., “Antibody-drug conjugates in solid tumors: a look into novel targets”, Hematol Oncol, 2021, 14:20.

### Nucleic Acids

The ISVD may also have attached or conjugated, via its one or more conjugation sites or attachment points, which is (are) primary amine(s) in the side chain of lysine(s) located at solvent accessible positions, or at the C-terminus, or the N-terminal primary amine, one or more nucleic acid such as RNA or DNA. For instance, the molecule of the present technology may comprise one, two, three, four, five, six, seven, eight, nine, ten or more nucleic acid molecules attached or conjugated to the at least one ISVD.

### Vitamins

Vitamins are also suitable cargos to be attached to the conjugation sites or attachment points, which is (are) primary amine(s) in the side chain of lysine(s) located at solvent accessible positions, or at the C-terminus, or the N-terminal primary amine, present in the at least one

ISVD comprised in the molecule of the present technology. Non-limiting examples of vitamins are folate (folic acid), biotin, vitamin C, etc.

### Nucleic Acid Molecules

The present technology also provides a nucleic acid molecule encoding the ISVD and/or the molecule (or part of the molecule) of the present technology.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable (non-human) hosts or host cells for production purposes will be clear to the skilled person, and may for example be any suitable fungal, prokaryotic or eukaryotic cell or cell line or any suitable fungal, prokaryotic or eukaryotic organism. A (non-human) host or host cell comprising a nucleic acid encoding the ISVD and/or the molecule (or part of the molecule) of the present technology is also encompassed by the present technology.

A nucleic acid may be for example DNA, RNA, or a hybrid thereof, and may also comprise (e.g., chemically) modified nucleotides, like PNA. It can be single- or double-stranded. In one embodiment, it is in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly) peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### Vectors

Also provided is a vector comprising the nucleic acid molecule encoding the ISVD and/or the molecule (or part of the molecule) of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

In some embodiments, vectors comprise at least one nucleic acid that is optionally linked to one or more regulatory elements, such as for example one or more suitable promoter(s), enhancer(s), terminator(s), etc.). In one embodiment, the vector is an expression vector, i.e.

a vector suitable for expressing an encoded polypeptide or construct under suitable conditions, e.g. when the vector is introduced into a (e.g. human) cell. DNA-based vectors include the presence of elements for transcription (e.g., a promoter and a polyA signal) and translation (e.g. Kozak sequence).

In one embodiment, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

In one embodiment, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

### Compositions

The present technology also provides a composition comprising the ISVD and/or the molecule of the present technology. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### Host Organisms

The present technology also pertains to host cells or host organisms expressing the ISVD and/or the molecule (or part of the molecule) of the present technology, comprising the nucleic acid encoding the ISVD and/or the molecule (or part of the molecule) of the present technology, and/or the vector comprising the nucleic acid molecule encoding the ISVD and/or the molecule (or part of the molecule) of the present technology.

Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Komagataella phaffii (Pichia pastoris, see Bernauer L., et al. (“Komagataella phaffii as emerging model organism in fundamental research”, Front. Microbiol., 2021, 11:1-16)). In one embodiment, the host is Komagataella phaffii (Pichia pastoris). In another embodiment, the host is Escherichia coli. Of course, cell free systems may also be employed to produce the ISVD and/or the molecule of the present technology, as reviewed, for instance, in Gregorio N E, Levine M Z, Oza J P, “A user's guide to cell-free protein synthesis”, Methods Protoc. 2019,2 (1): 24.

### Methods and Uses of the Molecule

The present technology also provides a method for producing the ISVD and/or the molecule of the present technology. The method may comprise transforming/transfecting a host cell or host organism with a nucleic acid encoding the at least one ISVD and/or the molecule (or part of the molecule), expressing the at least one ISVD and/or the molecule (or part of the molecule) in the host, optionally followed by one or more isolation and/or purification steps. Specifically, the method may comprise:
a) expressing, in a suitable host cell or (non-human) host organism or in another suitable expression system, a nucleic acid sequence encoding the at least one ISVD and/or the molecule (or part of the molecule); optionally followed by:
b) isolating and/or purifying the at least one ISVD and/or the molecule (or part of the molecule).

For instance, the ISVD and/or the molecule (or part of the molecule) of the present technology may be encoded in a nucleic acid together with a Protein A binding building block, so that the the ISVD and/or the molecule (or part of the molecule) can be easily purified with Protein A chromatography after expression. Hence, Protein A chromatography can be employed to purify the ISVD and/or the molecule (or part of the molecule). Further purification steps such as size exclusion chromatography (SEC) or ultrafiltration and/or ion-exchange chromatography may be applied in order to purify the ISVD and/or the molecule (or part of the molecule).

To produce/obtain the at least one ISVD and/or the molecule of the present technology, both in genetic fusion or a single polypeptide, the host cell or host organism or cell free system may generally be kept, maintained and/or cultured under conditions such that the (desired) ISVD and/or molecule of the technology is optimally expressed/produced. Suitable conditions will be clear to the skilled person and will usually depend upon the host cell/host organism or cell free system used, as well as on the regulatory elements that control the expression of the protein of the technology.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Komagataella phaffii (Pichia pastoris). In one embodiment, the host is Komagataella phaffii (Pichia pastoris). In another embodiment, the host is Escherichia coli.

Hence, the at least one ISVD and/or the molecule (or part of the molecule) of the present technology can be encoded in a nucleic acid molecule, optionally as part of an expression vector, and expressed and produced recombinantly, as described above.

In one embodiment, the molecule of the present technology comprises more than one ISVD as defined above. For instance, the molecule of the present technology may comprise 2, 3 or more ISVDs as defined above. The more than one ISVD of the present technology can be encoded in a single nucleic acid molecule, optionally as part of an expression vector, and expressed and produced recombinantly, as described above.

In addition to the at least one ISVD, the molecule of the present technology may further comprise one or more other groups, residues, moieties or binding units (cargos) in which said one or more other groups, residues, moieties or binding units provide the molecule with several functionalities, such as binding specificity (e.g., by the presence of a targeting moiety in the molecule of the present technology), increased (in vivo) half-life extension (e.g., by the presence of half-life extending moiety in the molecule of the present technology), therapeutic properties (e.g., by the presence of a pharmaceutically active moiety in the molecule of the present technology), etc.

The one or more ISVDs and/or the at least one cargo comprised in the molecule of the present technology may be recombinantly expressed as part of one or more genetic construct(s) and/or may be independently chemically synthesized (e.g., by SPPS). For instance, one or more ISVD(s) may be expressed recombinantly, as part of a single genetic construct, and the one or more cargo(s) may also be expressed as part of another genetic construct. In a further step, the one or more cargo(s) may be attached or conjugated to the at least one ISVD(s) through the conjugation site(s) or attachment point(s), which is (are) primary amine(s) in the side chain of lysine(s) located at solvent accessible positions, or at the C-terminus, or the N-terminal primary amine.

The molecule of the present technology may comprise at least one ISVD and one or more other groups, residues, moieties or binding units, wherein the at least one ISVD and the one or more other groups, residues, moieties or binding units are part of a single genetic construct and expressed recombinantly, i.e., they are expressed recombinantly as a single polypeptide. Further groups, residues, moieties or binding units may then be attached or conjugated to one or more conjugation site(s) or attachment point(s) present in the ISVD(s), which is (are) primary amine(s) in the side chain of lysine(s) located at solvent accessible positions, or at the C-terminus, or the N-terminal primary amine.

For instance, the molecule of the present technology may comprise two or more ISVDs which may be part of a genetic construct and recombinantly expressed.

For instance, the molecule of the present technology may comprise one or more ISVD and one or more other groups, residues, moieties or binding units in which said one or more other groups, residues, moieties or binding units provide the molecule with several functionalities, such as binding specificity, increased (in vivo) half-life extension, therapeutic properties etc. In this case, the one or more ISVD and the one or more other groups, residues, moieties or binding units may be part of a single genetic construct and recombinantly expressed as a single polypeptide.

For instance, the molecule of the present technology may comprise one ISVD and one half-life extension moiety, wherein the half-life extension moiety and ISVD may be part of a genetic construct and expressed recombinantly as a single polypeptide.

Hence, the molecule of the present technology may comprise or consist of more than one ISVD(s), which may be part of a genetic construct and expressed recombinantly as a single polypeptide. One or more cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more ISVD(s) and one or more half-life extending moieties, as described above, they may be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more ISVD(s) and one or more targeting moieties, as described above, and they may all be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more

ISVD(s) and one or more therapeutic moieties, as described above, and they may all be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more ISVD(s) and one or more targeting and/or therapeutic moiety, as described above, and they may all be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more

ISVD(s), one or more half-life extending moiety and one or more targeting moiety, as described above, and they may all be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more ISVD(s), one or more half-life extending moiety and one or more therapeutic moiety, as described above, and they may all be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

In another embodiment, the molecule of the present technology may comprise one or more ISVD(s), one or more half-life extending moiety and/or one or more targeting and/or therapeutic moiety, as described above, and they may all be part of a genetic construct and expressed recombinantly as a single polypeptide. One or more further cargos, as defined above, may then be attached or conjugated to one or more attachment points or conjugation sites present in the ISVD(s), which is (are) primary amine(s) in the side chain of lysine(s) located at solvent accessible positions, or at the C-terminus, or the N-terminal primary amine.

Alternatively, the at least one ISVD and/or molecule (or part of the molecule) of the present technology can be produced synthetically, e.g., using solid-phase peptide synthesis (SPPS), see, e.g., Jaradat, D. M. M., Thirteen decades of peptide synthesis: key developments in solid phase peptide synthesis and amide bond formation utilized in peptide ligation, Amino Acids 50, 39-68 (2018).

As it will be evident to the skilled reader, if the molecule of the present technology comprises one or more ISVDs and, optionally, one or more other groups, residues, moieties or binding units, as defined above, part or the whole molecule may be encoded in a nucleic acid molecule, optionally as part of an expression vector, as defined above, and part or the whole molecule may be produced synthetically.

Once the one or more ISVDs and, optionally, the one or more other groups, residues, moieties or binding units, as defined above, are produced, the cargos may be attached to the at least one ISVD via the attachment points or conjugation sites (preferably engineered conjugation sites or attachment points), as described above. For instance, the at least one ISVD may be expressed recombinantly, as described above, and the cargo(s) conjugated to it via the at least one attachment point or conjugation site, thus rendering the molecule of the present technology. For instance, the at least one ISVD may be produced synthetically, e.g., using

SPPS, as described above, and the cargo(s) conjugated to it via the at least one attachment point or conjugation site, thus rendering the molecule of the present technology. For instance, the at least one ISVD and one or more further moieties, such as HLE moieties, may be encoded in an expression vector and be expressed recombinantly, as described above, and the cargo(s) conjugated to the ISVD via the at least one attachment point or conjugation site, thus rendering the molecule of the present technology. The at least one ISVD and one or more further moieties, such as HLE moieties may be linked through a linker, as described in detail above.

For instance, the at least one ISVD may be expressed recombinantly (alone or together with, e.g., at least some of the half-life extension moieties), as described above, and the cargo(s) conjugated to it via the at least one attachment point or conjugation site. The cargo may be attached or conjugated to the ISVD (directly or by means of a linker) by reaction of a group present in the cargo/linker (e.g., isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, or fluorophenyl esters) and the one or more primary amines present in the ISVD as conjugation sites (if any).

The skilled person is familiar with groups, residues, or moieties able to provide therapeutic properties to the molecule of the present technology, such as pharmaceutically active moieties. The skilled person is also familiar with groups, residues or moieties able to provide specific targeting of the molecule of the technology to desired organs/tissues/cells in the human or animal body, such as targeting moieties.

For instance, at least some of the half-life extension moieties, targeting moieties, therapeutic moieties or precursors therefrom described above in the “Cargos” section may be incorporated in the molecule of the present technology as part of a genetic construct, expressed recombinantly, possibly together with the at least one ISVD, as described in detail above. Hence, at least some of the half-life extension moieties, targeting moieties, therapeutic moieties or precursors therefrom or imaging molecules described above in the “Cargos” section may be incorporated in the molecule of the present technology (i) by attaching or conjugating them to the at least one attachment point or conjugation site present in the ISVD, which is (are) primary amine(s) in the side chain of lysine(s) located at solvent accessible positions, or at the C-terminus, or the N-terminal primary amine, or (ii) by expressing them recombinantly together with the ISVD. Of course, combinations of the above mechanisms are possible; for instance, one or more of the half-life extension moieties, targeting moieties, therapeutic moieties or precursors therefrom described above in the

“Cargos” section may be incorporated in the molecule of the present technology as part of a genetic construct, expressed recombinantly possibly together with the at least one ISVD, and/or one or more of the half-life extension moieties, targeting moieties, therapeutic moieties or precursors therefrom described above in the “Cargos” section may be incorporated in the molecule of the present technology by attaching or conjugating them to the at least one attachment point or conjugation site present in the ISVD. The skilled person will understand and decide how to generate the molecule of the present technology in light of the number of ISVDs and specific moieties and/or cargos that the molecule will incorporate.

If two or more proteins (e.g., one ISVD and one further protein, such as one ISVD, which may be a targeting moiety, which may increase the in vivo half-life of the ISVD and/or molecule of the present technology and/or which may have therapeutic properties) are comprised in the molecule of the present technology, they may be directly linked to each other, and/or may be linked to each other via one or more suitable linkers, or any combination thereof. Suitable linkers have been described above in this description.

As already described, the conjugation of cargos to the attachment points or conjugation sites may be performed directly or via a linker.

In one embodiment, the present technology provides a method for producing the ISVD suitable for site-specific conjugation of the present technology, the method comprising the steps of:
Step a. Providing an ISVD, wherein the ISVD comprises at least one lysine in its amino acid sequence. The lysine is preferably present at a solvent-accessible position, as described herein, and it is preferably not in the CDRs. For instance, the Lys may be present as at positions 43, 64, 75 and/or 83 (according to Kabat). The ISVD provided in step a. above is the so-called ISVD precursor, as defined herein.
Step b. Substituting the at least one lysing by another amino acid. The substitution of the at least one Lys, preferably of all Lys present in the sequence of the ISVD provided in step a. (the ISVD precursor), can be performed by point mutations, as described herein. Preferably, the at least one Lys (preferably all Lys) present in the ISVD precursor, is (are) substituted in step b., by another amino acid selected from glutamine, histidine, arginine, alanine or glutamate, more preferably arginine, to generate an ISVD with no lysines in its sequence.

In addition, in this step b., the first amino acid (position 1 according to Kabat, N-terminal amino acid) of the ISVD precursor may also be substituted by a Gln (Q). More preferably, the first amino acid comprises a pyroglutamate modification.
Step c. Substituting at least one amino acid present at a solvent accessible position, as defined above, by a lysine in the ISVD provided in step b., to generate the ISVD suitable for site-specific conjugation of the present technology. As described above, preferably, the one or more amino acids to be substituted in step c. are selected from the amino acids at positions 5, 7, 10, 12, 13, 19, 43, 44, 45, 61, 64, 70, 71, 73, 75, 83, 85, 105 and/or 108 (Kabat), preferably at positions 19, 43, 45, 64, 71, 73, 75, 83 and/or 105.

Preferably, if not performed in step b., the substitution of the first amino acid (position 1 according to Kabat, N-terminal amino acid) of the ISVD precursor may also be substituted by a Gln (Q). More preferably, the first amino acid comprises a pyroglutamate modification.

The technology further provides method for producing an immunoglobulin single variable domain (ISVD) suitable for lysine site-specific conjugation comprising the steps of:
a. Providing an ISVD with no lysines in its sequence;
b. Substituting at least one amino acid present at a solvent accessible position in the ISVD provided in step a. by a lysine, to generate an ISVD suitable for lysine site-specific conjugation.

The technology further provides method for producing an immunoglobulin single variable domain (ISVD) suitable for lysine site-specific conjugation comprising the steps of:
a. Providing an ISVD with no lysines in its sequence and an N-terminus comprising a pyroglutamate modification (i.e., providing an ISVD without any primary amine in its sequence);
b. Substituting at least one amino acid present at a solvent accessible position in the ISVD provided in step a. by a lysine, to generate an ISVD suitable for lysine site-specific conjugation.

Further, the technology provides a method for producing an immunoglobulin single variable domain (ISVD) without any lysines in its amino acid sequence, comprising the steps of:
a. Providing an ISVD, preferably wherein the ISVD comprises at least one lysine in its amino acid sequence, preferably at a solvent accessible position;
b. Substituting the at least one lysing by another amino acid, preferably selected from glutamine, histidine, arginine, alanine, or glutamate, more preferably by arginine, to generate an ISVD without any lysines in its amino acid sequence.

Further, the technology provides a method for producing an immunoglobulin single variable domain (ISVD) without any lysines in its amino acid sequence, comprising the steps of:
a. Providing an ISVD, preferably wherein the ISVD comprises at least one lysine in its amino acid sequence, preferably at a solvent accessible position, and an N-terminus comprising a pyroglutamate modification;
b. Substituting the at least one lysing by another amino acid, preferably selected from glutamine, histidine, arginine, alanine, or glutamate, more preferably by arginine, to generate an ISVD without any lysines in its amino acid sequence.

As described above, the solvent accessible position are preferably selected from the following positions: 5, 7, 10, 12, 13, 19, 43, 44, 45, 61, 64, 70, 71, 73, 75, 83, 85, 105 and/or 108 (according to Kabat), preferably from the following positions: 19, 43, 45, 64, 71, 73, 75, 83 and/or 105, such as at positions 43 and/or 45.

Hence, with this method, the ISVD of the present technology can be generated. The technology thus provides an ISVD suitable for site-specific conjugation directly produced by the methods of the present technology.

The molecule of the present technology or the composition comprising the molecule of the present technology are useful as a medicament.

Accordingly, the present technology provides the molecule of the present technology or a composition comprising the molecule of the present technology for use as a medicament.

Also provided is the molecule of the present technology or a composition comprising the molecule of the present technology for use in the (prophylactic and/or therapeutic) treatment.

Also provided is the molecule of the present technology or a composition comprising the molecule of the present technology for use in the (prophylactic and/or therapeutic) treatment of an autoimmune/inflammatory disease and/or proliferative diseases/cancer, such as hematological (blood) and solid tumor cancer disease.

Also provided is the molecule of the present technology or a composition comprising the molecule of the present technology for use in the (prophylactic and/or therapeutic) treatment of an infectious disease.

Also provided is the molecule of the present technology or a composition comprising the molecule of the present technology for use as a vaccine. Hence, the present technology provides a vaccine comprising the molecule of the present technology or a composition comprising the molecule of the present technology, optionally further comprising further components such as pharmaceutically acceptable carriers and/or adjuvants.

A “subject” as referred to in the context of the present technology can be any animal. In one embodiment, the subject is a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal. In one embodiment, the subject is a human subject.

Substances, including molecules or compositions may be administered to a subject by any suitable route of administration, for example by enteral (such as oral or rectal) or parenteral (such as epicutaneous, sublingual, buccal, nasal, intratracheal, intra-articular, intradermal, intramuscular, intraperitoneal, intravenous, subcutaneous, transdermal, or transmucosal) administration. In one embodiment, substances are administered by parenteral administration, such as intramuscular, subcutaneous or intradermal, administration.

An effective amount of a molecule as described, or a composition comprising the molecule of the present technology can be administered to a subject in order to provide the intended treatment results.

One or more doses can be administered. If more than one dose is administered, the doses can be administered in suitable intervals in order to maximize the effect of the molecule or composition comprising the same.

### EXAMPLES

### Example 1. Binding Kinetics and Thermal Stability of ISVDs with Substituted Lysine Residues

The framework of an ISVD contains three canonical lysine residues, at positions 43, 64 and 75 (Kabat numbering). Point mutations were introduced in three different ISVDs T028501789, T028501817 and T028501805 each time substituting one of the lysine residues with glutamine, histidine, arginine, alanine, or glutamate. The ISVDs were produced in E. coli, purified using protein A chromatography, and buffer-exchanged to D-PBS.

To evaluate potential effects of the mutations on the thermal stability, a thermal shift assay (TSA) was performed in a 96-well plate on a qPCR machine (LightCycler 48011, Roche). ISVDs were analyzed in the following pH range: 4, 5, 6, 7, 8 and 9. Per well, 5 μL of ISVD sample (0.8 mg/mL in D-PBS) was added to 5 μL of Sypro Orange (40× in MilliQ water; Invitrogen, Cat. No. S6551) and 10 μL of buffer (100 mM phosphate, 100 mM borate, 100 mM citrate and 115 mM NaCl with a pH ranging from 4 to 9). A temperature gradient (37 to 99° C. at a rate of 0.03° C./s) was applied, which induced unfolding of the ISVD, and hence exposure of hydrophobic patches. Binding of Sypro Orange to those hydrophobic patches caused increase in fluorescence intensity, which was measured (Ex/Em=465/580 nm). The inflection point of the first derivative of the fluorescence intensity curve at pH 7 served as a measure of the melting temperature (Tm). The Tm (° C.) values obtained for ISVD variants are given in Table 13.

The affinity (K_{D}) of the lead panel clones to their respective ligands recombinant human CEACAM5 (in-house production) and human serum albumin (Sigma catn. A8763) was determined via SPR on a Biacore 8K+ (Cytiva). The experiment was performed at 25° C. and as running buffer HBS-EP+1× (GE Healthcare, cat #BR-1006-69) was used. The ligands were directly immobilized (amine coupling) on Flow Cell 2 (FC) of a Series S Sensor Chip C M5 for CEACAM5 and C1 for human serum albumin. Flow rate during activation, immobilization and deactivation was 10 μl/min. An affinity determination was set up with a 9-point dilution series (2500-1.64 nM). Affinity determination was performed using a 1:1 binding fit model. Samples were applied to the respective targets in multi-cycle kinetics for 2 minutes, followed by a constant flow of the running buffer for 10 minutes. Between the different injections, the surfaces were regenerated with 10 mM Glycin pH 1.5. The equilibrium dissociation constant K_{D} was calculated as the kd/ka ratio. Results are shown in Table 13.

TABLE 13
Melting temperature and affinity constants of different
ISVDs variants wherein one lysine has been substituted
and comparison with the reference parental precursor ISVD

| | | | | | | | |
|---|---|---|---|---|---|---|---|
| | SEQ ID | mutations | | ΔTm vs. | | | |
| Variant | NO | vs. ref. | Tm | reference | ka (1/Ms) | kd (1/s) | KD (M) |

Precursor ISVD = T028501789

| | | | | | | | |
|---|---|---|---|---|---|---|---|
| T028501794 | 7 | K43A | 77 | −3 | 5.62E+05 | 8.65E−04 | 1.54E−09 |
| T028501790 | 3 | K43E | 76 | −4 | 4.98E+05 | 8.50E−04 | 1.71E−09 |
| T028501793 | 6 | K43H | 77 | −3 | 7.19E+05 | 8.77E−04 | 1.22E−09 |
| T028501791 | 4 | K43Q | 77 | −3 | 6.02E+05 | 8.70E−04 | 1.45E−09 |
| T028501792 | 5 | K43R | 79 | −1 | 7.47E+05 | 9.20E−04 | 1.23E−09 |
| T028501799 | 12 | K64A | 78 | −2 | 3.85E+05 | 9.76E−04 | 2.54E−09 |
| T028501795 | 8 | K64E | 76 | −4 | 2.58E+05 | 9.14E−04 | 3.55E−09 |
| T028501798 | 11 | K64H | 76 | −4 | 4.02E+05 | 9.62E−04 | 2.39E−09 |
| T028501796 | 9 | K64Q | 77 | −3 | 4.15E+05 | 9.29E−04 | 2.24E−09 |
| T028501797 | 10 | K64R | 80 | 0 | 6.75E+05 | 1.07E−03 | 1.59E−09 |
| T028501804 | 17 | K75A | 78 | −2 | 5.86E+05 | 9.17E−04 | 1.56E−09 |
| T028501800 | 13 | K75E | 77 | −3− | 5.29E+05 | 9.11E−04 | 1.72E−09 |
| T028501803 | 16 | K75H | 78 | −2 | 5.74E+05 | 8.40E−04 | 1.46E−09 |
| T028501801 | 14 | K75Q | 78 | −2 | 5.79E+05 | 9.13E−04 | 1.58E−09 |
| T028501802 | 15 | K75R | 80 | 0 | 6.43E+05 | 8.97E−04 | 1.39E−09 |
| T028501789 | 1 | None | 80 | — | 7.16E+05 | 8.85E−04 | 1.24E−09 |

Precursor ISVD = T028501817

| | | | | | | | |
|---|---|---|---|---|---|---|---|
| T028501822 | 32 | K43A | 77 | −2 | 2.25E+05 | 1.98E−03 | 8.77E−09 |
| T028501818 | 28 | K43E | 76 | −3− | 2.15E+05 | 2.26E−03 | 1.05E−08 |
| T028501821 | 31 | K43H | 76 | −3 | 2.12E+05 | 1.93E−03 | 9.11E−09 |
| T028501819 | 29 | K43Q | 77 | −2 | 2.28E+05 | 1.99E−03 | 8.74E−09 |
| T028501820 | 30 | K43R | 79 | 0 | 2.42E+05 | 1.72E−03 | 7.13E−09 |
| T028501827 | 37 | K64A | 78 | −1 | 2.95E+05 | 2.35E−03 | 7.95E−09 |
| T028501823 | 33 | K64E | 76 | −3 | 1.55E+05 | 2.82E−03 | 1.82E−08 |
| T028501826 | 36 | K64H | 76 | −3 | 2.75E+05 | 2.32E−03 | 8.42E−09 |
| T028501824 | 34 | K64Q | 77 | −2 | 2.05E+05 | 2.49E−03 | 1.22E−08 |
| T028501825 | 35 | K64R | 79 | 0 | 3.31E+05 | 2.41E−03 | 7.27E−09 |
| T028501832 | 42 | K75A | 78 | −1 | 3.22E+05 | 2.04E−03 | 6.34E−09 |
| T028501828 | 35 | K75E | 77 | −2 | 2.70E+05 | 2.03E−03 | 7.51E−09 |
| T028501831 | 41 | K75H | 78 | −1 | 2.68E+05 | 2.09E−03 | 7.80E−09 |
| T028501829 | 39 | K75Q | 78 | −1 | 2.91E+05 | 2.08E−03 | 7.14E−09 |
| T028501830 | 40 | K75R | 79 | 0 | 3.44E+05 | 2.07E−03 | 6.01E−09 |
| T028501817 | 26 | None | 79 | — | 2.38E+05 | 2.12E−03 | 8.90E−09 |

Precursor ISVD = T028501805/ALB00622

| | | | | | | | |
|---|---|---|---|---|---|---|---|
| T028501809 | 48 | K43A | 72 | −2 | 7.69E+05 | 8.72E−04 | 1.13E−09 |
| T028501806 | 45 | K43E | 71 | −3 | 8.56E+05 | 1.08E−03 | 1.26E−09 |
| T028501808 | 47 | K43H | 72 | −2 | 7.28E+05 | 1.02E−03 | 1.40E−09 |
| T028501807 | 46 | K43Q | 73 | −1 | 6.77E+05 | 1.19E−03 | 1.76E−09 |
| T028501833 | 56 | K43R | 74 | 0 | 7.03E+05 | 9.06E−04 | 1.29E−09 |
| T028501835 | 58 | K64A | 73 | −1 | 5.24E+05 | 1.08E−03 | 2.06E−09 |
| T028501810 | 49 | K64E | 71 | −3 | 6.44E+05 | 1.24E−03 | 1.93E−09 |
| T028501812 | 51 | K64H | 71 | −3 | 5.90E+05 | 1.02E−03 | 1.74E−09 |
| T028501811 | 50 | K64Q | 72 | −2 | 5.87E+05 | 1.03E−03 | 1.76E−09 |
| T028501834 | 57 | K64R | 74 | 0 | 1.01E+06 | 1.12E−03 | 1.11E−09 |
| T028501816 | 55 | K75A | 73 | −1 | 7.99E+05 | 1.05E−03 | 1.31E−09 |
| T028501813 | 52 | K75E | 72 | −2 | 5.14E+05 | 1.02E−03 | 1.98E−09 |
| T028501836 | 59 | K75H | 72 | −2 | 8.39E+05 | 1.14E−03 | 1.35E−09 |
| T028501814 | 53 | K75Q | 73 | −1 | 8.14E+05 | 1.14E−03 | 1.40E−09 |
| T028501815 | 54 | K75R | 74 | 0 | 8.60E+05 | 1.27E−03 | 1.48E−09 |
| T028501805 | 43 | None | 74 | — | 7.72E+05 | 9.92E−04 | 1.28E−09 |

### Example 2. Binding Kinetics and Thermal Stability of ISVDs without or with a Single Lysine Residue

Lysine to arginine substitutions were applied to generate ISVDs without lysine residues. Then, one single lysine residue was introduced at variable positions, either a canonical lysine K43, K64 or K75 was reintroduced, or a lysine residue was introduced at different positions (N73K, R45K (or P45K), N73K, Q105K, K fused to the C-terminus). The ISVDs were produced in E. coli, purified using protein A chromatography, and buffer-exchanged to D-PBS. Table 14 shows the thermal stability and kinetic parameters of these single lysine residue variants.

The K_{D} of the variants was generally preserved, and the Tm differed dependent on the position of the engineered lysine. This demonstrates that substitution of lysine residues in ISVDs is generally tolerated and that lysine residues can be introduced at a preferred position. Note that in certain ISVDs, lysine residues may occur in the complementary determining regions. Obviously, to obtain an ISVD devoid of lysine residues, also these lysine residues need to be substituted with arginine residues or with any other amino acid residue that is tolerated at these positions in term of thermal stability and preservation of binding.

TABLE 14
Melting temperature and affinity constants of different single lysine
residue ISVDs variants and comparison with the precursor ISVD

| | | | | | | | |
|---|---|---|---|---|---|---|---|
| | SEQ ID | mutations vs. | Lysine | | | | |
| Variant | NO | precursor | position | Tm | ka (1/Ms) | kd (1/s) | KD (M) |

Precursor ISVD = T028501789

| | | | | | | | |
|---|---|---|---|---|---|---|---|
| T039400050 | 21 | K43R, K64R, K75R | No Lys | 76 | 5.53E+05 | 5.26E−04 | 9.52E−10 |
| T039400051 | 22 | K43R, K64R | 75 | 77 | ND | ND | ND |
| T039400054 | 25 | K43R, K64R, | 114 | 78 | 5.22E+05 | 5.26E−04 | 1.01E−09 |
| | | K75R, C-term. K | | | | | |
| T039400049 | 20 | K43R, K64R, | 105 | 78 | 5.86E+05 | 5.36E−04 | 9.14E−10 |
| | | K75R, Q105K | | | | | |
| T039400052 | 23 | K43R, K64R, | 73 | 74 | 4.41E+05 | 6.33E−04 | 1.43E−09 |
| | | N73K, K75R | | | | | |
| T039400047 | 18 | K43R, K75R | 64 | 77 | 4.93E+05 | 4.51E−04 | 9.15E−10 |
| T039400048 | 19 | K43R, R45K, | 45 | 78 | 5.77E+05 | 5.04E−04 | 8.74E−10 |
| | | K64R, K75R | | | | | |
| T039400053 | 23 | K64R, K75R | 43 | 77 | 5.47E+05 | 5.16E−04 | 9.43E−10 |
| T028501789 | 1 | None | 43, 64, 75 | 80 | 7.16E+05 | 8.85E−04 | 1.24E−09 |

Precursor ISVD = T028501805

| | | | | | | | |
|---|---|---|---|---|---|---|---|
| T039400040 | 61 | K43R, K64R, K75R | No Lys | 73 | 3.71E+05 | 2.31E−03 | 6.22E−09 |
| T039400039 | 60 | K43R, K64R | 75 | 73 | 3.01E+05 | 2.26E−03 | 7.51E−09 |
| T039400046 | 67 | K43R, K64R, | 114 | 73 | 3.77E+05 | 2.24E−03 | 5.95E−09 |
| | | K75R, C-term. K | | | | | |
| T039400044 | 65 | K43R, K64R, | 105 | 73 | 3.57E+05 | 2.29E−03 | 6.42E−09 |
| | | K75R, Q105K | | | | | |
| T039400042 | 63 | K43R, K64R, | 73 | 67 | 1.98E+05 | 1.78E−02 | 8.96E−08 |
| | | N73K, K75R | | | | | |
| T039400041 | 62 | K43R, K75R | 64 | 73 | 2.89E+05 | 2.32E−03 | 8.01E−09 |
| T039400045 | 66 | K43R, P45K, | 45 | 68 | 3.06E+05 | 2.63E−03 | 8.59E−09 |
| | | K64R, K75R | | | | | |
| T039400043 | 64 | K64R, K75R | 43 | 73 | 3.53E+05 | 2.26E−03 | 6.42E−09 |
| T028501805 | 43 | None | 43, 64, 75 | 74 | 7.72E+05 | 9.92E−04 | 1.28E−09 |

ND: Not determined

### Example 3. Binding Kinetics and Thermal Stability of an Anti-CDS ISVD without Lysine Residues

Lysine to arginine substitutions were applied to modify an anti-CD8 ISVD into a variant without lysine residues. The ISVDs were produced in E. coli, purified using protein A chromatography, and buffer-exchanged to D-PBS. Melting temperature and affinity are shown in Table 15. The impact of the mutations on thermal stability and binding kinetics was minimal.

TABLE 15
Melting temperature and affinity constants of a variant of ISVD (A-1) wherein
all lysines have been substituted and comparison with the precursor ISVD (A)

| | SEQ | | | | | | |
|---|---|---|---|---|---|---|---|
| | ID | mutations | Lysine | | ka | | |
| Variant | NO | vs. ref. | position | Tm | (1/Ms) | kd (1/s) | KD (M) |
| A- | 224 | K43R, K64R, | No Lys | 68 | 3.69E+06 | 4.18E−04 | 1.13E−10 |
| 1(A044301298) | | K75R | | | | | |
| A(A044301152) | 250 | precursor | 43, 64, 75 | 69 | 3.37E+06 | 4.12E−04 | 1.22E−10 |

### Example 4. Reactivity of Lysine Residues Differs Dependent on their Position in the ISVD Framework

Acylation of ϵ-amino group of Lysine residues with an N-hydroxy succinimide (NHS) ester is a common method for bioconjugation. To assess the relative reactivity of lysine residues depending on their position in an ISVD sequence, the TNFα antagonistic ISVD A016600058 was engineered to incorporate a total of nine lysine residues. The ISVDs were produced in E. coli, purified using protein A chromatography, and buffer-exchanged to D-PBS. The impact of the mutations was evaluated and the reactivity of the ϵ-amino group of each lysine residue and the α-amino group at the N-terminus was determined. The melting temperature of

A016600058 and the variants are depicted in Table 16. The variants showed slightly lower melting temperature (0-4° C.). R19K, R45K, R83K and Q105K were fully tolerated while N73K and R71K were less tolerated.

The functional activity of A016600058 and the variants was assessed in an TNFα reporter assay. HEK293_NFkB-NLucP cells are TNF receptor expressing cells that were stably transfected with a reporter construct encoding Nano luciferase under control of a NFkB dependent promoter. Incubation of the cells with soluble human TNFα resulted in NFkB mediated Nano luciferase gene expression. Nano luciferase luminescence was measured using Nano-Glo Luciferase substrate mixed with lysing buffer at the ratio of 1:50 added onto cells. Samples were mixed 5 min on a shaker to obtain complete lysis. Glo response™ HEK293_NFkB-NLucP cells were seeded at 20000 cells/well in growth medium in white tissue culture (TC) treated 96-well plates with transparent bottom. Dilution series of A016600058 and variants were added to 25 pM human TNFα and incubated with the cells for 5 hours at 37° C. A016600058 inhibited human TNFα-induced NFkB activation in a concentration-dependent manner. The IC50 of A016600058 and the variants is depicted in Table 16. The mutations did not alter the IC50 more than 2-fold, except when R71K and N73K were introduced in the same variant.

TABLE 16
Melting temperature and functional activity of A016600058 (precursor) and variants
of A016600058 with up to nine lysine residues (“ID” indicates the SEQ ID NO)

| | | | | Average | | |
|---|---|---|---|---|---|---|
| | | lysine positions | | Tm (° C.) | Average of | Average of |
| Variant | mutations vs. ref. | (Kabat) | ID | at pH 7 | ka (1/Ms) | kd (1/s) |
| T031200256 | N73K, R83K | 43, 64, 73, 75, 83 | 69 | 63 | 4.3E−11 | 1.11 |
| T031200257 | R19K, N73K, R83K | 19, 43, 64, 73, 75, 83 | 70 | 63 | 4.93E−11 | 1.28 |
| T031200258 | R19K, R83K, Q105K | 19, 43, 64, 75, 83, 105 | 71 | 66 | 6.09E−11 | 1.58 |
| T031200260 | R19K, N73K, R83K, | 19, 43, 64, 73, 75, 83, 105 | 73 | 63 | 5.34E−11 | 1.38 |
| | Q105K | | | | | |
| T031200259 | R19K, R71K, R83K, | 19, 43, 64, 71, 75, 83, 105 | 72 | 64 | 4.82E−11 | 1.25 |
| | Q105K | | | | | |
| T031200262 | R19K, R45K, N73K, | 19, 43, 45, 64, 73, 75, 83, 105 | 75 | 63 | 6.43E−11 | 1.67 |
| | R83K, Q105K | | | | | |
| T031200261 | R19K, R71K, N73K, | 19, 43, 64, 71, 73, 75, 83, 105 | 74 | 62 | 1.91E−10 | 6.41 |
| | R83K, Q105K | | | | | |
| T031200263 | R19K, R45K, R71K, | 19, 43, 45, 64, 71, 73, 75, 83, 105 | 78 | 62 | 1.92E−10 | 6.47 |
| | N73K, R83K, Q105K | | | | | |
| A016600058 | precursor | 43, 64, 75 | 68 | 66 | 3.86E−11- | — |
| | | | | | 2.97E−11 | |

The ISVD with 9 lysine groups (ID T031200263) was subjected to a reaction with EZ-Link Sulfo-5 NHS-LC Biotin (Thermo Scientific, catn A39257). The ISVD (150 μl at 233.7 μM in PBS) was reacted with either 7.0, 24.5 and 70.0 μl EZ-Link Sulfo-NHS-LC Biotin (10 mM dissolved in H₂O), or with respectively 2, 7 and 20-fold excess of the biotin reagent. The reaction was incubated on ice for 2 h and dialyzed to PBS on a Slide-A-lyzer MINI Dialysis 0.5 ml device 3.5K (Thermo Fisher, catn 88400).

The conjugation level of the different amine groups was evaluated by peptide mapping using AspN digestion, peptides separation by reversed phase chromatography and MS/MS detection. The results of the peptide mapping analysis are presented in Table 16. Due to the proximity of K43-K45 and K73-K75, it was not always possible to discriminate which K was conjugated using MS/MS data, resulting in the given ranges.

TABLE 17
Conjugation level of the different amine groups on A016600058with
9 lysines as evaluated by peptide mapping

| | Conjugation level (%) | | | |
|---|---|---|---|---|
| Position amino group | Control | 2x ME | 7x ME | 20x ME |
| N-terminus | 0.0 | 3.1 | 13.0 | 70.7 |
| K19 | 0.0 | 4.8 | 17.9 | 72.4 |
| K43 | 0.0 | 0.1 | 1.7 | 15.3-25.4 |
| K45 | 0.0 | 23.6 | 70.9 | 89.9-100 |
| K64 | 0.0 | 3.8 | 12.4 | 53.3 |
| K71 | 0.0 | 0.9 | 3.3 | 12.6 |
| K73 | 0.0 | 5.7-7.3 | 20.6-25.8 | 70.4-82.9 |
| K75 | 0.0 | 0.1-1.7 | 1.0-6.2 | 24.2-36.8 |
| K83 | 0.0 | 1.4 | 5.5 | 28.3 |
| K105 | 0.0 | 2.6 | 28.0 | 77.1 |

### Example 5. Introduction of a Unique Amine Group into Amine-Free ISVDs

Description of the Amine-Free ISVDs and Variants with a Unique Amine Group

Amine-free ISVDs were created by modifying three ISVDs (referred to as precursor ISVDs in Table 18), substituting each lysine residue with arginine (indicated by “all_K→R”), and substituting the N-terminal residue with glutamine, allowing pyroglutamation of the N-terminus (indicated by E1Q). Taking the three amine-free ISVDs as a basis, twenty-one variants were created with a re-introduced amine group, by either introducing a single lysine residue (through substitutions at variant positions) or by fusing a glycine to the N-terminus to regenerate a free N-terminal amine. The ISVDs were produced in E. coli, purified using protein A chromatography, and buffer-exchanged to D-PBS (Biointron).

Thermal Stability of Amine-Free ISVDs and Variants with a Unique Amine Group

The thermal stability was assessed as described in example 1. The ATm was calculated relative to the precursor ISVD. The Tm of the three amine-free ISVDs differed less than 1° C. from that of the precursor ISVDs. From the variants with a unique amino group, the largest impact was generally observed for the substitution of the residue at kabat position 12 (Table 18).

Bio-Conjugation of Amine-Free ISVDs and Variants with a Unique Amine Group

A 3× molar excess of a decanoic acid-NHS ester (Broadpharm, catn BP-24338) was reacted with a 1.5 mg/ml ISDV solution. This relatively low excess of probe was selected to achieve a non-saturating concentration, enabling the ranking of bio-conjugation susceptibility for each variant. The reaction mixtures were incubated at room temperature for 30 minutes.

### LC-MS Analysis for Assessing the Extent of Bio-Conjugation

Liquid chromatography-mass spectrometry (LC-MS) analyses were performed using a Waters MassPrep™ Micro desalting column (Waters) and a Q Exactive Plus Orbitrap mass spectrometer (Thermo Scientific). A gradient from 5 to 90% B in 1.5 min was applied, with 0.1% formic acid in water as mobile phase A, and 0.1% formic acid in acetonitrile as mobile phase B. The degree of bio-conjugation was determined on the pyroglutamated variant using the MS signal intensity.

The bio-conjugation efficiency of the variants was clearly dependent on the position of the lysine in the ISVD framework. The lysine at position 45 (kabat) was most susceptible, followed by position 108 and 73 (Table 18).

TABLE 18
Conjugation level of the ISVDs with a unique amino group

| | | | | | | |
|---|---|---|---|---|---|---|
| | SEQ | | amino | | | |
| | ID | | group | | | % |
| ID | NO | Description | position | Tm | ΔTm | conjugated |

Precursor ISVD = TPP-134228

| | | | | | | |
|---|---|---|---|---|---|---|
| TPP-134228 | 226 | Precursor | multiple | 64 | 0.0 | ND |
| TPP-134229 | 174 | G-Precursor(all_K→R, E1Q) | N-terminus | 62 | 2.4 | 44.7 |
| TPP-134230 | 175 | Precursor(all_K→ m, R, E1Q) | none | 64 | 0.5 | / |
| TPP-134241 | 186 | Precursor(all_K→R, E1Q, E5K) | 5 | 66 | −1.8 | 54.7 |
| TPP-134244 | 189 | Precursor(all_K→R, E1Q, S7K) | 7 | 63 | 1.1 | 50.6 |
| TPP-134233 | 178 | Precursor(all_K→R, E1Q, G10K) | 10 | 61 | 3.3 | 58.8 |
| TPP-134236 | 181 | Precursor(all_K→R, E1Q, V12K) | 12 | 55 | 8.9 | 65.2 |
| TPP-134237 | 182 | Precursor(all_K→R, E1Q, Q13K) | 13 | 64 | −0.1 | 46.2 |
| TPP-134232 | 177 | Precursor(all_K→R, E1Q, R19K) | 19 | 62 | 2.5 | 45.8 |
| TPP-134238 | 183 | Precursor(all_K→R, E1Q, R43K) | 43 | 63 | 0.9 | 44.1 |
| TPP-134239 | 184 | Precursor(all_K→R, E1Q, E44K) | 44 | 63 | 1.4 | 72.4 |
| TPP-134240 | 185 | Precursor(all_K→R, E1Q, R45K) | 45 | 65 | −0.8 | 90.1 |
| TPP-134242 | 187 | Precursor(all_K→R, E1Q, D61K) | 61 | 62 | 2.3 | 66.6 |
| TPP-134243 | 188 | Precursor(all_K→R, E1Q, R64K) | 64 | 63 | 0.9 | 70.0 |
| TPP-134245 | 190 | Precursor(all_K→R, E1Q, S70K) | 70 | 60 | 4.4 | 47.5 |
| TPP-134246 | 191 | Precursor(all_K→R, E1Q, R71K) | 71 | 62 | 2.2 | 36.5 |
| TPP-134247 | 192 | Precursor(all_K→R, E1Q, N73K) | 73 | 63 | 1.4 | 68.8 |
| TPP-134248 | 193 | Precursor(all_K→R, E1Q, R75K) | 75 | 64 | 0.5 | 41.8 |
| TPP-134249 | 194 | Precursor(all_K→R, E1Q, R83K) | 83 | 64 | 0.3 | 59.9 |
| TPP-134234 | 179 | Precursor(all_K→R, E1Q, Q105K) | 105 | 65 | −0.6 | 47.0 |
| TPP-134235 | 180 | Precursor(all_K→R, E1Q, Q108K) | 108 | 63 | 1.1 | 80.0 |
| TPP-134231 | 176 | Precursor(all_K→R, E1Q)-K | C-terminal | 63 | 1.0 | 63.1 |
| | | | residue | | | |

Precursor = T042500044

| | | | | | | |
|---|---|---|---|---|---|---|
| TPP-134251 | 197 | Precursor(E1Q) | multiple | 77 | 0.0 | ND |
| TPP-134252 | 198 | G-Precursor(all_K→R, E1Q) | N-terminus | 76 | 1.4 | 39.0 |
| TPP-134253 | 199 | Precursor(all_K→R, E1Q) | none | 76 | 0.8 | / |
| TPP-134254 | 200 | Precursor(all_K→R, E1Q, E5K) | 5 | 75 | 1.7 | 59.0 |
| TPP-134255 | 201 | Precursor(all_K→R, E1Q, S7K) | 7 | 75 | 1.9 | 57.8 |
| TPP-134256 | 202 | Precursor(all_K→R, E1Q, G10K) | 10 | 74 | 2.9 | 50.8 |
| TPP-134257 | 203 | Precursor(all_K→R, E1Q, V12K) | 12 | 69 | 8.4 | 56.1 |
| TPP-134258 | 204 | Precursor(all_K→R, E1Q, Q13K) | 13 | 77 | 0.0 | 52.6 |
| TPP-134259 | 205 | Precursor(all_K→R, E1Q, R19K) | 19 | 75 | 1.5 | 50.6 |
| TPP-134260 | 206 | Precursor(all_K→R, E1Q, R43K) | 43 | 77 | 0.3 | 47.1 |
| TPP-134261 | 207 | Precursor(all_K→R, E1Q, E44K) | 44 | 76 | 0.7 | 58.4 |
| TPP-134262 | 208 | Precursor(all_K→R, E1Q, R45K) | 45 | 76 | 1.3 | 76.8 |
| TPP-134263 | 209 | Precursor(all_K→R, E1Q, D61K) | 61 | 75 | 1.7 | 61.0 |
| TPP-134264 | 210 | Precursor(all_K→R, E1Q, R64K) | 64 | 76 | 0.9 | 44.9 |
| TPP-134265 | 211 | Precursor(all_K→R, E1Q, S70K) | 70 | 75 | 2.0 | 53.3 |
| TPP-134266 | 212 | Precursor(all_K→R, E1Q, R71K) | 71 | 74 | 2.6 | 41.0 |
| TPP-134267 | 213 | Precursor(all_K→R, E1Q, N73K) | 73 | 75 | 1.6 | 61.4 |
| TPP-134268 | 214 | Precursor(all_K→R, E1Q, R75K) | 75 | 77 | 0.2 | 47.0 |
| TPP-134269 | 215 | Precursor(all_K→R, E1Q, R83K) | 83 | 76 | 1.3 | 53.6 |
| TPP-134270 | 216 | Precursor(all_K→R, E1Q, Q105K) | 105 | 77 | −0.2 | 58.7 |
| TPP-134271 | 217 | Precursor(all_K→R, E1Q, Q108K) | 108 | 76 | 1.3 | 73.7 |
| TPP-134250 | 196 | Precursor(all_K→R, E1Q)-K | C-terminal | 77 | 0.3 | 50.1 |
| | | | residue | | | |

Precursor = ALB00622/T028501805

| | | | | | | |
|---|---|---|---|---|---|---|
| TPP-134274 | 228 | Precursor(E1Q) | multiple | 75 | 0.0 | ND |
| TPP-134273 | 249 | G-Precursor(all_K→R, E1Q) | N-terminus | 74 | 1.1 | 45.9 |
| TPP-134275 | 229 | Precursor(all_K→R, E1Q) | none | 74 | 0.5 | — |
| TPP-134280 | 234 | Precursor(all_K→R, E1Q, E5K) | 5 | 73 | 1.5 | 60.5 |
| TPP-134281 | 235 | Precursor(all_K→R, E1Q, S7K) | 7 | 72 | 2.2 | 76.4 |
| TPP-134276 | 230 | Precursor(all_K→R, E1Q, G10K) | 10 | 73 | 2.1 | 62.5 |
| TPP-134282 | 236 | Precursor(all_K→R, E1Q, V12K) | 12 | 68 | 6.2 | 68.6 |
| TPP-134277 | 231 | Precursor(all_K→R, E1Q, Q13K) | 13 | 75 | −0.3 | 49.0 |
| TPP-134283 | 237 | Precursor(all_K→R, E1Q, R19K) | 19 | 74 | 0.8 | 48.4 |
| TPP-134278 | 232 | Precursor(all_K→R, E1Q, R43K) | 43 | 74 | 0.5 | 48.5 |
| TPP-134284 | 238 | Precursor(all_K→R, E1Q, E44K) | 44 | 74 | 0.1 | 71.1 |
| TPP-134285 | 239 | Precursor(all_K→R, E1Q, R45K) | 45 | 69 | 5.1 | 79.2 |
| TPP-134286 | 240 | Precursor(all_K→R, E1Q, D61K) | 61 | 73 | 2.1 | 74.8 |
| TPP-134287 | 241 | Precursor(all_K→R, E1Q, R64K) | 64 | 74 | 0.4 | 63.5 |
| TPP-134288 | 242 | Precursor(all_K→R, E1Q, S70K) | 70 | 71 | 3.9 | 49.4 |
| TPP-134289 | 243 | Precursor(all_K→R, E1Q, R71K) | 71 | 70 | 4.9 | 11.3 |
| TPP-134290 | 244 | Precursor(all_K→R, E1Q, N73K) | 73 | 68 | 6.9 | 69.6 |
| TPP-134279 | 233 | Precursor(all_K→R, E1Q, R75K) | 75 | 74 | 0.2 | 55.0 |
| TPP-134291 | 245 | Precursor(all_K→R, E1Q, R83K) | 83 | 74 | 0.5 | 64.9 |
| TPP-134292 | 246 | Precursor(all_K→R, E1Q, Q105K) | 105 | 75 | −0.3 | 69.3 |
| TPP-134293 | 247 | Precursor(all_K→R, E1Q, Q108K) | 108 | 73 | 1.7 | 89.0 |
| TPP-134272 | 248 | Precursor(all_K→R, E1Q)-K | C-terminal | 74 | 0.4 | 65.3 |
| | | | residue | | | |

### Example 6. Bio-Conjugation of ISVDs with Two Amine Groups Strategically Introduced at Optimal Positions

Description of an Amine-Free ISVD and Variants with Reintroduced Amine Groups

An amine-free ISVD was created by modifying ISVD T042500096, substituting each lysine residue with arginine, and substituting the N-terminal residue with glutamine to allow pyroglutamation. Variants with two amine groups were created by introducing lysines at position 45 and 108 or 73 or 13. A variant with only one amine group at position 45 was included as a control.

Another ISVD with two amine groups was created from precursor T042500044 (see example 5) by introducing two lysines in the amine-free variant TPP-134253 at positions 45 and 73.

Bio-Conjugation of ISVDs with Two Amine Groups

A 5× molar excess of EZ-Link Sulfo-NHS-LC Biotin (Thermo Scientific, catn A39257) was reacted with a 1.5 mg/ml ISDV solution. The reaction mixtures were incubated at room temperature for 30 minutes.

### LC/MS Analysis for Assessing the Extent of Bio-Conjugation

The same protocol as in Example 4 was followed, with only the pyroglutamated fraction considered for calculating the percentage of bio-conjugate. The bio-conjugation efficiency of the variants aligned with our previous data from ISVDs containing a single amine group. The control molecule, which has a single amine group at position 45, was efficiently conjugated once, while combinations of amine groups at positions 108 and 73 resulted in efficient double conjugations. As expected, the combination of an amine group at position 45 with one at position 13, expected to be a less favourable position (see Example 5), indeed produced significantly fewer double conjugations (Table 19).

TABLE 19
Biotinylation level of the ISVDs with one or two amine groups

| | | | | N° of biotin | | |
|---|---|---|---|---|---|---|
| | | | Lysine | conjugations (%) | | |
| ID | SEQ ID NO | Description | position | 0 | 1 | 2 |

Precursor ISVD = T042500096

| | | | | | | |
|---|---|---|---|---|---|---|
| T03230100013 | 220 | Precursor(all_K→R, E1Q, | 45.108 | 0.4 | 18.8 | 77.6 |
| | | R45K, Q108K) | | | | |
| T03230100014 | 221 | Precursor(all_K→R, E1Q, | 45.73 | 1.1 | 27.7 | 67.7 |
| | | R45K, N73K) | | | | |
| T03230100033 | 222 | Precursor(all_K→R, E1Q, | 13.45 | 3.6 | 59.0 | 35.8 |
| | | Q13K, R45K) | | | | |
| T03230100032 | 223 | Precursor(all_K→R, E1Q, | 45 | 3.4 | 92.6 | / |
| | | R45K) | | | | |

TABLE A-1
Preferred Linker sequences of the
technology

| | SEQ | |
|---|---|---|
| Name of | ID | Amino acid |
| linker | NO: | sequences |
| A3 (3A) | 107 | AAA |
| 5GS linker | 108 | GGGGS |
| 7GS linker | 109 | SGGSGGS |
| 8GS linker | 110 | GGGGSGGS |
| 9GS linker | 111 | GGGGSGGGS |
| 10GS linker | 112 | GGGGSGGGGS |
| 15GS linker | 113 | GGGGSGGGGSGGGGS |
| 18GS linker | 114 | GGGGSGGGGSGGGGSGGS |
| 20GS linker | 115 | GGGGSGGGGSGGGGSGGGGS |
| 25GS linker | 116 | GGGGSGGGGSGGGGSGGGGSGGGGS |
| 30GS linker | 117 | GGGGSGGGGSGGGGSGGGGSGGGGS |
| | | GGGGS |
| 35GS linker | 118 | GGGGSGGGGSGGGGSGGGGSGGGGS |
| | | GGGGSGGGGS |
| 40GS linker | 119 | GGGGSGGGGSGGGGSGGGGGGGGSG |
| | | GGGSGGGGSGGGGS |
| G1 hinge | 120 | EPKSCDKTHTCPPCP |
| 9GS-G1 hinge | 121 | GGGGSGGGSEPKSCDKTHTCPPCP |
| Llama upper | 122 | EPKTPKPQPAAA |
| long hinge | | |
| region | | |
| G3 hinge | 123 | ELKTPLGDTTHTCPRCPEPKSCDTP |
| | | PPCPRCPEPKSCDTPPPCPRCPEPK |
| | | SCDTPPPCPRCP |

### Items of the Present Technology

The present technology provides the following items:
1. A molecule comprising or consisting of at least one immunoglobulin single variable domain (ISVD), wherein the at least one ISVD:
   comprises at least one (engineered) lysine at at least one solvent accessible position, wherein the solvent accessible position is not located at positions 43, 64, 75 and/or 83 (according to Kabat), preferably wherein the solvent accessible position is not located at positions 43, 64, 75, 83 and/or 97 (according to Kabat), even more preferably wherein the solvent accessible position is not located at positions 19, 43, 64, 75, 83, 97 and/or 105 (according to Kabat); or
   comprises only two lysines in its amino acid sequence, wherein the two lysines are located at positions 43, 64, 75 and/or 83 (according to Kabat);
   comprises a single lysine, wherein the single lysine is located at position 43, 64, 75 or 83 (according to Kabat); or
   does not comprise any lysine in its sequence, preferably does not comprise any primary amine in its sequence.
2. The molecule according to item 1, wherein the ISVD has a globular three-dimensional (3D) structure and wherein the ISVD has a solubility of 10 mg/ml or more, preferably of 20 mg/ml, preferably of 50 mg/ml or more, and even more preferably of 100 mg/mL, measured in an aqueous solution at room temperature, preferably wherein the aqueous solution is citrate buffer 5 mM or PBS, at pH 7.0 or 7.4.
3. The molecule according to item 1 or 2, wherein the at least one ISVD does not comprise any lysine in its sequence and wherein one or more, or preferably all, of positions 43, 64 and 75 have an amino acid selected from glutamine, histidine, arginine, alanine, and glutamate, preferably arginine, preferably wherein the ISVD does not comprise any primary amine in its sequence.
4. The molecule according to any one of items 1-3, wherein the at least one ISVD does not comprise any lysine in its sequence and wherein one or more, or preferably all, of positions 43, 64, 75 and 83 have an amino acid selected from glutamine, histidine, arginine, alanine, and glutamate, preferably arginine.
5. The molecule according to any one of items 1-4, wherein the at least one ISVD does not comprise any lysine in its sequence and wherein one or more, or preferably all, of positions 43, 64, 75 and 83 have an amino acid selected from glutamine, histidine, arginine and glutamate, preferably arginine.
6. The molecule according to any one of items 1-2 wherein the ISVD is suitable for primary amine site conjugation.
7. The molecule according to any one of items 1-2 and 5-6, wherein the at least one ISVD comprises at least one (engineered) lysine at at least one solvent accessible position, preferably wherein the solvent accessible position is not located in at least one of the CDRs of the ISVD.
8. The molecule according to any one of items 1-2 and 5-7, wherein the at least one ISVD comprises at least one lysine and is suitable for site-specific conjugation on at least one primary amine present on the side chain of the at least one lysine and/or at the N-terminal primary amine.
9. The molecule according to any one of items 1-8, wherein the at least one ISVD comprises at least one or at least two amino acids distinct from lysine, respectively at at least one or at at least two of positions 43, 64 and 75 (according to Kabat), preferably wherein the at least one or at least two amino acids distinct from lysine are selected from glutamine, histidine, arginine, alanine, or glutamate, more preferably from glutamine, histidine, arginine, or glutamate, even more preferably at least one arginine.
10. The molecule according to any one of items 1-2 and 5-9, wherein the at least one ISVD comprises at least one lysine and all lysines are engineered lysines, preferably located in the ISVD at a position different from position 43, 64, and/or 75, more preferably located in the ISVD at a position different from position 43,64, 75 and/or 83, even more preferably located in the ISVD at a position different from position 19, 43, 64, 75, 83, 97 and/or 105 (according to Kabat).
11. The molecule according to any one of items 1-2 and 5-10, wherein the at least one ISVD comprises a single lysine, preferably a single engineered lysine.
12. The molecule according to any one of items 1-2 and 5-11, wherein the at least one ISVD comprises more than one lysine, such as at least 2, at least 3, at least 4, at least 5, at least 6, at least 7, at least 8 or at least 9 lysines, preferably wherein at least one, or at least 2, or at least 3, or at least 4, or at least 5, or at least 6, or at least 7, or at least 8, or at least 9 lysines are engineered lysines.
13. The molecule according to any one of items 1-2 and 5-12, wherein the at least one ISVD comprises at least one lysine at at least one of the following positions: 5, 7, 10, 12, 13, 19, 43, 44, 45, 54, 61, 64, 70, 71, 73, 75, 83, 85, 95, 105 and/or 108 (according to Kabat).
14. The molecule according to item 13, wherein the at least one ISDV comprises at least one lysine at at least one of the following positions: 19, 43, 45, 64, 71, 73, 75, 83, 105 and/or 108 (according to Kabat), preferably at positions 45, 73 and 108, such as at positions 43 and/or 45 (according to Kabat).
15. The molecule according to any one of items 1-2 and 5-14 wherein the at least one ISDV comprises at least one lysine at at least one of the following positions: 45, 71, 73, 105 and/or 108 (according to Kabat), preferably at positions 45, 73 and 108, and wherein the ISVD does not comprise any lysine at positions 19, 43, 64, 75, 83, 97 and 105.
16. The molecule according to any one of items 1-2 and 5-15, wherein the at least one ISDV comprises a C-terminal lysine and/or a N-terminal lysine.
17. The molecule according to any one of items 1-16, wherein the at least one ISDV comprises a glutamine at position 1 (according to Kabat), preferably wherein the N-terminal of the at least one ISVD comprises a pyroglutamate modification.
18. The molecule according to item 17, wherein the at least one ISDV does not comprise any primary amine.
19. The molecule according to any one of items 1-18, wherein the at least one ISDV does not comprise any lysine in its sequence and wherein the N-terminal of the at least one ISVD comprises a pyroglutamate modification.
20. The molecule according to any one of items 1-19, wherein the at least one ISVD does not specifically bind to any human protein or binds one or more human proteins with a K_{D} greater than 5×10⁻⁴ mol/litre, preferably wherein the at least one ISVD does also not specifically bind to any non-protein molecule, such as nucleic acids (e.g., DNA, RNA), lipids or glycans, or binds one or more non-protein molecules with a K_{D} greater than 5×104 mol/litre.
21. The molecule according to any one of items 1-20, wherein the at least one ISVD is derived from a V_{H}, a humanized V_{H}, a human V_{H}, a V_{HH}, a humanized V_{HH}, a domain antibody (dAb), or a camelized V_{H}, preferably derived from an ISVD belonging to the “V_{H}3 class”.
22. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 79 (or has 3, 2 or 1 amino acid difference(s) with SEQ ID NO: 79);
   CDR2 comprises the amino acid sequence of SEQ ID NO: 80 (or has 3, 2 or 1 amino acid difference(s) with SEQ ID NO: 80); and
   CDR3 comprises the amino acid sequence of SEQ ID NO: 81 (or has 3, 2 or 1 amino acid difference(s) with SEQ ID NO: 81);
wherein the CDR sequences are determined according to AbM definition.
23. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 264 (TYTMG) (or has 3, 2 or 1
   CDR2 comprises the amino acid sequence of SEQ ID NO: 265 (AIIWSGSNTYYADSVHG) (or has 3, 2 or 1 amino acid difference(s) with SEQ ID NO: 265); and
   CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.
24. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 264 (TYTMG) (or has 3, 2 or 1 amino acid difference(s) with SEQ ID NO: 264);
   CDR2 comprises the amino acid sequence of SEQ ID NO: 304 (AIIWSGSNTYYADSVKG) (or has 3, 2 or 1 amino acid difference(s) with SEQ ID NO: 304); and
   CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.
25. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 264 (TYTMG) (or has 3, 2 or 1 amino acid difference(s) with SEQ ID NO: 264);
   CDR2 comprises the amino acid sequence of SEQ ID NO: 281 (AIIWSGSNTYYADSVRG) (or has 3, 2 or 1 amino acid difference(s) with SEQ ID NO: 281); and
   CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.
26. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 264 (TYTMG) (or has 3, 2 or 1 amino acid difference(s) with SEQ ID NO: 264);
   CDR2 comprises the amino acid sequence of SEQ ID NO: 280 (AIIWSGSNTYYADSVEG) (or has 3, 2 or 1 amino acid difference(s) with SEQ ID NO: 280); and
   CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.
27. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 264 (TYTMG) (or has 3, 2 or 1 amino acid difference(s) with SEQ ID NO: 264);
   CDR2 comprises the amino acid sequence of SEQ ID NO: 282 (AIIWSGSNTYYADSVQG) (or has 3, 2 or 1 amino acid difference(s) with SEQ ID NO: 282); and
   CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.
28. The molecule according to any one of items 1-21, wherein the at least one ISVD comprises or consists of three complementarity determining regions (CDR1 to CDR3, respectively), wherein:
   CDR1 comprises the amino acid sequence of SEQ ID NO: 264 (TYTMG) (or has 3, 2 or 1 amino acid difference(s) with SEQ ID NO: 264);
   CDR2 comprises the amino acid sequence of SEQ ID NO: 283 (AIIWSGSNTYYADSVAG) (or has 3, 2 or 1 amino acid difference(s) with SEQ ID NO: 283); and
   CDR3 comprises the amino acid sequence of SEQ ID NO: 81 (QHFGPIGLTTRGYHY) (or has 3, 2 or 1 amino acid difference(s) with SEQ ID NO: 81);
wherein the CDR sequences are determined according to Kabat numbering.
29. The molecule according to any one of items 1-21, wherein the at least one ISVD comprises three complementarity determining regions (CDR1 to CDR3, respectively), wherein:
   CDR1 comprises the amino acid sequence of SEQ ID NO: 82 (or has 3, 2 or 1 amino acid difference(s) with SEQ ID NO: 82);
   CDR2 comprises the amino acid sequence of SEQ ID NO: 83 (or has 3, 2 or 1 amino acid difference(s) with SEQ ID NO: 83); and
   CDR3 comprises the amino acid sequence of SEQ ID NO: 84 (or has 3, 2 or 1 amino acid difference(s) with SEQ ID NO: 84);
wherein the CDR sequences are determined according to AbM definition.
30. The molecule according to any one of items 1-21, wherein the at least one ISVD comprises three complementarity determining regions (CDR1 to CDR3, respectively), wherein:
   CDR1 comprises the amino acid sequence of SEQ ID NO: 266 (EYALG) (or has 3, 2 or 1 amino acid difference(s) with SEQ ID NO: 266);
   CDR2 comprises the amino acid sequence of SEQ ID NO: 267 (AlNWGGGWTYYADSVKG) (or has 3, 2 or 1 amino acid difference(s) with SEQ ID NO: 267); and
   CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.
31. The molecule according to any one of items 1-21, wherein the at least one ISVD comprises three complementarity determining regions (CDR1 to CDR3, respectively), wherein:
   CDR1 comprises the amino acid sequence of SEQ ID NO: 266 (EYALG) (or has 3, 2 or 1 amino acid difference(s) with SEQ ID NO: 266);
   CDR2 comprises the amino acid sequence of SEQ ID NO: 284 (AlNWGGGWTYYADSVEG) (or has 3, 2 or 1 amino acid difference(s) with SEQ ID NO: 284); and
   CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.
32. The molecule according to any one of items 1-21, wherein the at least one ISVD comprises three complementarity determining regions (CDR1 to CDR3, respectively), wherein:
   CDR1 comprises the amino acid sequence of SEQ ID NO: 266 (EYALG) (or has 3, 2 or 1 amino acid difference(s) with SEQ ID NO: 266);
   CDR2 comprises the amino acid sequence of SEQ ID NO: 285 (AlNWGGGWTYYADSVQG) (or has 3, 2 or 1 amino acid difference(s) with SEQ ID NO: 285); and
   CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.
33. The molecule according to any one of items 1-21, wherein the at least one ISVD comprises three complementarity determining regions (CDR1 to CDR3, respectively), wherein:
   CDR1 comprises the amino acid sequence of SEQ ID NO: 266 (EYALG) (or has 3, 2 or 1 amino acid difference(s) with SEQ ID NO: 266);
   CDR2 comprises the amino acid sequence of SEQ ID NO: 286 (AlNWGGGWTYYADSVRG) (or has 3, 2 or 1 amino acid difference(s) with SEQ ID NO: 286); and
   CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.
34. The molecule according to any one of items 1-21, wherein the at least one ISVD comprises three complementarity determining regions (CDR1 to CDR3, respectively), wherein:
   CDR1 comprises the amino acid sequence of SEQ ID NO: 266 (EYALG) (or has 3, 2 or 1 amino acid difference(s) with SEQ ID NO: 266);
   CDR2 comprises the amino acid sequence of SEQ ID NO: 287 (AlNWGGGWTYYADSVHG) (or has 3, 2 or 1 amino acid difference(s) with SEQ ID NO: 287); and
   CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.
35. The molecule according to any one of items 1-21, wherein the at least one ISVD comprises three complementarity determining regions (CDR1 to CDR3, respectively), wherein:
   CDR1 comprises the amino acid sequence of SEQ ID NO: 266 (EYALG) (or has 3, 2 or 1 amino acid difference(s) with SEQ ID NO: 266);
   CDR2 comprises the amino acid sequence of SEQ ID NO: 288 (AlNWGGGWTYYADSVAG) (or has 3, 2 or 1 amino acid difference(s) with SEQ ID NO: 288); and
   CDR3 comprises the amino acid sequence of SEQ ID NO: 84 (SSDYAGGNPTGYPY) (or has 3, 2 or 1 amino acid difference(s) with SEQ ID NO: 84);
wherein the CDR sequences are determined according to Kabat numbering.
36. The molecule according to any one of items 1-21, wherein the at least one ISVD comprises three complementarity determining regions (CDR1 to CDR3, respectively), wherein:
   CDR1 comprises the amino acid sequence of SEQ ID NO: 85 (or has 3, 2 or 1 amino acid difference(s) with SEQ ID NO: 85);
   CDR2 comprises the amino acid sequence of SEQ ID NO: 86 (or has 3, 2 or 1 amino acid difference(s) with SEQ ID NO: 86); and
   CDR3 comprises the amino acid sequence of SEQ ID NO: 87 (or has 3, 2 or 1 amino acid difference(s) with SEQ ID NO: 87);
wherein the CDR sequences are determined according to AbM definition.
37. The molecule according to any one of items 1-21, wherein the at least one ISVD comprises three complementarity determining regions (CDR1 to CDR3, respectively), wherein:
   CDR1 comprises the amino acid sequence of SEQ ID NO: 268 (SFGMS) (or has 3, 2 or 1 amino acid difference(s) with SEQ ID NO: 268);
   CDR2 comprises the amino acid sequence of SEQ ID NO: 269 (SISGSGSDTLYADSVKG) (or has 3, 2 or 1 amino acid difference(s) with SEQ ID NO: 269); and
   CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.
38. The molecule according to any one of items 1-21, wherein the at least one ISVD comprises three complementarity determining regions (CDR1 to CDR3, respectively), wherein:
   CDR1 comprises the amino acid sequence of SEQ ID NO: 268 (SFGMS) (or has 3, 2 or 1 amino acid difference(s) with SEQ ID NO: 268);
   CDR2 comprises the amino acid sequence of SEQ ID NO: 289 (SISGSGSDTLYADSVEG) (or has 3, 2 or 1 amino acid difference(s) with SEQ ID NO: 289); and
   CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.
39. The molecule according to any one of items 1-21, wherein the at least one ISVD comprises three complementarity determining regions (CDR1 to CDR3, respectively), wherein:
   CDR1 comprises the amino acid sequence of SEQ ID NO: 268 (SFGMS) (or has 3, 2 or 1 amino acid difference(s) with SEQ ID NO: 268);
   CDR2 comprises the amino acid sequence of SEQ ID NO: 290 (SISGSGSDTLYADSVQG) (or has 3, 2 or 1 amino acid difference(s) with SEQ ID NO: 290); and
   CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.
40. The molecule according to any one of items 1-21, wherein the at least one ISVD comprises three complementarity determining regions (CDR1 to CDR3, respectively), wherein:
   CDR1 comprises the amino acid sequence of SEQ ID NO: 268 (SFGMS) (or has 3, 2 or 1 amino acid difference(s) with SEQ ID NO: 268);
   CDR2 comprises the amino acid sequence of SEQ ID NO: 291 (SISGSGSDTLYADSVHG) (or has 3, 2 or 1 amino acid difference(s) with SEQ ID NO: 291); and
   CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.
41. The molecule according to any one of items 1-21, wherein the at least one ISVD comprises three complementarity determining regions (CDR1 to CDR3, respectively), wherein:
   CDR1 comprises the amino acid sequence of SEQ ID NO: 268 (SFGMS) (or has 3, 2 or 1 amino acid difference(s) with SEQ ID NO: 268);
   CDR2 comprises the amino acid sequence of SEQ ID NO: 292 (SISGSGSDTLYADSVRG) (or has 3, 2 or 1 amino acid difference(s) with SEQ ID NO: 292); and
   CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.
42. The molecule according to any one of items 1-21, wherein the at least one ISVD comprises three complementarity determining regions (CDR1 to CDR3, respectively), wherein:
   CDR1 comprises the amino acid sequence of SEQ ID NO: 268 (SFGMS) (or has 3, 2 or 1 amino acid difference(s) with SEQ ID NO: 268);
   CDR2 comprises the amino acid sequence of SEQ ID NO: 293 (SISGSGSDTLYADSVAG) (or has 3, 2 or 1 amino acid difference(s) with SEQ ID NO: 293); and
   CDR3 comprises the amino acid sequence of SEQ ID NO: 87 (GGSLSR) (or has 3, 2 or 1 amino acid difference(s) with SEQ ID NO: 87);
wherein the CDR sequences are determined according to Kabat numbering.
43. The molecule according to any one of items 1-21, wherein the at least one ISVD comprises three complementarity determining regions (CDR1 to CDR3, respectively), wherein:
   CDR1 comprises the amino acid sequence of SEQ ID NO: 88 (or has 3, 2 or 1 amino acid difference(s) with SEQ ID NO: 88);
   CDR2 comprises the amino acid sequence of SEQ ID NO: 89 (or has 3, 2 or 1 amino acid difference(s) with SEQ ID NO: 89); and
   CDR3 comprises the amino acid sequence of SEQ ID NO: 90 (or has 3, 2 or 1 amino acid difference(s) with SEQ ID NO: 90);
wherein the CDR sequences are determined according to AbM definition.
44. The molecule according to any one of items 1-21, wherein the at least one ISVD comprises three complementarity determining regions (CDR1 to CDR3, respectively), wherein:
   CDR1 comprises the amino acid sequence of SEQ ID NO: 270 (TADMG) (or has 3, 2 or 1 amino acid difference(s) with SEQ ID NO: 270);
   CDR2 comprises the amino acid sequence of SEQ ID NO: 271 (RISGIDGTTYYDEPVKG) (or has 3, 2 or 1 amino acid difference(s) with SEQ ID NO: 271); and
   CDR3 comprises the amino acid sequence of SEQ ID NO: 90 (PRYADQWSAYDY) (or has 3, 2 or 1 amino acid difference(s) with SEQ ID NO: 90);
wherein the CDR sequences are determined according to Kabat numbering.
45. The molecule according to any one of items 1-21, wherein the at least one ISVD comprises three complementarity determining regions (CDR1 to CDR3, respectively), wherein:
   CDR1 comprises the amino acid sequence of SEQ ID NO: 251 (or has 3, 2 or 1 amino acid difference(s) with SEQ ID NO: 251);
   CDR2 comprises the amino acid sequence of SEQ ID NO: 252 (or has 3, 2 or 1 amino acid difference(s) with SEQ ID NO: 252); and
   CDR3 comprises the amino acid sequence of SEQ ID NO: 253 (or has 3, 2 or 1 amino acid difference(s) with SEQ ID NO: 253);
wherein the CDR sequences are determined according to AbM definition.
46. The molecule according to any one of items 1-21, wherein the at least one ISVD comprises three complementarity determining regions (CDR1 to CDR3, respectively), wherein:
   CDR1 comprises the amino acid sequence of SEQ ID NO: 272 (SYGMG) (or has 3, 2 or 1 amino acid difference(s) with SEQ ID NO: 272);
   CDR2 comprises the amino acid sequence of SEQ ID NO: 273 (GISWRGDSTGYADSVRG) (or has 3, 2 or 1 amino acid difference(s) with SEQ ID NO: 273); and
   CDR3 comprises the amino acid sequence of SEQ ID NO: 253 (AAGSAWYGTLYEYDY) (or has 3, 2 or 1 amino acid difference(s) with SEQ ID NO: 253);
wherein the CDR sequences are determined according to Kabat numbering.
47. The molecule according to any one of items 1-21, wherein the at least one ISVD comprises three complementarity determining regions (CDR1 to CDR3, respectively), wherein:
   CDR1 comprises the amino acid sequence of SEQ ID NO: 272 (SYGMG) (or has 3, 2 or 1 amino acid difference(s) with SEQ ID NO: 272);
   CDR2 comprises the amino acid sequence of SEQ ID NO: 305 (GISWRGDSTGYADSVKG) (or has 3, 2 or 1 amino acid difference(s) with SEQ ID NO: 305); and
   CDR3 comprises the amino acid sequence of SEQ ID NO: 253 (AAGSAWYGTLYEYDY) (or has 3, 2 or 1 amino acid difference(s) with SEQ ID NO: 253);
wherein the CDR sequences are determined according to Kabat numbering.
48. The molecule according to any one of items 1-21, wherein the at least one ISVD comprises three complementarity determining regions (CDR1 to CDR3, respectively), wherein:
   CDR1 comprises the amino acid sequence of SEQ ID NO: 272 (SYGMG) (or has 3, 2 or 1 amino acid difference(s) with SEQ ID NO: 272);
   CDR2 comprises the amino acid sequence of SEQ ID NO: 306 (GISWRGDSTGYAKSVRG) (or has 3, 2 or 1 amino acid difference(s) with SEQ ID NO: 306); and
   CDR3 comprises the amino acid sequence of SEQ ID NO: 253 (AAGSAWYGTLYEYDY) (or has 3, 2 or 1 amino acid difference(s) with SEQ ID NO: 253);
wherein the CDR sequences are determined according to Kabat numbering.
49. The molecule according to any one of items 1-21, wherein the at least one ISVD comprises three complementarity determining regions (CDR1 to CDR3, respectively), wherein:
   CDR1 comprises the amino acid sequence of SEQ ID NO: 254 (GLTESTNPMY) (or has 3, 2 or 1 amino acid difference(s) with SEQ ID NO: 254);
   CDR2 comprises the amino acid sequence of SEQ ID NO: 255 (SISSRGITN) (or has 3, 2 or 1 amino acid difference(s) with SEQ ID NO: 255); and
   CDR3 comprises the amino acid sequence of SEQ ID NO: 256 (ASLSSGTVY) (or has 3, 2 or 1 amino acid difference(s) with SEQ ID NO: 256);
wherein the CDR sequences are determined according to AbM numbering.
50. The molecule according to any one of items 1-21, wherein the at least one ISVD comprises three complementarity determining regions (CDR1 to CDR3, respectively), wherein:
   CDR1 comprises the amino acid sequence of SEQ ID NO: 274 (TNPMY) (or has 3, 2 or 1 amino acid difference(s) with SEQ ID NO: 274);
   CDR2 comprises the amino acid sequence of SEQ ID NO: 275 (SISSRGITNYADSVRG) (or has 3, 2 or 1 amino acid difference(s) with SEQ ID NO: 275); and
   CDR3 comprises the amino acid sequence of SEQ ID NO: 256 (ASLSSGTVY) (or has 3, 2 or 1 amino acid difference(s) with SEQ ID NO: 256);
wherein the CDR sequences are determined according to Kabat numbering.
51. The molecule according to any one of items 1-21, wherein the at least one ISVD comprises three complementarity determining regions (CDR1 to CDR3, respectively), wherein:
   CDR1 comprises the amino acid sequence of SEQ ID NO: 274 (TNPMY) (or has 3, 2 or 1 amino acid difference(s) with SEQ ID NO: 274);
   CDR2 comprises the amino acid sequence of SEQ ID NO: 296 (SISSRGITNYADSVKG) (or has 3, 2 or 1 amino acid difference(s) with SEQ ID NO: 296); and
   CDR3 comprises the amino acid sequence of SEQ ID NO: 256 (ASLSSGTVY) (or has 3, 2 or 1 amino acid difference(s) with SEQ ID NO: 256);
wherein the CDR sequences are determined according to Kabat numbering.
52. The molecule according to any one of items 1-21, wherein the at least one ISVD comprises three complementarity determining regions (CDR1 to CDR3, respectively), wherein:
   CDR1 comprises the amino acid sequence of SEQ ID NO: 274 (TNPMY) (or has 3, 2 or 1 amino acid difference(s) with SEQ ID NO: 274);
   CDR2 comprises the amino acid sequence of SEQ ID NO: 297 (SISSRGITNYAKSVRG) (or has 3, 2 or 1 amino acid difference(s) with SEQ ID NO: 297); and
   CDR3 comprises the amino acid sequence of SEQ ID NO: 256 (ASLSSGTVY) (or has 3, 2 or 1 amino acid difference(s) with SEQ ID NO: 256);
wherein the CDR sequences are determined according to Kabat numbering.
53. The molecule according to any one of items 1-21, wherein the at least one ISVD comprises three complementarity determining regions (CDR1 to CDR3, respectively), wherein:
   CDR1 comprises the amino acid sequence of SEQ ID NO: 257 (GRTFSSYAMG) (or has 3, 2 or 1 amino acid difference(s) with SEQ ID NO: 257);
   CDR2 comprises the amino acid sequence of SEQ ID NO: 258 (AISWSDGSTY) (or has 3, 2 or 1 amino acid difference(s) with SEQ ID NO: 258); and
   CDR3 comprises the amino acid sequence of SEQ ID NO: 259 (DLTSTNPGSYIYIWAYDY) (or has 3, 2 or 1 amino acid difference(s) with SEQ ID NO: 259);
wherein the CDR sequences are determined according to AbM numbering.
54. The molecule according to any one of items 1-21, wherein the at least one ISVD comprises three complementarity determining regions (CDR1 to CDR3, respectively), wherein:
   CDR1 comprises the amino acid sequence of SEQ ID NO: 298 (SYAMG) (or has 3, 2 or 1 amino acid difference(s) with SEQ ID NO: 298);
   CDR2 comprises the amino acid sequence of SEQ ID NO: 299 (AISWSDGSTYYADSVRG) (or has 3, 2 or 1 amino acid difference(s) with SEQ ID NO: 299); and
   CDR3 comprises the amino acid sequence of SEQ ID NO: 259 (DLTSTNPGSYIYIWAYDY) (or has 3, 2 or 1 amino acid difference(s) with SEQ ID NO: 259);
wherein the CDR sequences are determined according to Kabat numbering.
55. The molecule according to any one of items 1-21, wherein the at least one ISVD comprises three complementarity determining regions (CDR1 to CDR3, respectively), wherein:
   CDR1 comprises the amino acid sequence of SEQ ID NO: 298 (SYAMG) (or has 3, 2 or 1 amino acid difference(s) with SEQ ID NO: 298);
   CDR2 comprises the amino acid sequence of SEQ ID NO: 230 (AISWSDGSTYYADSVKG) (or has 3, 2 or 1 amino acid difference(s) with SEQ ID NO: 230); and
   CDR3 comprises the amino acid sequence of SEQ ID NO: 259 (DLTSTNPGSYIYIWAYDY) (or has 3, 2 or 1 amino acid difference(s) with SEQ ID NO: 259);
wherein the CDR sequences are determined according to Kabat numbering.
56. The molecule according to any one of items 1-21, wherein the at least one ISVD comprises three complementarity determining regions (CDR1 to CDR3, respectively), wherein:
   CDR1 comprises the amino acid sequence of SEQ ID NO: 261 (GFTFEDYAIG) (or has 3, 2 or 1 amino acid difference(s) with SEQ ID NO: 261);
   CDR2 comprises the amino acid sequence of SEQ ID NO: 262 (CIRTYDEQTY) (or has 3, 2 or 1 amino acid difference(s) with SEQ ID NO: 262); and
   CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to AbM numbering.
57. The molecule according to any one of items 1-21, wherein the at least one ISVD comprises three complementarity determining regions (CDR1 to CDR3, respectively), wherein:
   CDR1 comprises the amino acid sequence of SEQ ID NO: 261 (GFTFEDYAIG) (or has 3, 2 or 1 amino acid difference(s) with SEQ ID NO: 261);
   CDR2 comprises the amino acid sequence of SEQ ID NO: 262 (CIRTYDEQTY) (or has 3, 2 or 1 amino acid difference(s) with SEQ ID NO: 262); and
   CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to AbM numbering.
58. The molecule according to any one of items 1-21, wherein the at least one ISVD comprises three complementarity determining regions (CDR1 to CDR3, respectively), wherein:
   CDR1 comprises the amino acid sequence of SEQ ID NO: 301 (DYAIG) (or has 3, 2 or 1 amino acid difference(s) with SEQ ID NO: 301);
   CDR2 comprises the amino acid sequence of SEQ ID NO: 302 (CIRTYDEQTYYADSVKG) (or has 3, 2 or 1 amino acid difference(s) with SEQ ID NO: 302); and
   CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to Kabat numbering.
59. The molecule according to any one of items 1-21, wherein the at least one ISVD comprises three complementarity determining regions (CDR1 to CDR3, respectively), wherein:
   CDR1 comprises the amino acid sequence of SEQ ID NO: 301 (DYAIG) (or has 3, 2 or 1 amino acid difference(s) with SEQ ID NO: 301);
   CDR2 comprises the amino acid sequence of SEQ ID NO: 303 (CIRTYDEQTYYADSVRG) (or has 3, 2 or 1 amino acid difference(s) with SEQ ID NO: 303); and
   CDR3 comprises the amino acid sequence of SEQ ID NO: 263 (GSYYACAYYSRPDPSEGHVDLDY) (or has 3, 2 or 1 amino acid difference(s) with SEQ ID NO: 263);
wherein the CDR sequences are determined according to Kabat numbering.
60. The molecule according to any one of items 1-59, wherein the at least one ISVD is derived from SEQ ID NOs.: 1, 26, 43, 68, 225-227, 250, preferably wherein the at least one ISVD is selected from the ISVDs with SEQ ID NOs: 1, 3-26, 28-43, 45-75, 78 and 173-250.
61. The molecule according to any one of items 1-60, wherein the reactive group in the side chain of the one or more lysines comprised in the at least one ISVD is an attachment point or conjugation site which allows for conjugation of a cargo to the ISVD.
62. The molecule according to any one of items 1-61, wherein the at least one ISVD comprises at least one cargo attached to at least one of the attachment points or conjugation sites.
63. The molecule according to item 62, wherein the at least one cargo is directly attached to the side chain of the lysine of the at least one ISVD or wherein the at least one cargo is attached to the side chain of the lysine of the at least one ISVD through a linker.
64. The molecule according to item 63, wherein the linker is a cleavable linker.
65. The molecule according to any one of items 63 or 64, wherein the linker is a peptide linker.
66. The molecule according to any one of items 63 or 64, wherein the linker is not a peptide linker.
67. The molecule according to any one of items 63 to 65, wherein the linker is an amino acid or an amino acid sequence, preferably of between 1 and 50, such as for example Gly-Ser linkers ((glyₓser_{y})_{z}), or A3, GS30, GS15, GS9 and GS7 linkers, or a linker as defined in SEQ ID NOs.: 107-123, more preferably a linker as defined in SEQ ID NO.: 113.
68. The molecule according to any one of items 63, 64 or 66, wherein the linker is a linear or branched polyethylene glycol (PEG) moiety, preferably with a molecular weight of about 1-60 kDa, preferably with a weight of about 1-10 kDa, such as 5 kDa or 10 kDa.
69. The molecule according to any one of items 63 to 65 or 67, wherein the linker is an ELNN polypeptide.
70. The molecule according to any one of items 63, 64 or 66, wherein the linker is an APN-maleimide linker (3-(4-(2,5-dioxo-2,5-dihydro-1H-pyrrol-1-yl)phenyl) propiolonitrile, MAPN) or a bis-maleimido-PEG3 (BM (PEG)₃) linker (BM (PEG)₃ (1,11-bismaleimido-triethyleneglycol)).
71. The molecule according to any one of items 62 to 70, wherein the at least one cargo is directly attached to the side chain of the lysine of the at least one ISVD or wherein the at least one cargo is attached to the side chain of the lysine of the at least one ISVD through a linker via an amide bond.
72. The molecule according to any one of items 1 to 71, wherein the molecule comprises at least one ISVD and at least one further moiety or cargo, preferably wherein the at least one further moiety or cargo is selected from
   a) a half-life extending (HLE) moiety, and/or
   b) a targeting moiety; and/or
   c) a therapeutic moiety or precursor therefrom;
   d) an imaging moiety;
   e) a toxic moiety;
   f) siRNA;
   g) vitamins; and/or
   h) Toll-like receptor agonists.

wherein the cargo is attached to the side chain of the lysine of the at least one ISVD.
73. The molecule according to any one of items 1 to 72, wherein the cargo is an (in vivo) half-life extending moiety, preferably a PEG molecule, an ELNN polypeptide or an albumin-binding polypeptide.
74. A nucleic acid encoding the molecule as defined in any one of items 1 to 73 and/or part of the molecule as defined in any one of items 1-73.
75. A vector comprising the nucleic acid as defined in item 74.
76. A composition comprising the molecule as defined in any one of items 1 to 73, such as a pharmaceutical composition.
77. A method for producing the molecule of any of items 1 to 73, comprising
   a) providing a cargo, wherein the cargo comprises at least one reactive group; and
   b) conjugating the cargo provided in step a. to at least one lysine comprised in the at least one ISVD comprised in the molecule as defined in any of items 1 to 73, preferably via the formation of an amide bond.
78. The method according to item 77, wherein the reactive group present in the cargo is an ester, preferably a NHS ester.
79. A method for producing the molecule as defined in any one of items 1 to 73, wherein the method comprises:
   a) expressing, in a suitable host cell or host organism or in another suitable expression system, a nucleic acid sequence encoding the at least one ISVD and/or the molecule or part of the molecule as defined in any one of items 1 to 73;
   b) optionally isolating and/or purifying the at least one ISVD and/or the molecule or part of the molecule expressed in a);
   c) optionally conjugating one or more (further) cargos to the attachment point(s) or conjugation sites(s) of the at least one ISVD, wherein the attachment point(s) or conjugation sites(s) is (are) primary amine(s) in the side chain of lysine(s) located at solvent accessible positions, or at the C-terminus, or the N-terminal primary amine.
80. A method for producing the molecule as defined in any one of items 1 to 73, wherein the method comprises:
   a) chemically synthesizing the at least one ISVD and/or the molecule or part of the molecule as defined in any one of items 1 to 73, preferably by using solid-phase peptide synthesis;
   b) optionally isolating and/or purifying the at least one ISVD and/or the molecule or part of the molecule synthesized in a);
   c) optionally conjugating one or more (further) cargos to the attachment point(s) or conjugation sites(s) of the ISVD, wherein the attachment point(s) or conjugation sites(s) is (are) primary amine(s) in the side chain of lysine(s) located at solvent accessible positions, or at the C-terminus, or the N-terminal primary amine.
81. A method for producing an immunoglobulin single variable domain (ISVD) suitable for lysine site-specific conjugation comprising the steps of:
   a. Providing an ISVD that does not comprise any lysines in its sequence;
   b. Substituting at least one amino acid present at a solvent accessible position in the ISVD provided in step a. by a lysine, to generate an ISVD suitable for lysine site-specific conjugation.
82. The method according to claim 81, wherein the ISVD comprises an amino acid selected from glutamine, histidine, arginine, alanine, and glutamate, preferably arginine at one or more, or preferably all, of positions 43, 64 and 75 (according to Kabat).
83. The method according to claim 81, wherein the ISVD comprises an amino acid selected from glutamine, histidine, arginine, and glutamate, preferably arginine at one or more, or preferably all, of positions 43, 64 and 75 (according to Kabat).
84. The method according to claim 83, wherein the ISVD comprises an amino acid selected from glutamine, histidine, arginine, alanine, and glutamate, preferably arginine at one or more, or preferably all, of positions 43, 64, 75 and 83 (according to Kabat).
85. The method according to claim 83, wherein the ISVD comprises an amino acid selected from glutamine, histidine, arginine, and glutamate, preferably arginine at one or more, or preferably all, of positions 43, 64, 75 and 83 (according to Kabat).
86. A method for producing an immunoglobulin single variable domain (ISVD) suitable for primary amine site-specific conjugation comprising the steps of:
   a. Providing an ISVD, preferably wherein the ISVD comprises at least one lysine in its amino acid sequence, preferably at a solvent accessible position, and optionally wherein the N-terminus of the ISVD comprises a pyroglutamate modification;
   b. Substituting the at least one lysing comprised in the ISVD provided in step a. by another amino acid, preferably selected from glutamine, histidine, arginine, alanine, or glutamate, more preferably by arginine, to generate an ISVD with no lysines in its sequence;
   c. Substituting at least one amino acid present at a solvent accessible position in the ISVD generated in step b. by a lysine, to generate an ISVD suitable for site-specific conjugation.
87. A method for producing an immunoglobulin single variable domain (ISVD) that does not comprise any lysines in its amino acid sequence, comprising the steps of:
   a. Providing an ISVD, preferably wherein the ISVD comprises at least one lysine in its amino acid sequence, preferably at a solvent accessible position;
   b. Substituting the at least one lysing by another amino acid, preferably selected from glutamine, histidine, arginine, alanine, or glutamate, more preferably by arginine, to generate an ISVD that does not comprise any lysines in its amino acid sequence.
88. The method according to item 87, wherein the ISVD provided in stap a. has a free N-terminus (comprises a primary amine in its N-terminus).
89. The method according to any one of items 81 to 86, wherein the solvent accessible position are selected from the following positions: 5, 7, 10, 12, 13, 19, 43, 44, 45, 61, 64, 70, 71, 73, 75, 83, 85, 105 and/or 108 (according to Kabat), preferably from the following positions: 19, 43, 45, 64, 71, 73, 75, 83 and/or 105, such as at positions 43 and/or 45.
90. An immunoglobulin single variable domain (ISVD) directly produced by the method of any one of items 81-89.
91. The molecule according to any one of items 1 to 73, or produced by the method according to any one of items 77-79, the immunoglobulin single variable domain produced by the method according to any one of items 81-90, or the composition according to item 76 for use in medicine.
92. The molecule according to any one of items 1 to 73, or produced by the method according to any one of items 77-79, the immunoglobulin single variable domain produced by the method according to any one of items 81-90, or the composition according to item 76 for use in the prophylactic and/or therapeutic treatment of an autoimmune/inflammatory disease, an infectious disease and/or proliferative disease/cancer, such as hematological (blood) and solid tumor cancer disease.
93. The molecule according to any one of items 1 to 73, or produced by the method according to any one of items 77-79, the immunoglobulin single variable domain produced by the method according to any one of items 81-90, or the composition according to item 76 for use as a vaccine.
94. A vaccine comprising a molecule as defined in any one of items 1 to 73, or produced by the method according to any one of items 36-377-799, the immunoglobulin single variable domain according to any one of items 81-90, or the composition as defined in item 76, optionally further comprising an adjuvant.

1. A molecule comprising or consisting of at least one immunoglobulin single variable domain (ISVD), wherein the at least one ISVD:
comprises at least one engineered lysine in its amino acid sequence, wherein the ISVD comprises an engineered lysine at at least one solvent accessible position, wherein the ISVD does not comprise an engineered lysine at any of positions 19, 43, 64, 75, 83, 97 and 105 (according to Kabat); or
comprises only two lysines in its amino acid sequence, wherein the two lysines are located at two positions selected from the group consisting of positions 43, 64, 75 and 83 (according to Kabat); or
does not comprise any primary amine in its sequence.

2. The molecule of claim 1, wherein the at least one ISVD is suitable for site-specific conjugation on at least one primary amine present on the side chain of a lysine and/or in an N-terminal primary amine.

3. The molecule according to claim 1, wherein the at least one ISVD comprises more than one lysine.

4. The molecule according to claim 1, wherein the at least one ISVD comprises lysine at at least one of the following positions: 5, 7, 10, 12, 13, 19, 43, 44, 45, 64, 61, 70, 71, 73, 75, 83, 85, 105 and/or 108 (according to Kabat).

5. The molecule according to claim 1, wherein the at least one ISDV comprises a glutamine at position 1 (according to Kabat), optionally wherein the N-terminus of the at least one ISVD comprises a pyroglutamate modification.

6. The molecule according to claim 1, wherein the at least one ISVD comprises or consists of three complementarity determining regions (CDR1 to CDR3, respectively), wherein:
CDR1 comprises the amino acid sequence of SEQ ID NO: 79 or an amino acid sequence that has 3, 2 or 1 amino acid difference(s) with SEQ ID NO: 79;
CDR2 comprises the amino acid sequence of SEQ ID NO: 80 or an amino acid sequence that has 3, 2 or 1 amino acid difference(s) with SEQ ID NO: 80; and
CDR3 comprises the amino acid sequence of SEQ ID NO: 81 or an amino acid sequence that has 3, 2 or 1 amino acid difference(s) with SEQ ID NO: 81);
wherein the CDR sequences are determined according to AbM numbering.

7. The molecule according to claim 1, wherein the at least one ISVD comprises three complementarity determining regions (CDR1 to CDR3, respectively), wherein:
CDR1 comprises the amino acid sequence of SEQ ID NO: 82 or an amino acid sequence that has 3, 2 or 1 amino acid difference(s) with SEQ ID NO: 82;
CDR2 comprises the amino acid sequence of SEQ ID NO: 83 or an amino acid sequence that has 3, 2 or 1 amino acid difference(s) with SEQ ID NO: 83; and
CDR3 comprises the amino acid sequence of SEQ ID NO: 84 or an amino acid sequence that has 3, 2 or 1 amino acid difference(s) with SEQ ID NO: 84;
wherein the CDR sequences are determined according to AbM numbering.

8. The molecule according to claim 1, wherein the at least one ISVD comprises three complementarity determining regions (CDR1 to CDR3, respectively), wherein:
CDR1 comprises the amino acid sequence of SEQ ID NO: 85 or an amino acid sequence that has 3, 2 or 1 amino acid difference(s) with SEQ ID NO: 85;
CDR2 comprises the amino acid sequence of SEQ ID NO: 86 or an amino acid sequence that has 3, 2 or 1 amino acid difference(s) with SEQ ID NO: 86; and
CDR3 comprises the amino acid sequence of SEQ ID NO: 87 or an amino acid sequence that has 3, 2 or 1 amino acid difference(s) with SEQ ID NO: 87;
wherein the CDR sequences are determined according to AbM numbering.

9. The molecule according to claim 1, wherein the at least one ISVD comprises three complementarity determining regions (CDR1 to CDR3, respectively), wherein:
CDR1 comprises the amino acid sequence of SEQ ID NO: 88 or an amino acid sequence that has 3, 2 or 1 amino acid difference(s) with SEQ ID NO: 88;
CDR2 comprises the amino acid sequence of SEQ ID NO: 89 or an amino acid sequence that has 3, 2 or 1 amino acid difference(s) with SEQ ID NO: 89; and
CDR3 comprises the amino acid sequence of SEQ ID NO: 90 or an amino acid sequence that has 3, 2 or 1 amino acid difference(s) with SEQ ID NO: 90;
wherein the CDR sequences are determined according to AbM numbering.

10. The molecule according to claim 1, wherein the at least one ISVD comprises three complementarity determining regions (CDR1 to CDR3, respectively), wherein:
CDR1 comprises the amino acid sequence of SEQ ID NO: 251 or an amino acid sequence that has 3, 2 or 1 amino acid difference(s) with SEQ ID NO: 251;
CDR2 comprises the amino acid sequence of SEQ ID NO: 252 or an amino acid sequence that has 3, 2 or 1 amino acid difference(s) with SEQ ID NO: 252; and
CDR3 comprises the amino acid sequence of SEQ ID NO: 253 or an amino acid sequence that has 3, 2 or 1 amino acid difference(s) with SEQ ID NO: 253;
wherein the CDR sequences are determined according to AbM definition.

11. The molecule according to claim 1, wherein the at least one ISVD comprises three complementarity determining regions (CDR1 to CDR3, respectively), wherein:
CDR1 comprises the amino acid sequence of SEQ ID NO: 254 or an amino acid sequence that has 3, 2 or 1 amino acid difference(s) with SEQ ID NO: 254;
CDR2 comprises the amino acid sequence of SEQ ID NO: 255 or an amino acid sequence that has 3, 2 or 1 amino acid difference(s) with SEQ ID NO: 255; and
CDR3 comprises the amino acid sequence of SEQ ID NO: 256 or an amino acid sequence that has 3, 2 or 1 amino acid difference(s) with SEQ ID NO: 256;
wherein the CDR sequences are determined according to AbM numbering.

12. The molecule according to claim 1, wherein the at least one ISVD comprises three complementarity determining regions (CDR1 to CDR3, respectively), wherein:
CDR1 comprises the amino acid sequence of SEQ ID NO: 257 or an amino acid sequence that has 3, 2 or 1 amino acid difference(s) with SEQ ID NO: 257;
CDR2 comprises the amino acid sequence of SEQ ID NO: 258 or an amino acid sequence that has 3, 2 or 1 amino acid difference(s) with SEQ ID NO: 258; and
CDR3 comprises the amino acid sequence of SEQ ID NO: 259 or an amino acid sequence that has 3, 2 or 1 amino acid difference(s) with SEQ ID NO: 259;
wherein the CDR sequences are determined according to AbM numbering.

13. The molecule according to claim 1, wherein the at least one ISVD comprises three complementarity determining regions (CDR1 to CDR3, respectively), wherein:
CDR1 comprises the amino acid sequence of SEQ ID NO: 261 or an amino acid sequence that has 3, 2 or 1 amino acid difference(s) with SEQ ID NO: 261;
CDR2 comprises the amino acid sequence of SEQ ID NO: 262 or an amino acid sequence that has 3, 2 or 1 amino acid difference(s) with SEQ ID NO: 262; and
CDR3 comprises the amino acid sequence of SEQ ID NO: 263 or an amino acid sequence that has 3, 2 or 1 amino acid difference(s) with SEQ ID NO: 263;
wherein the CDR sequences are determined according to AbM numbering.

14. The molecule according to claim 1, wherein the at least one ISVD comprising an amino acid sequence of any one of SEQ ID NOs: 1, 3-26, 28-43, 45-75, 78 and 173-250.

15. The molecule according to claim 1, wherein the at least one ISVD comprises at least one cargo attached to a primary amine present on the side chain of a lysine, optionally wherein the at least one cargo comprises:
a) a half-life extending (HLE) moiety, and/or;
b) a targeting moiety; and/or
c) a therapeutic moiety or precursor therefrom;
d) an imaging moiety;
e) a toxic moiety;
f) an siRNA;
g) vitamins; and/or
h) a toll-like receptor agonist.

16. A method for producing an immunoglobulin single variable domain (ISVD) suitable for lysine site-specific conjugation comprising the steps of:
a. providing an ISVD with no lysines in its sequence, and optionally wherein the N-terminus of the ISVD comprises a pyroglutamate modification; and
b. substituting at least one amino acid present at a solvent accessible position in the ISVD provided in step a. by a lysine, to generate an ISVD suitable for lysine site-specific conjugation.

17. The method according to claim 16, wherein step a. comprises:
i. providing an ISVD, wherein the ISVD comprises at least one lysine in its amino acid sequence, optionally wherein the ISVD comprises a lysine at a solvent accessible position;
ii. substituting the at least one lysine comprised in the ISVD provided in step i. by another amino acid selected from glutamine, histidine, arginine, alanine, or glutamate, to generate the ISVD with no lysines in its sequence.

18. The method according to claim 16, wherein the solvent accessible position is at any one of positions: 5, 7, 10, 12, 13, 19, 43, 44, 45, 64, 61, 70, 71, 73, 75, 83, 85, 105 and 108 (according to Kabat).

19. A method of treatment comprising administering the molecule according to claim 1 to a subject in need thereof.

20. A nucleic acid encoding a polypeptide comprising or consisting of at least one immunoglobulin single variable domain (ISVD), wherein the at least one ISVD:
comprises at least one engineered lysine in its amino acid sequence, wherein the ISVD comprises an engineered lysine at at least one solvent accessible position, wherein the ISVD does not comprise an engineered lysine at any of positions 19, 43, 64, 75, 83, 97 or 105 (according to Kabat); or
comprises only two lysines in its amino acid sequence, wherein the two lysines are located at two positions selected from the group consisting of positions 43, 64, 75 and/or 83 (according to Kabat); or
does not comprise any primary amine in its sequence.


SEQUENCE LISTING (306 sequences)

SEQ ID NO: 1 - protein, 124 aa, synthetic construct
EVQLVESGGGVVQPGGSLRLSCAASGLTFSTYTMGWFRQAPGKEREFVAAIIWSGSNTYYADSVKGRFTISRDNAKNTVYLQMNSLRPEDTALYYCAAQHFGPIGLTTRGYHYWGQGTLVTVSS

SEQ ID NO: 2 - skipped

SEQ ID NO: 3 - protein, 124 aa, synthetic construct
EVQLVESGGGVVQPGGSLRLSCAASGLTFSTYTMGWFRQAPGEEREFVAAIIWSGSNTYYADSVKGRFTISRDNAKNTVYLQMNSLRPEDTALYYCAAQHFGPIGLTTRGYHYWGQGTLVTVSS

SEQ ID NO: 4 - protein, 124 aa, synthetic construct
EVQLVESGGGVVQPGGSLRLSCAASGLTFSTYTMGWFRQAPGQEREFVAAIIWSGSNTYYADSVKGRFTISRDNAKNTVYLQMNSLRPEDTALYYCAAQHFGPIGLTTRGYHYWGQGTLVTVSS

SEQ ID NO: 5 - protein, 124 aa, synthetic construct
EVQLVESGGGVVQPGGSLRLSCAASGLTFSTYTMGWFRQAPGREREFVAAIIWSGSNTYYADSVKGRFTISRDNAKNTVYLQMNSLRPEDTALYYCAAQHFGPIGLTTRGYHYWGQGTLVTVSS

SEQ ID NO: 6 - protein, 124 aa, synthetic construct
EVQLVESGGGVVQPGGSLRLSCAASGLTFSTYTMGWFRQAPGHEREFVAAIIWSGSNTYYADSVKGRFTISRDNAKNTVYLQMNSLRPEDTALYYCAAQHFGPIGLTTRGYHYWGQGTLVTVSS

SEQ ID NO: 7 - protein, 124 aa, synthetic construct
EVQLVESGGGVVQPGGSLRLSCAASGLTFSTYTMGWFRQAPGAEREFVAAIIWSGSNTYYADSVKGRFTISRDNAKNTVYLQMNSLRPEDTALYYCAAQHFGPIGLTTRGYHYWGQGTLVTVSS

SEQ ID NO: 8 - protein, 124 aa, synthetic construct
EVQLVESGGGVVQPGGSLRLSCAASGLTFSTYTMGWFRQAPGKEREFVAAIIWSGSNTYYADSVEGRFTISRDNAKNTVYLQMNSLRPEDTALYYCAAQHFGPIGLTTRGYHYWGQGTLVTVSS

SEQ ID NO: 9 - protein, 124 aa, synthetic construct
EVQLVESGGGVVQPGGSLRLSCAASGLTFSTYTMGWFRQAPGKEREFVAAIIWSGSNTYYADSVQGRFTISRDNAKNTVYLQMNSLRPEDTALYYCAAQHFGPIGLTTRGYHYWGQGTLVTVSS

SEQ ID NO: 10 - protein, 124 aa, synthetic construct
EVQLVESGGGVVQPGGSLRLSCAASGLTFSTYTMGWFRQAPGKEREFVAAIIWSGSNTYYADSVRGRFTISRDNAKNTVYLQMNSLRPEDTALYYCAAQHFGPIGLTTRGYHYWGQGTLVTVSS

SEQ ID NO: 11 - protein, 124 aa, synthetic construct
EVQLVESGGGVVQPGGSLRLSCAASGLTFSTYTMGWFRQAPGKEREFVAAIIWSGSNTYYADSVHGRFTISRDNAKNTVYLQMNSLRPEDTALYYCAAQHFGPIGLTTRGYHYWGQGTLVTVSS

SEQ ID NO: 12 - protein, 124 aa, synthetic construct
EVQLVESGGGVVQPGGSLRLSCAASGLTFSTYTMGWFRQAPGKEREFVAAIIWSGSNTYYADSVAGRFTISRDNAKNTVYLQMNSLRPEDTALYYCAAQHFGPIGLTTRGYHYWGQGTLVTVSS

SEQ ID NO: 13 - protein, 124 aa, synthetic construct
EVQLVESGGGVVQPGGSLRLSCAASGLTFSTYTMGWFRQAPGKEREFVAAIIWSGSNTYYADSVKGRFTISRDNAENTVYLQMNSLRPEDTALYYCAAQHFGPIGLTTRGYHYWGQGTLVTVSS

SEQ ID NO: 14 - protein, 124 aa, synthetic construct
EVQLVESGGGVVQPGGSLRLSCAASGLTFSTYTMGWFRQAPGKEREFVAAIIWSGSNTYYADSVKGRFTISRDNAQNTVYLQMNSLRPEDTALYYCAAQHFGPIGLTTRGYHYWGQGTLVTVSS

SEQ ID NO: 15 - protein, 124 aa, synthetic construct
EVQLVESGGGVVQPGGSLRLSCAASGLTFSTYTMGWFRQAPGKEREFVAAIIWSGSNTYYADSVKGRFTISRDNARNTVYLQMNSLRPEDTALYYCAAQHFGPIGLTTRGYHYWGQGTLVTVSS

SEQ ID NO: 16 - protein, 124 aa, synthetic construct
EVQLVESGGGVVQPGGSLRLSCAASGLTFSTYTMGWFRQAPGKEREFVAAIIWSGSNTYYADSVKGRFTISRDNAHNTVYLQMNSLRPEDTALYYCAAQHFGPIGLTTRGYHYWGQGTLVTVSS

SEQ ID NO: 17 - protein, 124 aa, synthetic construct
EVQLVESGGGVVQPGGSLRLSCAASGLTFSTYTMGWFRQAPGKEREFVAAIIWSGSNTYYADSVKGRFTISRDNAANTVYLQMNSLRPEDTALYYCAAQHFGPIGLTTRGYHYWGQGTLVTVSS

SEQ ID NO: 18 - protein, 124 aa, synthetic construct
EVQLVESGGGVVQPGGSLRLSCAASGLTFSTYTMGWFRQAPGREREFVAAIIWSGSNTYYADSVKGRFTISRDNARNTVYLQMNSLRPEDTALYYCAAQHFGPIGLTTRGYHYWGQGTLVTVSS

SEQ ID NO: 19 - protein, 124 aa, synthetic construct
EVQLVESGGGVVQPGGSLRLSCAASGLTFSTYTMGWFRQAPGREKEFVAAIIWSGSNTYYADSVRGRFTISRDNARNTVYLQMNSLRPEDTALYYCAAQHFGPIGLTTRGYHYWGQGTLVTVSS

SEQ ID NO: 20 - protein, 124 aa, synthetic construct
EVQLVESGGGVVQPGGSLRLSCAASGLTFSTYTMGWFRQAPGREREFVAAIIWSGSNTYYADSVRGRFTISRDNARNTVYLQMNSLRPEDTALYYCAAQHFGPIGLTTRGYHYWGKGTLVTVSS

SEQ ID NO: 21 - protein, 124 aa, synthetic construct
EVQLVESGGGVVQPGGSLRLSCAASGLTFSTYTMGWFRQAPGREREFVAAIIWSGSNTYYADSVRGRFTISRDNARNTVYLQMNSLRPEDTALYYCAAQHFGPIGLTTRGYHYWGQGTLVTVSS

SEQ ID NO: 22 - protein, 124 aa, synthetic construct
EVQLVESGGGVVQPGGSLRLSCAASGLTFSTYTMGWFRQAPGREREFVAAIIWSGSNTYYADSVRGRFTISRDNAKNTVYLQMNSLRPEDTALYYCAAQHFGPIGLTTRGYHYWGQGTLVTVSS

SEQ ID NO: 23 - protein, 124 aa, synthetic construct
EVQLVESGGGVVQPGGSLRLSCAASGLTFSTYTMGWFRQAPGREREFVAAIIWSGSNTYYADSVRGRFTISRDKARNTVYLQMNSLRPEDTALYYCAAQHFGPIGLTTRGYHYWGQGTLVTVSS

SEQ ID NO: 24 - protein, 124 aa, synthetic construct
EVQLVESGGGVVQPGGSLRLSCAASGLTFSTYTMGWFRQAPGKEREFVAAIIWSGSNTYYADSVRGRFTISRDNARNTVYLQMNSLRPEDTALYYCAAQHFGPIGLTTRGYHYWGQGTLVTVSS

SEQ ID NO: 25 - protein, 125 aa, synthetic construct
EVQLVESGGGVVQPGGSLRLSCAASGLTFSTYTMGWFRQAPGREREFVAAIIWSGSNTYYADSVRGRFTISRDNARNTVYLQMNSLRPEDTALYYCAAQHFGPIGLTTRGYHYWGQGTLVTVSSK

SEQ ID NO: 26 - protein, 123 aa, synthetic construct
EVQLVESGGGVVQPGGSLRLSCAASGHTFSEYALGWFRQAPGKEREFVAAINWGGGWTYYADSVKGRFTISRDNAKNTLYLQMNSLRPEDTALYYCAASSDYAGGNPTGYPYWGQGTLVTVSS

SEQ ID NO: 27 - skipped

SEQ ID NO: 28 - protein, 123 aa, synthetic construct
EVQLVESGGGVVQPGGSLRLSCAASGHTFSEYALGWFRQAPGEEREFVAAINWGGGWTYYADSVKGRFTISRDNAKNTLYLQMNSLRPEDTALYYCAASSDYAGGNPTGYPYWGQGTLVTVSS

SEQ ID NO: 29 - protein, 123 aa, synthetic construct
EVQLVESGGGVVQPGGSLRLSCAASGHTFSEYALGWFRQAPGQEREFVAAINWGGGWTYYADSVKGRFTISRDNAKNTLYLQMNSLRPEDTALYYCAASSDYAGGNPTGYPYWGQGTLVTVSS

SEQ ID NO: 30 - protein, 123 aa, synthetic construct
EVQLVESGGGVVQPGGSLRLSCAASGHTFSEYALGWFRQAPGREREFVAAINWGGGWTYYADSVKGRFTISRDNAKNTLYLQMNSLRPEDTALYYCAASSDYAGGNPTGYPYWGQGTLVTVSS

SEQ ID NO: 31 - protein, 123 aa, synthetic construct
EVQLVESGGGVVQPGGSLRLSCAASGHTFSEYALGWFRQAPGHEREFVAAINWGGGWTYYADSVKGRFTISRDNAKNTLYLQMNSLRPEDTALYYCAASSDYAGGNPTGYPYWGQGTLVTVSS

SEQ ID NO: 32 - protein, 123 aa, synthetic construct
EVQLVESGGGVVQPGGSLRLSCAASGHTFSEYALGWFRQAPGAEREFVAAINWGGGWTYYADSVKGRFTISRDNAKNTLYLQMNSLRPEDTALYYCAASSDYAGGNPTGYPYWGQGTLVTVSS

SEQ ID NO: 33 - protein, 123 aa, synthetic construct
EVQLVESGGGVVQPGGSLRLSCAASGHTFSEYALGWFRQAPGKEREFVAAINWGGGWTYYADSVEGRFTISRDNAKNTLYLQMNSLRPEDTALYYCAASSDYAGGNPTGYPYWGQGTLVTVSS

SEQ ID NO: 34 - protein, 123 aa, synthetic construct
EVQLVESGGGVVQPGGSLRLSCAASGHTFSEYALGWFRQAPGKEREFVAAINWGGGWTYYADSVQGRFTISRDNAKNTLYLQMNSLRPEDTALYYCAASSDYAGGNPTGYPYWGQGTLVTVSS

SEQ ID NO: 35 - protein, 123 aa, synthetic construct
EVQLVESGGGVVQPGGSLRLSCAASGHTFSEYALGWFRQAPGKEREFVAAINWGGGWTYYADSVRGRFTISRDNAKNTLYLQMNSLRPEDTALYYCAASSDYAGGNPTGYPYWGQGTLVTVSS

SEQ ID NO: 36 - protein, 123 aa, synthetic construct
EVQLVESGGGVVQPGGSLRLSCAASGHTFSEYALGWFRQAPGKEREFVAAINWGGGWTYYADSVHGRFTISRDNAKNTLYLQMNSLRPEDTALYYCAASSDYAGGNPTGYPYWGQGTLVTVSS

SEQ ID NO: 37 - protein, 123 aa, synthetic construct
EVQLVESGGGVVQPGGSLRLSCAASGHTFSEYALGWFRQAPGKEREFVAAINWGGGWTYYADSVAGRFTISRDNAKNTLYLQMNSLRPEDTALYYCAASSDYAGGNPTGYPYWGQGTLVTVSS

SEQ ID NO: 38 - protein, 123 aa, synthetic construct
EVQLVESGGGVVQPGGSLRLSCAASGHTFSEYALGWFRQAPGKEREFVAAINWGGGWTYYADSVKGRFTISRDNAENTLYLQMNSLRPEDTALYYCAASSDYAGGNPTGYPYWGQGTLVTVSS

SEQ ID NO: 39 - protein, 123 aa, synthetic construct
EVQLVESGGGVVQPGGSLRLSCAASGHTFSEYALGWFRQAPGKEREFVAAINWGGGWTYYADSVKGRFTISRDNAQNTLYLQMNSLRPEDTALYYCAASSDYAGGNPTGYPYWGQGTLVTVSS

SEQ ID NO: 40 - protein, 123 aa, synthetic construct
EVQLVESGGGVVQPGGSLRLSCAASGHTFSEYALGWFRQAPGKEREFVAAINWGGGWTYYADSVKGRFTISRDNARNTLYLQMNSLRPEDTALYYCAASSDYAGGNPTGYPYWGQGTLVTVSS

SEQ ID NO: 41 - protein, 123 aa, synthetic construct
EVQLVESGGGVVQPGGSLRLSCAASGHTFSEYALGWFRQAPGKEREFVAAINWGGGWTYYADSVKGRFTISRDNAHNTLYLQMNSLRPEDTALYYCAASSDYAGGNPTGYPYWGQGTLVTVSS

SEQ ID NO: 42 - protein, 123 aa, synthetic construct
EVQLVESGGGVVQPGGSLRLSCAASGHTFSEYALGWFRQAPGKEREFVAAINWGGGWTYYADSVKGRFTISRDNAANTLYLQMNSLRPEDTALYYCAASSDYAGGNPTGYPYWGQGTLVTVSS

SEQ ID NO: 43 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 44 - skipped

SEQ ID NO: 45 - protein, 115 aa, synthetic construct
EVQLVESGGGVVQPGGSLRLSCAASGFTFRSFGMSWVRQAPGEGPEWVSSISGSGSDTLYADSVKGRFTISRDNSKNTLYLQMNSLRPEDTALYYCTIGGSLSRSSQGTLVTVSS

SEQ ID NO: 46 - protein, 115 aa, synthetic construct
EVQLVESGGGVVQPGGSLRLSCAASGFTFRSFGMSWVRQAPGQGPEWVSSISGSGSDTLYADSVKGRFTISRDNSKNTLYLQMNSLRPEDTALYYCTIGGSLSRSSQGTLVTVSS

SEQ ID NO: 47 - protein, 115 aa, synthetic construct
EVQLVESGGGVVQPGGSLRLSCAASGFTFRSFGMSWVRQAPGHGPEWVSSISGSGSDTLYADSVKGRFTISRDNSKNTLYLQMNSLRPEDTALYYCTIGGSLSRSSQGTLVTVSS

SEQ ID NO: 48 - protein, 115 aa, synthetic construct
EVQLVESGGGVVQPGGSLRLSCAASGFTFRSFGMSWVRQAPGAGPEWVSSISGSGSDTLYADSVKGRFTISRDNSKNTLYLQMNSLRPEDTALYYCTIGGSLSRSSQGTLVTVSS

SEQ ID NO: 49 - protein, 115 aa, synthetic construct
EVQLVESGGGVVQPGGSLRLSCAASGFTFRSFGMSWVRQAPGKGPEWVSSISGSGSDTLYADSVEGRFTISRDNSKNTLYLQMNSLRPEDTALYYCTIGGSLSRSSQGTLVTVSS

SEQ ID NO: 50 - protein, 115 aa, synthetic construct
EVQLVESGGGVVQPGGSLRLSCAASGFTFRSFGMSWVRQAPGKGPEWVSSISGSGSDTLYADSVQGRFTISRDNSKNTLYLQMNSLRPEDTALYYCTIGGSLSRSSQGTLVTVSS

SEQ ID NO: 51 - protein, 115 aa, synthetic construct
EVQLVESGGGVVQPGGSLRLSCAASGFTFRSFGMSWVRQAPGKGPEWVSSISGSGSDTLYADSVHGRFTISRDNSKNTLYLQMNSLRPEDTALYYCTIGGSLSRSSQGTLVTVSS

SEQ ID NO: 52 - protein, 115 aa, synthetic construct
EVQLVESGGGVVQPGGSLRLSCAASGFTFRSFGMSWVRQAPGKGPEWVSSISGSGSDTLYADSVKGRFTISRDNSENTLYLQMNSLRPEDTALYYCTIGGSLSRSSQGTLVTVSS

SEQ ID NO: 53 - protein, 115 aa, synthetic construct
EVQLVESGGGVVQPGGSLRLSCAASGFTFRSFGMSWVRQAPGKGPEWVSSISGSGSDTLYADSVKGRFTISRDNSQNTLYLQMNSLRPEDTALYYCTIGGSLSRSSQGTLVTVSS

SEQ ID NO: 54 - protein, 115 aa, synthetic construct
EVQLVESGGGVVQPGGSLRLSCAASGFTFRSFGMSWVRQAPGKGPEWVSSISGSGSDTLYADSVKGRFTISRDNSRNTLYLQMNSLRPEDTALYYCTIGGSLSRSSQGTLVTVSS

SEQ ID NO: 55 - protein, 115 aa, synthetic construct
EVQLVESGGGVVQPGGSLRLSCAASGFTFRSFGMSWVRQAPGKGPEWVSSISGSGSDTLYADSVKGRFTISRDNSANTLYLQMNSLRPEDTALYYCTIGGSLSRSSQGTLVTVSS

SEQ ID NO: 56 - protein, 115 aa, synthetic construct
EVQLVESGGGVVQPGGSLRLSCAASGFTFRSFGMSWVRQAPGRGPEWVSSISGSGSDTLYADSVKGRFTISRDNSKNTLYLQMNSLRPEDTALYYCTIGGSLSRSSQGTLVTVSS

SEQ ID NO: 57 - protein, 115 aa, synthetic construct
EVQLVESGGGVVQPGGSLRLSCAASGFTFRSFGMSWVRQAPGKGPEWVSSISGSGSDTLYADSVRGRFTISRDNSKNTLYLQMNSLRPEDTALYYCTIGGSLSRSSQGTLVTVSS

SEQ ID NO: 58 - protein, 115 aa, synthetic construct
EVQLVESGGGVVQPGGSLRLSCAASGFTFRSFGMSWVRQAPGKGPEWVSSISGSGSDTLYADSVAGRFTISRDNSKNTLYLQMNSLRPEDTALYYCTIGGSLSRSSQGTLVTVSS

SEQ ID NO: 59 - protein, 115 aa, synthetic construct
EVQLVESGGGVVQPGGSLRLSCAASGFTFRSFGMSWVRQAPGKGPEWVSSISGSGSDTLYADSVKGRFTISRDNSHNTLYLQMNSLRPEDTALYYCTIGGSLSRSSQGTLVTVSS

SEQ ID NO: 60 - protein, 115 aa, synthetic construct
EVQLVESGGGVVQPGGSLRLSCAASGFTFRSFGMSWVRQAPGRGPEWVSSISGSGSDTLYADSVRGRFTISRDNSKNTLYLQMNSLRPEDTALYYCTIGGSLSRSSQGTLVTVSS

SEQ ID NO: 61 - protein, 115 aa, synthetic construct
EVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 62 - protein, 115 aa, synthetic construct
EVQLVESGGGVVQPGGSLRLSCAASGFTFRSFGMSWVRQAPGRGPEWVSSISGSGSDTLYADSVKGRFTISRDNSRNTLYLQMNSLRPEDTALYYCTIGGSLSRSSQGTLVTVSS

SEQ ID NO: 63 - protein, 115 aa, synthetic construct
EVQLVESGGGVVQPGGSLRLSCAASGFTFRSFGMSWVRQAPGRGPEWVSSISGSGSDTLYADSVRGRFTISRDKSRNTLYLQMNSLRPEDTALYYCTIGGSLSRSSQGTLVTVSS

SEQ ID NO: 64 - protein, 115 aa, synthetic construct
EVQLVESGGGVVQPGGSLRLSCAASGFTFRSFGMSWVRQAPGKGPEWVSSISGSGSDTLYADSVRGRFTISRDNSRNTLYLQMNSLRPEDTALYYCTIGGSLSRSSQGTLVTVSS

SEQ ID NO: 65 - protein, 115 aa, synthetic construct
EVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSKGTLVTVSS

SEQ ID NO: 66 - protein, 115 aa, synthetic construct
EVQLVESGGGVVQPGGSLRLSCAASGFTFRSFGMSWVRQAPGRGKEWVSSISGSGSDTLYADSVRGRFTISRDNSRNTLYLQMNSLRPEDTALYYCTIGGSLSRSSQGTLVTVSS

SEQ ID NO: 67 - protein, 116 aa, synthetic construct
EVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSSK

SEQ ID NO: 68 - protein, 122 aa, synthetic construct
DVQLVESGGGVVQPGGSLRLSCTASGFTFSTADMGWFRQAPGKGREFVARISGIDGTTYYDEPVKGRFTISRDNSKNTVYLQMNSLRPEDTALYYCRSPRYADQWSAYDYWGQGTLVTVSSA

SEQ ID NO: 69 - protein, 121 aa, synthetic construct
DVQLVESGGGVVQPGGSLRLSCTASGFTFSTADMGWFRQAPGKGREFVARISGIDGTTYYDEPVKGRFTISRDKSKNTVYLQMNSLKPEDTALYYCRSPRYADQWSAYDYWGQGTLVTVSS

SEQ ID NO: 70 - protein, 121 aa, synthetic construct
DVQLVESGGGVVQPGGSLKLSCTASGFTFSTADMGWFRQAPGKGREFVARISGIDGTTYYDEPVKGRFTISRDKSKNTVYLQMNSLKPEDTALYYCRSPRYADQWSAYDYWGQGTLVTVSS

SEQ ID NO: 71 - protein, 121 aa, synthetic construct
DVQLVESGGGVVQPGGSLKLSCTASGFTFSTADMGWFRQAPGKGREFVARISGIDGTTYYDEPVKGRFTISRDNSKNTVYLQMNSLKPEDTALYYCRSPRYADQWSAYDYWGKGTLVTVSS

SEQ ID NO: 72 - protein, 121 aa, synthetic construct
DVQLVESGGGVVQPGGSLKLSCTASGFTFSTADMGWFRQAPGKGREFVARISGIDGTTYYDEPVKGRFTISKDNSKNTVYLQMNSLKPEDTALYYCRSPRYADQWSAYDYWGKGTLVTVSS

SEQ ID NO: 73 - protein, 121 aa, synthetic construct
DVQLVESGGGVVQPGGSLKLSCTASGFTFSTADMGWFRQAPGKGREFVARISGIDGTTYYDEPVKGRFTISRDKSKNTVYLQMNSLKPEDTALYYCRSPRYADQWSAYDYWGKGTLVTVSS

SEQ ID NO: 74 - protein, 121 aa, synthetic construct
DVQLVESGGGVVQPGGSLKLSCTASGFTFSTADMGWFRQAPGKGREFVARISGIDGTTYYDEPVKGRFTISKDKSKNTVYLQMNSLKPEDTALYYCRSPRYADQWSAYDYWGKGTLVTVSS

SEQ ID NO: 75 - protein, 121 aa, synthetic construct
DVQLVESGGGVVQPGGSLKLSCTASGFTFSTADMGWFRQAPGKGKEFVARISGIDGTTYYDEPVKGRFTISRDKSKNTVYLQMNSLKPEDTALYYCRSPRYADQWSAYDYWGKGTLVTVSS

SEQ ID NO: 76-77 - skipped (2 entries)

SEQ ID NO: 78 - protein, 121 aa, synthetic construct
DVQLVESGGGVVQPGGSLKLSCTASGFTFSTADMGWFRQAPGKGKEFVARISGIDGTTYYDEPVKGRFTISKDKSKNTVYLQMNSLKPEDTALYYCRSPRYADQWSAYDYWGKGTLVTVSS

SEQ ID NO: 79 - protein, 10 aa, synthetic construct
GLTFSTYTMG

SEQ ID NO: 80 - protein, 10 aa, synthetic construct
AIIWSGSNTY

SEQ ID NO: 81 - protein, 15 aa, synthetic construct
QHFGPIGLTTRGYHY

SEQ ID NO: 82 - protein, 10 aa, synthetic construct
GHTFSEYALG

SEQ ID NO: 83 - protein, 10 aa, synthetic construct
AINWGGGWTY

SEQ ID NO: 84 - protein, 14 aa, synthetic construct
SSDYAGGNPTGYPY

SEQ ID NO: 85 - protein, 10 aa, synthetic construct
GFTFRSFGMS

SEQ ID NO: 86 - protein, 10 aa, synthetic construct
SISGSGSDTL

SEQ ID NO: 87 - protein, 6 aa, synthetic construct
GGSLSR

SEQ ID NO: 88 - protein, 10 aa, synthetic construct
GFTFSTADMG

SEQ ID NO: 89 - protein, 10 aa, synthetic construct
RISGIDGTTY

SEQ ID NO: 90 - protein, 12 aa, synthetic construct
PRYADQWSAYDY

SEQ ID NO: 91-107 - skipped (17 entries)

SEQ ID NO: 108 - protein, 5 aa, synthetic construct
  REGION 1..5: note = 5GS linker
GGGGS

SEQ ID NO: 109 - protein, 7 aa, synthetic construct
  REGION 1..7: note = 7GS linker
SGGSGGS

SEQ ID NO: 110 - protein, 8 aa, synthetic construct
  REGION 1..8: note = 8GS linker
GGGGSGGS

SEQ ID NO: 111 - protein, 9 aa, synthetic construct
  REGION 1..9: note = 9GS linker
GGGGSGGGS

SEQ ID NO: 112 - protein, 10 aa, synthetic construct
  REGION 1..10: note = 10GS linker
GGGGSGGGGS

SEQ ID NO: 113 - protein, 15 aa, synthetic construct
  REGION 1..15: note = 15GS linker
GGGGSGGGGSGGGGS

SEQ ID NO: 114 - protein, 18 aa, synthetic construct
  REGION 1..18: note = 18GS linker
GGGGSGGGGSGGGGSGGS

SEQ ID NO: 115 - protein, 20 aa, synthetic construct
  REGION 1..20: note = 20GS linker
GGGGSGGGGSGGGGSGGGGS

SEQ ID NO: 116 - protein, 25 aa, synthetic construct
  REGION 1..25: note = 25GS linker
GGGGSGGGGSGGGGSGGGGSGGGGS

SEQ ID NO: 117 - protein, 30 aa, synthetic construct
  REGION 1..30: note = 30GS linker
GGGGSGGGGSGGGGSGGGGSGGGGSGGGGS

SEQ ID NO: 118 - protein, 35 aa, synthetic construct
  REGION 1..35: note = 35GS linker
GGGGSGGGGSGGGGSGGGGSGGGGSGGGGSGGGGS

SEQ ID NO: 119 - protein, 40 aa, synthetic construct
  REGION 1..40: note = 40GS linker
GGGGSGGGGSGGGGSGGGGSGGGGSGGGGSGGGGSGGGGS

SEQ ID NO: 120 - protein, 15 aa, synthetic construct
EPKSCDKTHTCPPCP

SEQ ID NO: 121 - protein, 24 aa, synthetic construct
GGGGSGGGSEPKSCDKTHTCPPCP

SEQ ID NO: 122 - protein, 12 aa, synthetic construct
EPKTPKPQPAAA

SEQ ID NO: 123 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 124 - protein, 4 aa, synthetic construct
  REGION 1..4: note = Motif from Table 2
KERE

SEQ ID NO: 125 - protein, 4 aa, synthetic construct
  REGION 1..4: note = Motif from Table 2
KQRE

SEQ ID NO: 126 - protein, 4 aa, synthetic construct
  REGION 1..4: note = Motif from Table 2
GLEW

SEQ ID NO: 127 - protein, 5 aa, synthetic construct
  REGION 1..5: note = Motif from Table 2
KEREL

SEQ ID NO: 128 - protein, 5 aa, synthetic construct
  REGION 1..5: note = Motif from Table 2
KEREF

SEQ ID NO: 129 - protein, 5 aa, synthetic construct
  REGION 1..5: note = Motif from Table 2
KQREL

SEQ ID NO: 130 - protein, 5 aa, synthetic construct
  REGION 1..5: note = Motif from Table 2
KQREF

SEQ ID NO: 131 - protein, 5 aa, synthetic construct
  REGION 1..5: note = Motif from Table 2
KEREG

SEQ ID NO: 132 - protein, 5 aa, synthetic construct
  REGION 1..5: note = Motif from Table 2
KQREW

SEQ ID NO: 133 - protein, 5 aa, synthetic construct
  REGION 1..5: note = Motif from Table 2
KQREG

SEQ ID NO: 134 - protein, 4 aa, synthetic construct
  REGION 1..4: note = Motif from Table A-0
TERE

SEQ ID NO: 135 - protein, 5 aa, synthetic construct
  REGION 1..5: note = Motif from Table 2
TEREL

SEQ ID NO: 136 - protein, 4 aa, synthetic construct
  REGION 1..4: note = Motif from Table 2
TQRE

SEQ ID NO: 137 - protein, 5 aa, synthetic construct
  REGION 1..5: note = Motif from Table 2
TQREL

SEQ ID NO: 138 - protein, 4 aa, synthetic construct
  REGION 1..4: note = Motif from Table 2
KECE

SEQ ID NO: 139 - protein, 5 aa, synthetic construct
  REGION 1..5: note = Motif from Table 2
KECEL

SEQ ID NO: 140 - protein, 5 aa, synthetic construct
  REGION 1..5: note = Motif from Table 2
KECER

SEQ ID NO: 141 - protein, 4 aa, synthetic construct
  REGION 1..4: note = Motif from Table 2
KQCE

SEQ ID NO: 142 - protein, 5 aa, synthetic construct
  REGION 1..5: note = Motif from Table 2
KQCEL

SEQ ID NO: 143 - protein, 4 aa, synthetic construct
  REGION 1..4: note = Motif from Table 2
RERE

SEQ ID NO: 144 - protein, 5 aa, synthetic construct
  REGION 1..5: note = Motif from Table 2
REREG

SEQ ID NO: 145 - protein, 4 aa, synthetic construct
  REGION 1..4: note = Motif from Table 2
RQRE

SEQ ID NO: 146 - protein, 5 aa, synthetic construct
  REGION 1..5: note = Motif from Table 2
RQREL

SEQ ID NO: 147 - protein, 5 aa, synthetic construct
  REGION 1..5: note = Motif from Table 2
RQREF

SEQ ID NO: 148 - protein, 5 aa, synthetic construct
  REGION 1..5: note = Motif from Table 2
RQREW

SEQ ID NO: 149 - protein, 4 aa, synthetic construct
  REGION 1..4: note = Motif from Table 2
QERE

SEQ ID NO: 150 - protein, 5 aa, synthetic construct
  REGION 1..5: note = Motif from Table 2
QEREG

SEQ ID NO: 151 - protein, 4 aa, synthetic construct
  REGION 1..4: note = Motif from Table 2
QQRE

SEQ ID NO: 152 - protein, 5 aa, synthetic construct
  REGION 1..5: note = Motif from Table 2
QQREW

SEQ ID NO: 153 - protein, 5 aa, synthetic construct
  REGION 1..5: note = Motif from Table 2
QQREL

SEQ ID NO: 154 - protein, 5 aa, synthetic construct
  REGION 1..5: note = Motif from Table 2
QQREF

SEQ ID NO: 155 - protein, 4 aa, synthetic construct
  REGION 1..4: note = Motif from Table 2
KGRE

SEQ ID NO: 156 - protein, 5 aa, synthetic construct
  REGION 1..5: note = Motif from Table 2
KGREG

SEQ ID NO: 157 - protein, 4 aa, synthetic construct
  REGION 1..4: note = Motif from Table 2
KDRE

SEQ ID NO: 158 - protein, 5 aa, synthetic construct
  REGION 1..5: note = Motif from Table 2
KDREV

SEQ ID NO: 159 - protein, 5 aa, synthetic construct
  REGION 1..5: note = Motif from Table 2
DECKL

SEQ ID NO: 160 - protein, 5 aa, synthetic construct
  REGION 1..5: note = Motif from Table 2
NVCEL

SEQ ID NO: 161 - protein, 4 aa, synthetic construct
  REGION 1..4: note = Motif from Table 2
GVEW

SEQ ID NO: 162 - protein, 4 aa, synthetic construct
  REGION 1..4: note = Motif from Table 2
EPEW

SEQ ID NO: 163 - protein, 4 aa, synthetic construct
  REGION 1..4: note = Motif from Table 2
GLER

SEQ ID NO: 164 - protein, 4 aa, synthetic construct
  REGION 1..4: note = Motif from Table 2
DQEW

SEQ ID NO: 165 - protein, 4 aa, synthetic construct
  REGION 1..4: note = Motif from Table 2
DLEW

SEQ ID NO: 166 - protein, 4 aa, synthetic construct
  REGION 1..4: note = Motif from Table 2
GIEW

SEQ ID NO: 167 - protein, 4 aa, synthetic construct
  REGION 1..4: note = Motif from Table 2
ELEW

SEQ ID NO: 168 - protein, 4 aa, synthetic construct
  REGION 1..4: note = Motif from Table 2
GPEW

SEQ ID NO: 169 - protein, 4 aa, synthetic construct
  REGION 1..4: note = Motif from Table 2
EWLP

SEQ ID NO: 170 - protein, 4 aa, synthetic construct
  REGION 1..4: note = Motif from Table 2
GPER

SEQ ID NO: 171 - protein, 4 aa, synthetic construct
  REGION 1..4: note = Motif from Table 2
GLER

SEQ ID NO: 172 - protein, 4 aa, synthetic construct
  REGION 1..4: note = Motif from Table 2
ELEW

SEQ ID NO: 173 - protein, 124 aa, synthetic construct
QVQLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 174 - protein, 125 aa, synthetic construct
GQVQLEESGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 175 - protein, 124 aa, synthetic construct
QVQLEESGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 176 - protein, 125 aa, synthetic construct
QVQLEESGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSSK

SEQ ID NO: 177 - protein, 124 aa, synthetic construct
QVQLEESGGGSVQTGGSLKLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 178 - protein, 124 aa, synthetic construct
QVQLEESGGKSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 179 - protein, 124 aa, synthetic construct
QVQLEESGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGKGTQVTVSS

SEQ ID NO: 180 - protein, 124 aa, synthetic construct
QVQLEESGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTKVTVSS

SEQ ID NO: 181 - protein, 124 aa, synthetic construct
QVQLEESGGGSKQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 182 - protein, 124 aa, synthetic construct
QVQLEESGGGSVKTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 183 - protein, 124 aa, synthetic construct
QVQLEESGGGSVQTGGSLRLTCAASGRTSRSYGMGWFRQAPGKEREFVSGISWRGDSTGYADSVRGRFTISRDNARNTVDLQMNSLRPEDTAIYYCAAAAGSAWYGTLYEYDYWGQGTQVTVSS

SEQ ID NO: 184 - protein, 124 aa, synthetic construct
QVQLEESGGGSVQTGGSLRLTCAASGRTSRSYGMGWFRQAPGRKREFVSGISWRGDSTGYADSVRGRFTISRDNARNTVDLQMNSLRPEDTAIYYCAAAAGSAWYGTLYEYDYWGQGTQVTVSS

SEQ ID NO: 185 - protein, 124 aa, synthetic construct
QVQLEESGGGSVQTGGSLRLTCAASGRTSRSYGMGWFRQAPGREKEFVSGISWRGDSTGYADSVRGRFTISRDNARNTVDLQMNSLRPEDTAIYYCAAAAGSAWYGTLYEYDYWGQGTQVTVSS

SEQ ID NO: 186 - protein, 124 aa, synthetic construct
QVQLKESGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 187 - protein, 124 aa, synthetic construct
QVQLEESGGGSVQTGGSLRLTCAASGRTSRSYGMGWFRQAPGREREFVSGISWRGDSTGYAKSVRGRFTISRDNARNTVDLQMNSLRPEDTAIYYCAAAAGSAWYGTLYEYDYWGQGTQVTVSS

SEQ ID NO: 188 - protein, 124 aa, synthetic construct
QVQLEESGGGSVQTGGSLRLTCAASGRTSRSYGMGWFRQAPGREREFVSGISWRGDSTGYADSVKGRFTISRDNARNTVDLQMNSLRPEDTAIYYCAAAAGSAWYGTLYEYDYWGQGTQVTVSS

SEQ ID NO: 189 - protein, 124 aa, synthetic construct
QVQLEEKGGGSVQTGGSLRLTCAASGRTSRSYGMGWFRQAPGREREFVSGISWRGDSTGYADSVRGRFTISRDNARNTVDLQMNSLRPEDTAIYYCAAAAGSAWYGTLYEYDYWGQGTQVTVSS

SEQ ID NO: 190 - protein, 124 aa, synthetic construct
QVQLEESGGGSVQTGGSLRLTCAASGRTSRSYGMGWFRQAPGREREFVSGISWRGDSTGYADSVRGRFTIKRDNARNTVDLQMNSLRPEDTAIYYCAAAAGSAWYGTLYEYDYWGQGTQVTVSS

SEQ ID NO: 191 - protein, 124 aa, synthetic construct
QVQLEESGGGSVQTGGSLRLTCAASGRTSRSYGMGWFRQAPGREREFVSGISWRGDSTGYADSVRGRFTISKDNARNTVDLQMNSLRPEDTAIYYCAAAAGSAWYGTLYEYDYWGQGTQVTVSS

SEQ ID NO: 192 - protein, 124 aa, synthetic construct
QVQLEESGGGSVQTGGSLRLTCAASGRTSRSYGMGWFRQAPGREREFVSGISWRGDSTGYADSVRGRFTISRDKARNTVDLQMNSLRPEDTAIYYCAAAAGSAWYGTLYEYDYWGQGTQVTVSS

SEQ ID NO: 193 - protein, 124 aa, synthetic construct
QVQLEESGGGSVQTGGSLRLTCAASGRTSRSYGMGWFRQAPGREREFVSGISWRGDSTGYADSVRGRFTISRDNAKNTVDLQMNSLRPEDTAIYYCAAAAGSAWYGTLYEYDYWGQGTQVTVSS

SEQ ID NO: 194 - protein, 124 aa, synthetic construct
QVQLEESGGGSVQTGGSLRLTCAASGRTSRSYGMGWFRQAPGREREFVSGISWRGDSTGYADSVRGRFTISRDNARNTVDLQMNSLKPEDTAIYYCAAAAGSAWYGTLYEYDYWGQGTQVTVSS

SEQ ID NO: 195 - protein, 117 aa, synthetic construct
EVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSS

SEQ ID NO: 196 - protein, 118 aa, synthetic construct
QVQLVESGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSSK

SEQ ID NO: 197 - protein, 117 aa, synthetic construct
QVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSS

SEQ ID NO: 198 - protein, 118 aa, synthetic construct
GQVQLVESGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 199 - protein, 117 aa, synthetic construct
QVQLVESGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 200 - protein, 117 aa, synthetic construct
QVQLKESGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 201 - protein, 117 aa, synthetic construct
QVQLVEKGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 202 - protein, 117 aa, synthetic construct
QVQLVESGGKLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 203 - protein, 117 aa, synthetic construct
QVQLVESGGGLKQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 204 - protein, 117 aa, synthetic construct
QVQLVESGGGLVKPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 205 - protein, 117 aa, synthetic construct
QVQLVESGGGLVQPGGSLKLSCAASGLTFSTNPMYWYRQAPGRQRELVASISSRGITNYADSVRGRFTISRDNSRNTVYLQMNSLRPEDTAVYYCRLASLSSGTVYWGQGTLVTVSS

SEQ ID NO: 206 - protein, 117 aa, synthetic construct
QVQLVESGGGLVQPGGSLRLSCAASGLTFSTNPMYWYRQAPGKQRELVASISSRGITNYADSVRGRFTISRDNSRNTVYLQMNSLRPEDTAVYYCRLASLSSGTVYWGQGTLVTVSS

SEQ ID NO: 207 - protein, 117 aa, synthetic construct
QVQLVESGGGLVQPGGSLRLSCAASGLTFSTNPMYWYRQAPGRKRELVASISSRGITNYADSVRGRFTISRDNSRNTVYLQMNSLRPEDTAVYYCRLASLSSGTVYWGQGTLVTVSS

SEQ ID NO: 208 - protein, 117 aa, synthetic construct
QVQLVESGGGLVQPGGSLRLSCAASGLTFSTNPMYWYRQAPGRQKELVASISSRGITNYADSVRGRFTISRDNSRNTVYLQMNSLRPEDTAVYYCRLASLSSGTVYWGQGTLVTVSS

SEQ ID NO: 209 - protein, 117 aa, synthetic construct
QVQLVESGGGLVQPGGSLRLSCAASGLTFSTNPMYWYRQAPGRQRELVASISSRGITNYAKSVRGRFTISRDNSRNTVYLQMNSLRPEDTAVYYCRLASLSSGTVYWGQGTLVTVSS

SEQ ID NO: 210 - protein, 117 aa, synthetic construct
QVQLVESGGGLVQPGGSLRLSCAASGLTFSTNPMYWYRQAPGRQRELVASISSRGITNYADSVKGRFTISRDNSRNTVYLQMNSLRPEDTAVYYCRLASLSSGTVYWGQGTLVTVSS

SEQ ID NO: 211 - protein, 117 aa, synthetic construct
QVQLVESGGGLVQPGGSLRLSCAASGLTFSTNPMYWYRQAPGRQRELVASISSRGITNYADSVRGRFTIKRDNSRNTVYLQMNSLRPEDTAVYYCRLASLSSGTVYWGQGTLVTVSS

SEQ ID NO: 212 - protein, 117 aa, synthetic construct
QVQLVESGGGLVQPGGSLRLSCAASGLTFSTNPMYWYRQAPGRQRELVASISSRGITNYADSVRGRFTISKDNSRNTVYLQMNSLRPEDTAVYYCRLASLSSGTVYWGQGTLVTVSS

SEQ ID NO: 213 - protein, 117 aa, synthetic construct
QVQLVESGGGLVQPGGSLRLSCAASGLTFSTNPMYWYRQAPGRQRELVASISSRGITNYADSVRGRFTISRDKSRNTVYLQMNSLRPEDTAVYYCRLASLSSGTVYWGQGTLVTVSS

SEQ ID NO: 214 - protein, 117 aa, synthetic construct
QVQLVESGGGLVQPGGSLRLSCAASGLTFSTNPMYWYRQAPGRQRELVASISSRGITNYADSVRGRFTISRDNSKNTVYLQMNSLRPEDTAVYYCRLASLSSGTVYWGQGTLVTVSS

SEQ ID NO: 215 - protein, 117 aa, synthetic construct
QVQLVESGGGLVQPGGSLRLSCAASGLTFSTNPMYWYRQAPGRQRELVASISSRGITNYADSVRGRFTISRDNSRNTVYLQMNSLKPEDTAVYYCRLASLSSGTVYWGQGTLVTVSS

SEQ ID NO: 216 - protein, 117 aa, synthetic construct
QVQLVESGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGKGTLVTVSS

SEQ ID NO: 217 - protein, 117 aa, synthetic construct
QVQLVESGGGLVQPGGSLRLSCAASGLTFSTNPMYWYRQAPGRQRELVASISSRGITNYADSVRGRFTISRDNSRNTVYLQMNSLRPEDTAVYYCRLASLSSGTVYWGQGTKVTVSS

SEQ ID NO: 218 - protein, 117 aa, synthetic construct
QVQLVESGGGLVQPGGSLRLSCAASGLTFSTNPMYWYRQAPGRQKELVASISSRGITNYADSVRGRFTISRDKSRNTVYLQMNSLRPEDTAVYYCRLASLSSGTVYWGQGTLVTVSS

SEQ ID NO: 219 - protein, 127 aa, synthetic construct
EVQLVESGGGVVQPGGSLRLSCAASGRTFSSYAMGWFRQAPGKEREFVAAISWSDGSTYYADSVKGRFTISRDNAKNTVYLQMNSLRPEDTALYYCAADLTSTNPGSYIYIWAYDYWGQGTLVTVSS

SEQ ID NO: 220 - protein, 127 aa, synthetic construct
QVQLVESGGGVVQPGGSLRLSCAASGRTFSSYAMGWFRQAPGREKEFVAAISWSDGSTYYADSVRGRFTISRDNARNTVYLQMNSLRPEDTALYYCAADLTSTNPGSYIYIWAYDYWGQGTKVTVSS

SEQ ID NO: 221 - protein, 127 aa, synthetic construct
QVQLVESGGGVVQPGGSLRLSCAASGRTFSSYAMGWFRQAPGREKEFVAAISWSDGSTYYADSVRGRFTISRDKARNTVYLQMNSLRPEDTALYYCAADLTSTNPGSYIYIWAYDYWGQGTLVTVSS

SEQ ID NO: 222 - protein, 127 aa, synthetic construct
QVQLVESGGGVVKPGGSLRLSCAASGRTFSSYAMGWFRQAPGREKEFVAAISWSDGSTYYADSVRGRFTISRDNARNTVYLQMNSLRPEDTALYYCAADLTSTNPGSYIYIWAYDYWGQGTLVTVSS

SEQ ID NO: 223 - protein, 127 aa, synthetic construct
QVQLVESGGGVVQPGGSLRLSCAASGRTFSSYAMGWFRQAPGREKEFVAAISWSDGSTYYADSVRGRFTISRDNARNTVYLQMNSLRPEDTALYYCAADLTSTNPGSYIYIWAYDYWGQGTLVTVSS

SEQ ID NO: 224 - protein, 132 aa, synthetic construct
EVQLVESGGGVVQPGGSLRLSCAASGFTFEDYAIGWFRQAPGREREEVSCIRTYDEQTYYADSVRGRFTISRDNARNTVSLQMNSLRPEDTALYYCAAGSYYACAYYSRPDPSEGHVDLDYWGQGTLVTVSS

SEQ ID NO: 225 - protein, 117 aa, synthetic construct; identical to SEQ ID NO: 195

SEQ ID NO: 226 - protein, 124 aa, synthetic construct; identical to SEQ ID NO: 173

SEQ ID NO: 227 - protein, 127 aa, synthetic construct; identical to SEQ ID NO: 219

SEQ ID NO: 228 - protein, 115 aa, synthetic construct
QVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 229 - protein, 115 aa, synthetic construct
QVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 230 - protein, 115 aa, synthetic construct
QVQLVESGGKVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 231 - protein, 115 aa, synthetic construct
QVQLVESGGGVVKPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 232 - protein, 115 aa, synthetic construct
QVQLVESGGGVVQPGGSLRLSCAASGFTFRSFGMSWVRQAPGKGPEWVSSISGSGSDTLYADSVRGRFTISRDNSRNTLYLQMNSLRPEDTALYYCTIGGSLSRSSQGTLVTVSS

SEQ ID NO: 233 - protein, 115 aa, synthetic construct
QVQLVESGGGVVQPGGSLRLSCAASGFTFRSFGMSWVRQAPGRGPEWVSSISGSGSDTLYADSVRGRFTISRDNSKNTLYLQMNSLRPEDTALYYCTIGGSLSRSSQGTLVTVSS

SEQ ID NO: 234 - protein, 115 aa, synthetic construct
QVQLKESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 235 - protein, 115 aa, synthetic construct
QVQLVEKGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 236 - protein, 115 aa, synthetic construct
QVQLVESGGGVKQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 237 - protein, 115 aa, synthetic construct
QVQLVESGGGVVQPGGSLKLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 238 - protein, 115 aa, synthetic construct
QVQLVESGGGVVQPGGSLRLSCAASGFTFRSFGMSWVRQAPGRKPEWVSSISGSGSDTLYADSVRGRFTISRDNSRNTLYLQMNSLRPEDTALYYCTIGGSLSRSSQGTLVTVSS

SEQ ID NO: 239 - protein, 115 aa, synthetic construct
QVQLVESGGGVVQPGGSLRLSCAASGFTFRSFGMSWVRQAPGRGKEWVSSISGSGSDTLYADSVRGRFTISRDNSRNTLYLQMNSLRPEDTALYYCTIGGSLSRSSQGTLVTVSS

SEQ ID NO: 240 - protein, 115 aa, synthetic construct
QVQLVESGGGVVQPGGSLRLSCAASGFTFRSFGMSWVRQAPGRGPEWVSSISGSGSDTLYAKSVRGRFTISRDNSRNTLYLQMNSLRPEDTALYYCTIGGSLSRSSQGTLVTVSS

SEQ ID NO: 241 - protein, 115 aa, synthetic construct
QVQLVESGGGVVQPGGSLRLSCAASGFTFRSFGMSWVRQAPGRGPEWVSSISGSGSDTLYADSVKGRFTISRDNSRNTLYLQMNSLRPEDTALYYCTIGGSLSRSSQGTLVTVSS

SEQ ID NO: 242 - protein, 115 aa, synthetic construct
QVQLVESGGGVVQPGGSLRLSCAASGFTFRSFGMSWVRQAPGRGPEWVSSISGSGSDTLYADSVRGRFTIKRDNSRNTLYLQMNSLRPEDTALYYCTIGGSLSRSSQGTLVTVSS

SEQ ID NO: 243 - protein, 115 aa, synthetic construct
QVQLVESGGGVVQPGGSLRLSCAASGFTFRSFGMSWVRQAPGRGPEWVSSISGSGSDTLYADSVRGRFTISKDNSRNTLYLQMNSLRPEDTALYYCTIGGSLSRSSQGTLVTVSS

SEQ ID NO: 244 - protein, 115 aa, synthetic construct
QVQLVESGGGVVQPGGSLRLSCAASGFTFRSFGMSWVRQAPGRGPEWVSSISGSGSDTLYADSVRGRFTISRDKSRNTLYLQMNSLRPEDTALYYCTIGGSLSRSSQGTLVTVSS

SEQ ID NO: 245 - protein, 115 aa, synthetic construct
QVQLVESGGGVVQPGGSLRLSCAASGFTFRSFGMSWVRQAPGRGPEWVSSISGSGSDTLYADSVRGRFTISRDNSRNTLYLQMNSLKPEDTALYYCTIGGSLSRSSQGTLVTVSS

SEQ ID NO: 246 - protein, 115 aa, synthetic construct
QVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSKGTLVTVSS

SEQ ID NO: 247 - protein, 115 aa, synthetic construct
QVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTKVTVSS

SEQ ID NO: 248 - protein, 116 aa, synthetic construct
QVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSSK

SEQ ID NO: 249 - protein, 116 aa, synthetic construct
GQVQLVESGGGVVQPGGSLRLSCAASGFTFRSFGMSWVRQAPGRGPEWVSSISGSGSDTLYADSVRGRFTISRDNSRNTLYLQMNSLRPEDTALYYCTIGGSLSRSSQGTLVTVSS

SEQ ID NO: 250 - protein, 132 aa, synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAYYSRPDPSEGHVDLDYWGQGTLVTVSS

SEQ ID NO: 251 - protein, 10 aa, synthetic construct
GRTSRSYGMG

SEQ ID NO: 252 - protein, 10 aa, synthetic construct
GISWRGDSTG

SEQ ID NO: 253 - protein, 15 aa, synthetic construct
AAGSAWYGTLYEYDY

SEQ ID NO: 254 - protein, 10 aa, synthetic construct
GLTFSTNPMY

SEQ ID NO: 255 - protein, 9 aa, synthetic construct
SISSRGITN

SEQ ID NO: 256 - protein, 9 aa, synthetic construct
ASLSSGTVY

SEQ ID NO: 257 - protein, 10 aa, synthetic construct
GRTFSSYAMG

SEQ ID NO: 258 - protein, 10 aa, synthetic construct
AISWSDGSTY

SEQ ID NO: 259 - protein, 18 aa, synthetic construct
DLTSTNPGSYIYIWAYDY

SEQ ID NO: 260 - protein, 10 aa, synthetic construct
GFTFEDYAIG

SEQ ID NO: 261 - protein, 10 aa, synthetic construct
GFTFEDYAIG

SEQ ID NO: 262 - protein, 10 aa, synthetic construct
CIRTYDEQTY

SEQ ID NO: 263 - protein, 23 aa, synthetic construct
GSYYACAYYSRPDPSEGHVDLDY

SEQ ID NO: 264 - protein, 5 aa, synthetic construct
TYTMG

SEQ ID NO: 265 - protein, 17 aa, synthetic construct
AIIWSGSNTYYADSVHG

SEQ ID NO: 266 - protein, 5 aa, synthetic construct
EYALG

SEQ ID NO: 267 - protein, 17 aa, synthetic construct
AINWGGGWTYYADSVKG

SEQ ID NO: 268 - protein, 5 aa, synthetic construct
SFGMS

SEQ ID NO: 269 - protein, 17 aa, synthetic construct
SISGSGSDTLYADSVKG

SEQ ID NO: 270 - protein, 5 aa, synthetic construct
TADMG

SEQ ID NO: 271 - protein, 17 aa, synthetic construct
RISGIDGTTYYDEPVKG

SEQ ID NO: 272 - protein, 5 aa, synthetic construct
SYGMG

SEQ ID NO: 273 - protein, 17 aa, synthetic construct
GISWRGDSTGYADSVRG

SEQ ID NO: 274 - protein, 5 aa, synthetic construct
TNPMY

SEQ ID NO: 275 - protein, 16 aa, synthetic construct
SISSRGITNYADSVRG

SEQ ID NO: 276 - protein, 5 aa, synthetic construct
SYAMG

SEQ ID NO: 277 - protein, 17 aa, synthetic construct
AISWSDGSTYYADSVRG

SEQ ID NO: 278 - protein, 5 aa, Saaristattus tropicus
DYAIG

SEQ ID NO: 279 - protein, 17 aa, synthetic construct
CIRTYDEQTYYADSVKG

SEQ ID NO: 280 - protein, 17 aa, synthetic construct
AIIWSGSNTYYADSVEG

SEQ ID NO: 281 - protein, 17 aa, synthetic construct
AIIWSGSNTYYADSVRG

SEQ ID NO: 282 - protein, 17 aa, synthetic construct
AIIWSGSNTYYADSVQG

SEQ ID NO: 283 - protein, 17 aa, synthetic construct
AIIWSGSNTYYADSVAG

SEQ ID NO: 284 - protein, 17 aa, synthetic construct
AINWGGGWTYYADSVEG

SEQ ID NO: 285 - protein, 17 aa, synthetic construct
AINWGGGWTYYADSVQG

SEQ ID NO: 286 - protein, 17 aa, synthetic construct
AINWGGGWTYYADSVRG

SEQ ID NO: 287 - protein, 17 aa, synthetic construct
AINWGGGWTYYADSVHG

SEQ ID NO: 288 - protein, 17 aa, synthetic construct
AINWGGGWTYYADSVAG

SEQ ID NO: 289 - protein, 17 aa, synthetic construct
SISGSGSDTLYADSVEG

SEQ ID NO: 290 - protein, 17 aa, synthetic construct
SISGSGSDTLYADSVQG

SEQ ID NO: 291 - protein, 17 aa, synthetic construct
SISGSGSDTLYADSVHG

SEQ ID NO: 292 - protein, 17 aa, synthetic construct
SISGSGSDTLYADSVRG

SEQ ID NO: 293 - protein, 17 aa, synthetic construct
SISGSGSDTLYADSVAG

SEQ ID NO: 294 - protein, 17 aa, synthetic construct
SISGSGSDTLYADSVKG

SEQ ID NO: 295 - protein, 17 aa, synthetic construct
SISGSGSDTLYAKSVRG

SEQ ID NO: 296 - protein, 16 aa, synthetic construct
SISSRGITNYADSVKG

SEQ ID NO: 297 - protein, 16 aa, synthetic construct
SISSRGITNYAKSVRG

SEQ ID NO: 298 - protein, 5 aa, synthetic construct
SYAMG

SEQ ID NO: 299 - protein, 17 aa, synthetic construct
AISWSDGSTYYADSVRG

SEQ ID NO: 300 - protein, 17 aa, synthetic construct
AISWSDGSTYYADSVKG

SEQ ID NO: 301 - protein, 5 aa, synthetic construct
DYAIG

SEQ ID NO: 302 - protein, 17 aa, synthetic construct
CIRTYDEQTYYADSVKG

SEQ ID NO: 303 - protein, 17 aa, synthetic construct
CIRTYDEQTYYADSVRG

SEQ ID NO: 304 - protein, 17 aa, synthetic construct
AIIWSGSNTYYADSVKG

SEQ ID NO: 305 - protein, 17 aa, synthetic construct
GISWRGDSTGYADSVKG

SEQ ID NO: 306 - protein, 17 aa, synthetic construct
GISWRGDSTGYAKSVRG